(19) United States
(12) Patent Application Publication
(10) Pub. No.: US 2005/0209674 A1
(43) Pub. Date: Sep. 22, 2005

(54) Balloon assembly (V)

(76) Inventors: Tuvia D. Kutscher, Shoham (IL); Doron Marco, Tel-Aviv (IL)

Correspondence Address: MORRISON & FOERSTER LLP, 755 PAGE MILL RD, PALO ALTO, CA, 94304-1018, US

(21) Appl. No.: 10/919,958

(22) Filed: Aug. 16, 2004

Related U.S. Application Data
(63) Continuation-in-part of application No. 10/741,927, filed on Dec. 19, 2003.
(60) Provisional application No. 60/500,248, filed on Sep. 5, 2003.
(60) Provisional application No. 60/510,442, filed on Oct. 14, 2003.
(60) Provisional application No. 60/512,864, filed on Oct. 22, 2003.
(60) Provisional application No. 60/518,632, filed on Nov. 12, 2003.
(60) Provisional application No. 60/524,026, filed on Nov. 24, 2003.
(60) Provisional application No. 60/548,397, filed on Feb. 27, 2004.
(60) Provisional application No. 60/559,106, filed on Apr. 3, 2004.
(60) Provisional application No. 60/575,718, filed on May 27, 2004.
(60) Provisional application No. 60/500,248, filed on Sep. 5, 2003.
(60) Provisional application No. 60/512,864, filed on Oct. 22, 2003.

(51) Int. Cl. A61F 2/06

(52) U.S. Cl. 623/1.11

(57) ABSTRACT

Described here is a balloon catheter system, a very low profile medical device system having one or more adjustable length and/or adjustable diameter components, (e.g., balloons), system accessories, and system components. Also described are methods for using the variations of the system and its parts, such as by performing procedures, such as dilatation and other methods clear from the description, and for placing implants such as stents or occlusive members into tubular organs, open regions of the body, and other body sites. The diameter and effective length of the implanted stents may, in some variations, be chosen during the procedure without removing the device, or any of its constituent parts, from the patient typically by expanding or inflating a member perhaps in combination with restraint of at least some portion of an expandable member. The system generally includes either or both of: a.) a balloon catheter having at least one balloon, generally distally located, and b.) at least one balloon integral with a guide member, which balloons are adjustable in length and optionally in diameter. The system may be used to introduce and to deploy implants of types such as those that maintain the patency of an open anatomical structure, install a graft, occlude a selected volume, isolate a region, treat a region in a lumen with a surgical procedure or medicinal materials, or collect other (desirable or undesirable) occlusive members at a site.

### RELATED DOCUMENTS

Benefit is claimed under 35 USC 119 or 120, as appropriate, from each of the following:
a.) a provisional application filed on Sep. 5, 2003 entitled “A Stent Delivery Topology” (Ser. No. 60/500,248) by Tuvia Dror Kutscher and Doron Marco,
b.) a provisional application filed on Oct. 14, 2003 entitled “An Improved PCI Deployment Procedure” (Ser. No. 60/510,442) by Tuvia Dror Kutscher and Doron Marco,
c.) a provisional application filed on Oct. 22, 2003 entitled “An Improved PCI Deployment Procedure II” (Ser. No. 60/512,864) by Doron Marco and Tuvia Dror Kutscher,
d.) a provisional application filed on Nov. 12, 2003 entitled “Multi Length and Diameter Angioplasty Balloon” (Ser. No. 60/518,632) by Doron Marco and Tuvia Dror Kutscher,
e.) a provisional application filed on Nov. 24, 2003 entitled “Very Low Profile Medical Device System Having An Adjustable-Length Balloon” (Ser. No. 60/524,026) by Doron Marco and Tuvia Dror Kutscher,
g.) a U.S. patent application filed on Dec. 19, 2003 entitled “Very Low Profile Medical Device System Having An Adjustable Balloon” (Ser. No. 10/741,927) by Doron Marco and Tuvia Dror Kutscher,
f.) a provisional application filed on Feb. 27, 2004 entitled “Balloon Catheter Assembly Having An Adjustable Length Balloon” (Ser. No. 60/548,397), by Doron Marco and Tuvia Dror Kutscher,
h.) a provisional application filed on Apr. 3, 2004 entitled “Balloon Catheter Assembly Having An Adjustable Length Balloon,” (Ser. No. 60/559,106) by Tuvia Dror Kutscher and Doron Marco,
i.) a provisional application filed on May 27, 2004 entitled “Balloon Catheter Assembly Having An Adjustable Length Balloon (III),” (Ser. No. 60/575,718) by Doron Marco and Tuvia Dror Kutscher, and
j.) a provisional application filed on Jul. 7, 2004 entitled “Balloon Assembly (IV)” (Ser. No. 60/586,147) by Doron Marco and Tuvia Dror Kutscher.

Each of these provisional applications and utility applications is incorporated by reference for all purposes.

### FIELD

Described here is a balloon catheter system and a fine (or very low profile) medical device system, in each case having an expandable member that may be controllably expanded to various lengths or diameters without removal from the human body. Such controllable expansion permits use of the described systems to implant stents at diameters and with lengths that may be chosen while a system and the stent is in the human body and to effect those chosen diameters without removing the stent from the body. These expandable systems may also be used to providing controllable, sized cutting balloons. The expandable member is configured to allow such controllable expansion, for instance, by utilizing one or more expandable members, e.g., inflatable balloons, and expansion-limiting sleeves. Described in conjunction with the system are inflatable members having one or more adjustable length and/or adjustable diameter balloons, system accessories, and system components. Also described are methods for using the variations of the system and its parts, such as by performing procedures, such as dilatation and other methods clear from the description, and for placing implants such as stents or occlusive members into tubular organs, open regions of the body, and other body sites. The diameter and effective length of the implanted stents may, in many variations, be chosen during the procedure without removing the device, or any of its constituent parts, from the patient. The system may include at least one controllably expandable member often found in either or both of: a.) a balloon catheter having at least one balloon, generally distally located, and b.) at least one balloon integral with a guide member, which balloons are adjustable in length and optionally in diameter. The system may be used to introduce and to deploy implants of types such as those that maintain the patency of an open anatomical structure, install a graft, occlude a selected volume, isolate a region, treat a region in a lumen with a surgical procedure or medicinal material, or collect other (desirable or undesirable) occlusive members at a site.

### BACKGROUND

Implants such as stents and occlusive coils have been used in patients for a wide variety of reasons. For instance, stents are used to treat arterial stenosis secondary to atherosclerosis. Various stent designs have been developed and used clinically, but self-expandable and balloon-expandable stent systems and their related deployment techniques are now predominant. Examples of self-expandable stents currently in use are WALLSTENT® stents (Schneider Peripheral Division, Minneapolis, Minn.) and Gianturco stents (Cook, Inc., Bloomington, Ind.). More commonly used balloon-expandable stents include the CYPHER® and PALMAZ® stents (Cordis Corporation, Warren, N.J.) and the TAXUS® stent (Boston Scientific Corporation, Boston, Mass.).

Typically, either during or after a balloon angioplasty, a self-expandable or balloon-expandable stent is advanced to the target site and expanded or implanted. A protective sheath or membrane may be retracted to allow expansion of a self-expanding stent or a delivery balloon may be inflated to expand the stent.

The physician typically selects the size of the deployed stent, both length and diameter, in a number of stages—the first being the selection of a range of sizes for ready access during the later deployment procedure from a clinical review of the patient and the patient's condition. The final decision is often made during the procedure. The availability of a number of stent sizes and lengths is often the most critical of factors in the success of that procedure. In most current instances, the diameter of the deployed stent is determined by the diameter of the non-compliant balloon used to expand the stent. A decision to change stent diameter is also a decision to change deployment balloons. The outer balloon catheter must be removed; the stents cannot be changed in vivo.

Smaller diameter or lower profile implant deployment devices that release an implant in a more precise, continuous or step-wise fashion, and those that allow choice of stent size (both length and diameter) without removal of the deployment device from the body would be of significant medical value.

### SUMMARY

Described here is medical device comprising either or both of: a.) a balloon catheter having at least one balloon, generally distally located, and b.) a low profile, balloon-device-containing system, that includes an adjustable-length or diameter balloon. The variations of the system may be used for implant delivery, intraluminal implant reforming or retrieval, and various surgical and medical treatment procedures. It may be based upon either or both of: a.) a balloon catheter, e.g., a catheter having at least one balloon, generally distally located, as might be used in angioplasty or stent placement procedures, and b.) a core guide or guide member, e.g., a guidewire-like component, that is, a component integral with an expandable member, e.g., a balloon, having a flexibility and size such that the guide member and the integrated balloon are able, for instance, to reach a selected treatment site in the cardiovasculature or the neurovasculature without the requirement of using either a catheter exterior to the device or a guidewire interior to the device for the last two inches of access.

The system may comprise a remotely directable core guide member comprising in turn, one or more adjustable-length balloons, the core guide member being variously directable from outside the patient's body and having at least one balloon being adjustable at least in length or diameter from outside the body while the core guide member is also inside the body. The core guide member has a proximal end and a distal end. Generally near the distal end, the guide member includes an inflatable balloon member that is adjustable in length and/or in diameter. The system may also be configured to utilize, in addition to, or in place of, a balloon catheter as may be currently used in treating various arterial stenoses or the other balloon catheters described elsewhere in this document. The balloon catheter so-used may include several balloons, adjustable in diameter or in effective length or both.

The variations of the system may be configured to direct at least one implant having an exterior and interior surface to an anatomical treatment site by the remote manipulation of a user.

The system may be used in lumens of tubular organs such as blood vessels, (e.g., arteries and veins including variously small and large vessels, intracranial vessels, peripheral vessels, adjacent aneurysms, arteriovenous malformations, arteriovenous fistulas, etc.), ureters, fallopian tubes, cardiac chambers, ducts such as bile ducts and mammary ducts, large and small airways, and hollow organs, e.g., stomach, intestines, and bladders.

The deployed implant may be of a design that is of a size that is smaller prior to and during delivery and then larger after implantation. The implant design may be used to provide or to maintain patency in an open region of an anatomical structure, or to occlude a site, or to isolate a region (e.g., to close an aneurysm by blocking the aneurysm opening or neck by placement of an implant in an adjacent anatomical structure such as an artery or gastrointestinal tubular member), or to hold a number of occlusive devices (e.g., coils, polymeric masses, or hydratable polymeric noodles) or compositions at a site to be occluded or supported. The implant design may be one that collects embolic material in a blood stream. The implant design may be used to maintain a body lumen, e.g., a cardiac artery, in an open condition. The system may also be employed for implant delivery, with or without local drug delivery, into solid organs or tissues including skin, muscle, fat, brain, liver, kidneys, spleen, and benign and malignant tumors.

### BRIEF DESCRIPTION OF THE DRAWINGS

FIG. 1A is a plan view of the described low profile, adjustable-length balloon-device-containing system showing various of its components and system tools that may be used by other system variations.

FIG. 1B is a plan view of one variation of the described system including an adjustable-length balloon with multiple stacked balloons.

FIG. 1C is a plan view of another variation of the described system including various of the structures suitable for adjusting the length or the diameter of an implanted stent, and specifically showing one variation of an exchange system.

FIG. 2A is a plan view of the core guide member.

FIGS. 2B and 2C are partial cross-sectional views of the distal end of core guide member.

FIGS. 2D to 2I are cross-sectional views of variations of the core guide member.

FIGS. 3A and 3B are cross-sectional side views of variations of the distal end of the core guide.

FIGS. 4A, 4B, and 4C are cross-sectional side views of distal end variations of the core guide showing open passageways.

FIGS. 5A, 5B, and 5C show a variation of the described system utilizing a balloon catheter in conjunction with one or more of a constraint member and a core member with associated balloon. FIG. 5C is a partial cross-sectional side view of a variation having a balloon catheter, a core guide member having a distally located balloon, and a constraint member.

FIG. 6A is a partial cross-sectional side view of a variation having a balloon catheter with a small distal balloon and a constraint member.

FIG. 6B is a cross-sectional side view of one variation of the FIG. 6A a balloon catheter.

FIG. 6C is a cross-sectional diametric view of the FIG. 6B balloon catheter.

FIG. 6D is a cross-sectional side view of another variation of the FIG. 6A balloon catheter.

FIGS. 7A-7C show various balloon catheter profiles.

FIGS. 8A and 8B are cross-sectional side views of the core guide with a constraint member in place and the integral balloon being expanded.

FIG. 9A is a longitudinal cross-sectional view of the distal end of a constraint member variation.

FIG. 9B shows the longitudinal cross-sectional view of the FIG. 9A constraint member variation and its placement with respect to the uninflated balloon.

FIGS. 10A and 10B are, respectively, a longitudinal cross-sectional view and a radial cross-sectional view of the distal end of another constraint member variation.

FIGS. 10C and 10D show a longitudinal cross-sectional view of the FIG. 10A constraint member variation and its placement with respect to the uninflated balloon and to the inflated balloon.

FIGS. 11A and 11B are, respectively, a longitudinal cross-sectional view and a radial cross-sectional view of the distal end of a multi-balloon guide member variation.

FIGS. 12A, 12B, and 12C show, respectively, a partial cross-section, side-view and a cross-sectional view and a partial cross-sectional view of a multiple balloon, balloon catheter using a movable plug wire for release of inflation fluid into the surrounding body lumen.

FIG. 13A shows a cross-section of a stacked multi-balloon layer and an outer elastic member. FIG. 13B shows the further re-formation of FIG. 13A outer balloon by the constraining member.

FIGS. 14A to 14B show variations of an assembled core guide member having a number of lumens that may be used in a multi-balloon device such as shown in FIGS. 11A and 11B.

FIGS. 14C and 14D show cross-sectional and side-cross sectional views of further variation of a multi-passageway core guide member having multiple interior tubes with passageways.

FIGS. 15A, 15B, 15C, and 15D show cross-sections of multiple balloon, concentric tubing, balloon catheters.

FIGS. 16A, 16C, and 16E show cross-sectional views of multiple balloon, balloon catheters with various inflation fluid lumen configurations. FIGS. 16B, 16D, and 16F show longitudinal cross-sections of the FIG. 16A, 16C, and 16E catheters.

FIG. 17A shows a partial, longitudinal, cross-section of a multiple balloon, balloon catheter with tubular inflation fluid members. FIGS. 17B, 17C, and 17D shows cross-sections of the FIG. 17A catheter.

FIGS. 18A, 18B, 18C, 18D, 18E, and 18F show additional assisted folding balloon structures.

FIGS. 19A, 19B, and 19C show another variation of the described system including a balloon catheter in, respectively, a side section and two separate cross-sections.

FIGS. 20A, 20B, 20C. and 20D show cross-sections of various multiple balloon, balloon catheters.

FIGS. 21A, 21B, 21C, and 21D show side view or longitudinal sections of balloon catheters having stents mounted on the balloons and that include manually placed exterior sheaths situated to prevent expansion of some section or portion of the mounted stents.

FIGS. 22A-22H show partial cutaway, side view depictions of a number of elastic deflation aid variations.

FIGS. 23A to 23D show partial cross-sectional views of the steps of inflating a highly compliant balloon.

FIGS. 24A, 24B, and 24C show partial cross-sectional views of a constraint member having a distal inflation control section during the steps of inflating a highly compliant balloon.

FIGS. 25A and 25B show partial cross-sectional views of balloons having integral distal and proximal constraint members during the steps of inflating a highly compliant balloon.

FIGS. 26A, 27A, 28A, and 29A show partial cross-sectional views of ends of various constraint member inflation control sections with the balloon uninflated and FIGS. 26B, 27B, 28B, and 29B show the sections with the balloon inflated.

FIGS. 30, 31, and 32 show, respectively, a side view of a variation of an implant delivery component with a single large sector and having multiple implants with similar or the same sizes, a side view of another variation of an implant delivery component with a single large sector and multiple implants with differing sizes, and an exploded view of a variation of an implant delivery component assembled from smaller sectors of the type shown in FIGS. 30 and 31.

FIGS. 32A and 32B show, respectively, a partial cross-sectional side view of an implant delivery sleeve and a cross-sectional view of the wire comprising that sleeve.

FIGS. 33A, 33B, and 33C show, respectively, a partial cross-sectional side view of another variation of the described implant delivery sleeve and two cross-sectional views of the ribbons comprising that sleeve.

FIGS. 34A and 34B show, respectively, non-inflated and inflated exterior views of an implant delivery sleeve assembly with an elastic sleeve and a number of stents mounted thereon.

FIGS. 35A and 35B show, respectively, a partial cross-sectional side view of an implant delivery sleeve with a self-expanding stent and a cross-sectional view of the self-expanding stent with the restraining cover removed.

FIGS. 36A-36C show the procedure for inflating a single implant or stent mounted initially on the implant delivery component.

FIG. 37 shows a partial-cutaway side view of a stent-graft delivery component.

FIG. 38 shows a cross section of a balloon catheter with a stent delivery sleeve.

FIG. 39 shows a cutaway side view of a direct stenting variation.

FIG. 40 shows a number of stents mounted directly on a multiple balloon structure.

FIG. 41 shows stents mounted directly on a balloon catheter having a smaller single distal balloon and a multiple, stacked, proximal balloon structure or section.

FIG. 42 shows a side view, cross-sectional view of a number of interlocking stent members mounted on a stent delivery sleeve.

FIG. 43 shows a side view, cross-sectional view of a tapered balloon having a stent mounted on it.

FIG. 44A shows a side view, cross-section of a stacked, multi-balloon, stenotic cutting balloon catheter. FIGS. 44B and 44C show, respectively, side-view, cross-sections of a stenotic cutting blade before the balloon is inflated and after the balloon is inflated.

FIG. 45A shows a partial cutaway, side view of a tool used for cutting stenoses. FIG. 45B shows a partial longitudinal cross-sectional view of the FIG. 45A component and FIG. 45C shows the operation of the FIG. 45A tool using one of our inflatable balloon devices.

FIG. 45D shows a partial longitudinal cross-sectional view of the FIG. 45A component with a multi-balloon catheter within it.

FIG. 46A shows a partial side view of a forming tool used for shaping stenoses or limiting the expansion of the balloon to a specific region of a lumen. FIG. 46B shows a partial longitudinal cross-sectional view of the FIG. 46A tool. FIG. 46C shows the operation of the FIG. 46A tool in a partial longitudinal cross-sectional view while using one of our inflatable balloon devices. FIG. 46D shows the operation of the tool in a top view.

FIGS. 47A to 47C show longitudinal cross-sectional views of a multiple caul forming component.

FIG. 48 shows a longitudinal, sectional view of a conical-shaped caul forming component.

FIGS. 49A and 49B show a partial side view and a partial longitudinal cross-sectional view of a drug delivery component.

FIGS. 50A, 50B, and 50C show partial side view, partial longitudinal cross-sectional views of rapid-exchange variations.

FIGS. 51A-51N show the steps of using the described system.

FIGS. 52A and 52B show cutaway views of steps of using the described system to close an occluded aneurysm.

FIGS. 53A, 53B, and 53C show cutaway views of steps of using the described system to reform a kinked stent in a bend in an artery.

FIGS. 54A-54J show a procedure for using the described system to reform a kinked stent in an artery.

FIGS. 55A to 55C, 55D1-55D3, 55E1-55E3, and 55F show cutaway views of a procedure for using the described system to implant a stent in a bend in an artery.

FIGS. 56A-56E show a procedure for using the described system to dilate a severely stenosed lesion in an artery.

FIGS. 57A to 57E show cutaway views of a procedure for using the described system to directly implant a stent in an artery, a procedure often described as direct stenting.

FIG. 58A shows a partial side view, partial longitudinal cross-sectional view of a multi-layer balloon catheter having separate lumen supplies for each balloon. FIG. 58B shows the fill line and valve openings for inflating the inner two balloons. FIGS. 59A and 59B show the valve openings for inflating the inner two balloons, one by one.

FIG. 60 provides a schematic representation of an easy-to-use valving arrangement for safely inflating and deflating multiple balloons on a stacked balloon catheter.

FIGS. 61A-61J provide a step-by-step method for forming a stacked balloon section.

FIGS. 62A-62C show respectively, a side sections, a top view, and a side section of a rapid exchange system in conjunction with the tubular members of the system, e.g., the constraining member.

FIGS. 63A-63D show side, sectional views of the structure of a multi-balloon catheter structure and steps for inflating it.

FIGS. 64-67 show kits including various systems and components described herein.

### DETAILED DESCRIPTION

Described here are devices, systems, and methods for delivering implants into both open and solid regions of the body. The term “region” as used herein refers to luminal structures as well as solid organs and solid tissues of the body, whether in their diseased or non-diseased state. Examples of luminal structures include, but are not limited to, blood vessels, arteriovenous malformations, aneurysms, arteriovenous fistulas, cardiac chambers, ducts such as bile ducts and mammary ducts, fallopian tubes, ureters, large and small airways, and hollow organs, e.g., stomach, intestines, and bladder. Solid organs or tissues include skin, muscle, fat, brain, liver, kidneys, spleen, and benign and malignant tumors.

Additionally, we use the term “expandable” to include a passive ability to change and to increase in size. The term is meant to include the more narrow term “inflatable” where a fluid, i.e., a gas or liquid, is used for expansion under pressure.

Specifically described is a system including one or more balloons or inflatable members, perhaps stacked radially or longitudinally displaced from each other, one or more of which may be adjustable in length or diameter, particularly after placement in the human body. This “diameter” adjustability is in addition to the mere inflation of the balloon and will be discussed in additional detail below. The system often has a significantly low profile, e.g., in one variation, the balloon is mounted to a small diameter core member or guide member that is otherwise similar in size and function to a guidewire used in a specific body region, such as the neurovasculature. Said another way: in many variations of the system, the core member or guide member is a multifunctional component that is able to function in much the same way as is both the guidewire and the catheter in more conventional guidewire/balloon catheter systems. In addition, the system may include a balloon catheter with a balloon section that is inflatable and deflatable. That balloon section may be multi-layered, e.g., comprising multiple layers of balloons located radially adjacent each other and typically independently inflatable. Also described are various complementary implants, components, and tools suitable for use with the balloon and its integrated system, kits of complementary components, and procedures for using the devices. The described system and its various components, where specified, are of a size and flexibility that are suitable for use in the small confines of the neurovasculature. Of course, since they are useful in the narrow regions of the neurovasculature, they will be similarly suitable for those portions of the body having openings that are not as confining.

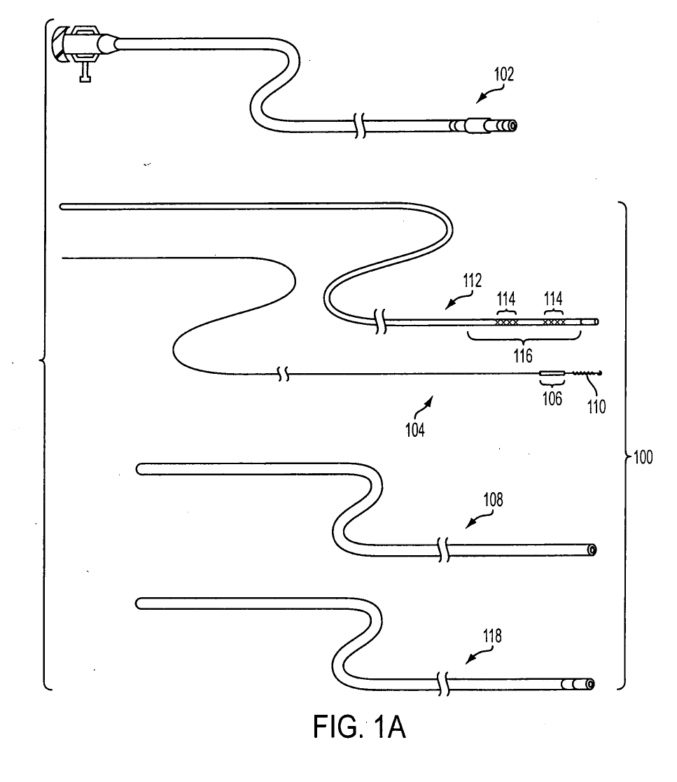

FIG. 1A depicts a variation of a typical system (100) as described here, of the type using a core guide member. The Figure also shows certain of its major subcomponents and accessories as well as an optional balloon catheter (102). A core guide member (104) having an inflatable member or balloon (106) located generally distally on the core guide member (104) is shown. The balloon (106), in cooperation with a slidable constraining member (108) that fits over core guide member (104), is adjustable in situ, particularly after the balloon is situated at a selected position in the human body. The core guide member (104) includes a passageway or lumen that is in fluid communication with an inflation region beneath the balloon (106) and is the pathway by which fluid is introduced into the balloon (106) for inflation. Also shown at the distal end of core guide (104) is a guide tip (110). The guide tip (110) typically is radio-opaque, is usually functionally fairly soft and compliant to allow easy and non-traumatic passage through a body lumen, and is pre-formable by the user to allow selection of branching passageways in the body upon rotational manipulation of the device. The guide tip (110) is often made of a flexible, helical coil and an interior ribbon tip. Also shown in FIG. 1A is one variation of an implant delivery component (112) that is, in this instance, a delivery component for independently delivering stenting devices (114) from a delivery sleeve (116). The stent delivery component (112) may be sized to slide onto the exterior of the constraining member (108) and cooperate with the balloon (106) to permit such independent stent delivery. In some variations discussed below, the constraining member (108) may instead be sized to be situated exterior to the stent delivery components. Multiple stents (114) may be mounted on the sleeve (116) and delivered variously one-by-one, all together, or in multiples in each instance without withdrawing the delivery component (112) from the body.

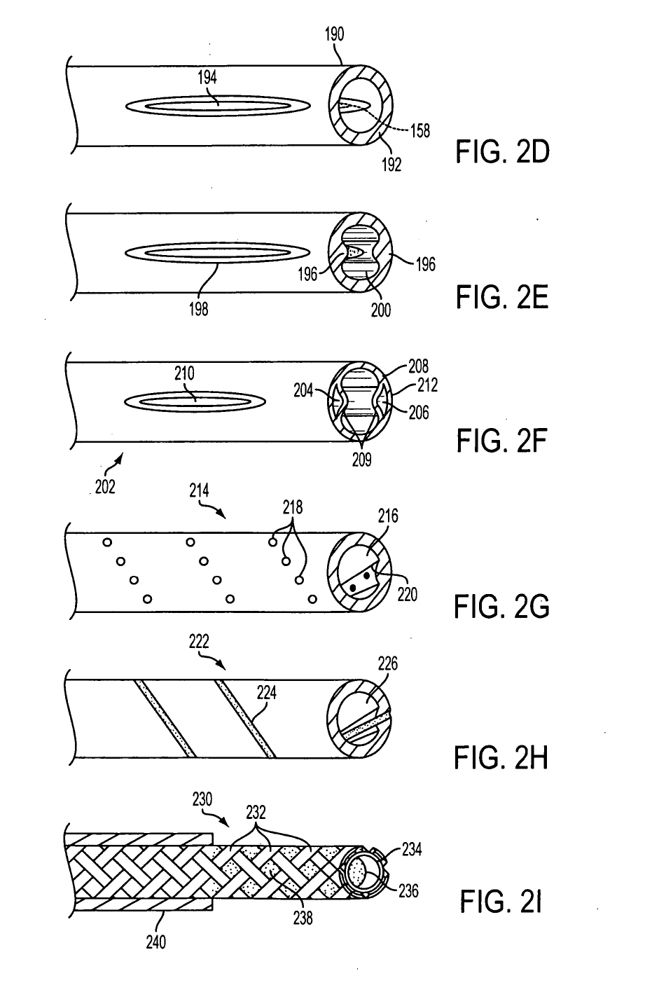

Finally, FIG. 1A shows a generic tool or accessory (118)—several variations of which are explained in more detail below—variously to fit on the exterior of the implant delivery component (112) or to fit in place of the implant delivery component (112) upon constraining member (108) or to fit upon a balloon catheter (103), said balloon catheter as shown, for instance, in FIGS. 5A and 5B.

Alternatively, the stent delivery component (112) may be sized in such a way to slide directly onto the exterior of core guide member (104) whilst the constraining member (108) is sized to fit over the stent delivery component (112).

FIG. 1B shows one variation of another typical system (120) that utilizes a balloon catheter (122) having multiple, radially stacked balloons (124) and an optional, distally located balloon (126). The various balloons (124, 126) typically are each independently inflatable using a variety of inflation strategies. Specifically, each balloon in the balloon stack (124) and the distal balloon (109) are independently inflatable. Depicted in the FIG. 1B is a version in which several independent tubing members (128) are used to feed inflation fluid to the balloons, singly or in parallel, using a control valve (130) operated by the physician-user. The tubing members (128) used to fill the balloons (124, 126) for, e.g., angioplasty or stenting, are also normally used to deflate the balloons (124, 126) at the conclusion of the procedure. The inflation fluid may flow through the control valve (130) for disposal (132) perhaps after passing through a release or control valve (134) also operated by the user.

In this variation of the balloon catheter based system, the catheter shaft (136) comprises a larger diameter metallic tubing (138), a smaller diameter metallic tubing (140), and a polymeric section (142), all joined to include at least a common lumen for a guidewire (144) that may be used in the normal way. It should be noted that tubing members (128) are-shown passing from the interior of metallic sections (138, 140) near the distal end of catheter section (140) to access the balloons (124) outside of the guidewire lumen in catheter section (142). This will be explained in more detail below and is one variation for providing inflation fluids to those balloons.

Also shown in this FIG. 1B is the placement of a number of stents (146) that may be used in cardiac procedures (and in other procedures) for “direct stenting,” i.e., placing a stent without necessarily performing a prior angioplasty step. Inclusion of these stents (146) on the multi-layer balloon (124) is optional, as will be explained below.

FIG. 1C shows another variation of the system (150) that utilizes a balloon catheter (152) having multiple, radially stacked balloons (154) and an optional, distally located balloon (156). As was the case with the system found in FIG. 1B, the various balloons (154, 156) typically are each independently inflatable using a variety of inflation and constraint strategies. Again, each balloon in the balloon stack (154) and the distal balloon (156) are independently inflatable. Depicted in the FIG. 1C is a version in which a control valve (160), operated by the physician-user, is used to feed inflation fluid to the balloons.

In the variation of the balloon catheter based system shown in FIG. 1C, balloon catheter (152) includes an opening (162) proximally of balloon stack (154) allowing passage of a rapid exchange wire (158) through the catheter wall into the catheter lumen and eventually out the distal end of the catheter (152). The guide catheter (164) is shown, as is the user's control valve (160) allowing control of the deployed stent diameter merely by turning the control valve to a specific position.

Core Guide Member

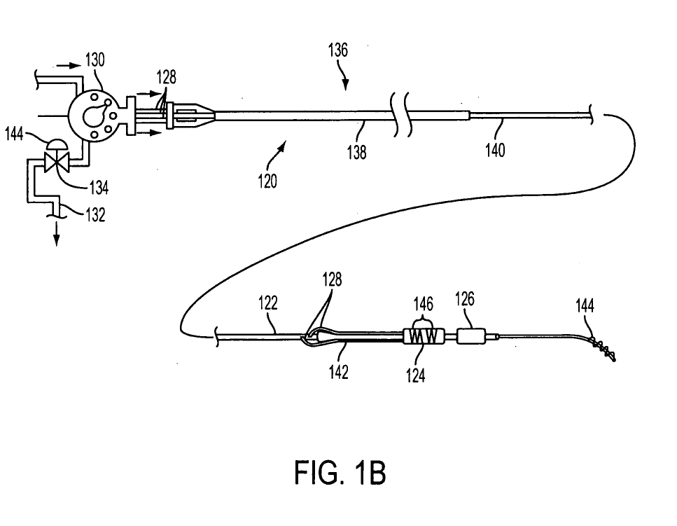

FIG. 2A shows a schematic view of a typical core guide member (170) and depicts various components and features of such a typical core guide member (170). As noted above, a core guide member is a device having the characteristics of a guide wire but that also may be used in place of a balloon catheter in certain circumstances or may be used to perform the functions of a guide-wire in conjunction with a balloon catheter or other catheter. The core guide member, as generally depicted in FIG. 1A, is made up of a number of components, in its most simple variation: a core guide member body and a balloon section. The “constraining member,” the component effecting balloon length control in many variations of both the described core-guide-member-containing system and the balloon-catheter-containing system, is also described below.

Returning to FIG. 2A, the body (132) of core guide member (130) includes at least one passageway or lumen (174) usually passing from the proximal end (176) of core guide member (170) to the distal end (178). The passageway (174) is in open fluid communication with one or more balloon inflation openings (180) (here shown as a slot in the distal region) and, is open to at least one inflation area (182). The inflation area (182) is the region found beneath a balloon as may be mounted in a fashion shown in more detail below. It should be noted that this depiction shows but a single lumen or passageway (174) and a single balloon inflation opening (180), but as will be shown below, the core guide member may involve multiple, e.g., more than one, core passageways with multiple balloon inflation ports or openings each independently available to inflate separate balloon members. A guide tip (184) is shown in the depiction of FIG. 2A. Various designs and styles of guide tips may be used in this core guide member (170). They may correspond to designs used in those guidewires currently known or used for specific purposes and body areas.

The core guide member body (172) may be made of any of a wide variety of materials that are suitable for a device of this type of chosen medical service. That is to say, the core guide member body (172) may be comprised of neat metallic alloys, metals, polymers, or may be an assemblage or composite. For instance, suitable alloys include the group known as “superelastic alloys”, appropriate stainless steels, various engineering polymers optionally containing fibrous reinforcing materials, woven or wound assemblages of these materials, and others that generally meet the criteria of the ability to serve as a guidewire and, optionally, be substantially non-kinking in such service. Examples of suitable superelastic alloys include nickel titanium alloys (e.g., 48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Widely used NiTi alloys, generally known as “nitinol,” are those described in U.S. Pat. Nos. 3,174,851; 3,351,463; and 3,753,700, each of which is hereby incorporated by reference. Such an alloy tolerates significant flexing even when drawn as a very small diameter wire. The formation of medical devices from nitinol alloys having both superelastic and shape memory properties is well known in the art, and described in U.S. Pat. Nos. 4,795,458 and 5,037,427, and PCT publication WO 94/16629, each of which is incorporated by reference. Other superelastic materials such as those described by Saito, et al. in SCIENCE, 300, 464-467 (2003) of titanium, zirconium, vanadium, niobium, and tantalum together with a small amount of oxygen, seem also to be appropriate materials. Anti-kinking facilities may be enhanced by wrapping a tubing of such a material with, e.g., a braided or coiled exterior layer. Some of these variations are shown in more detail below. In variations of the system where the core guide member body (172) is intricate, e.g., multi-lumened, formation of the core guide member may most easily be had via polymer extrusion. Various polyimides are suitable as relatively strong but stiff materials for the shaft of the core guide member body (172).

In some variations of the system, the designer may perceive a need to provide a higher level of torqueability to or of stiffness to the proximal end of the core guide member body (172), particularly when the body is polymeric. In such instances, some portion of the proximal section of the core guide member body (172) may be formed using metallic tubing to reinforce the body, e.g., by placement of the metallic tubing outside and perhaps glued or otherwise sealed to the inner portion. Indeed, in some variations, the proximal portion of the core guide member may comprise one or more metallic tubing members. The use of various braids or coils wrapped or otherwise situated around the core body to reinforce the more proximal section of the core guide member body (172) is useful. The core guide member body (172) may be initially formed, e.g., by coextrusion with a braid or coil placed interior to the body wall for at least a portion of the body length. As will be discussed below, relating to another version of the system, the system including a balloon catheter, the catheter body may be similarly be constructed, where, for instance, the proximal portion of the catheter may comprise one or more sections of metallic hypotube.

The core guide member (172) may be of a constant diameter or may be tapered with the smaller end of the taper towards the distal end of the body.

FIGS. 2B and 2C show structures for “fairing” the balloon in the region of the inflation region and show the placement of the balloon over one variation of the inflation opening.

FIG. 2B shows a generally distal section (178) of the core guide member including a core guide member body (172) with an opening (180) from passageway (174) to an inflation area beneath balloon (184). Balloon (184) is placed in sealing contact with core guide member body (172) by a pair of regions (186) that adhere to the core guide member body (172). The adherence may be by use of adhesives, glues, heat, solvent welding, welding, heat shrinking, etc. Adjacent the distal and proximal ends of balloon (184) are a pair of locator coils (188) proximally and (190) distally. These coils help to “fair in” the balloon, to provide a generally constant diameter surface, to often provide a radio-opaque marker, and to permit the core guide to pass easily through the selected region in the body. An additional filler (192) is also shown in FIG. 2B as a layer that provides a measure of constant diameter proximally of proximal coil (188).

FIG. 2C shows a cross section of a variation similar to that shown in FIG. 2B excepting that the optional proximal and distal locater coils (188, 190) are not used in the particular variation. A fairing covering (194, 196) is shown proximally and distally of the balloon member (184).

FIGS. 2D-21 show examples of suitable core guide member bodies and the inflation openings suitable for the described system.

FIG. 2D shows a core guide member having a generally tubular body (190) and a generally constant wall thickness (192). Also shown are a pair of openings, in this case, slots (194), that allow communication from the inner core passageway into the balloon that eventually will be situated upon the inflation area of this body (190).

FIG. 2E shows a similar configuration however with the exception that a pair of ribs (196) are found in the body passageway and, in this example, are shown to be placed in such a fashion that the openings (198) passing from the passageway (200) of the core guide member body to the exterior of the body pass through the ribs. In this way, the ribs are able to provide an additional measure of stiffness in the region of the fluid openings (198) and lessen the tendency for bending, rupture, or kinking of the body during tight maneuvering in the body.

The slots shown in FIGS. 2D and 2E used as openings (194, 198) respectively provide, in some instances, enhancement to the operation of the device. Specifically, because of the lengths of those slots, when high pressure fluid is applied to the inside of the core guide member body's passageway, the slots will expand, or may expand depending upon the physical size of the device, to allow larger amounts of fluid to pass into the balloon and to allow higher rates of operation of the balloon during the expanding and deflating steps. With a proper selection of materials, the slots will return to their normal positions once the fluid flow to or from the balloon is terminated. Proper sizing of the slots will also allow enhancement of the fluid's return flow rate.

It should be noted that the interior ribs (196) found in FIG. 2E need not extend the entire length of the body to gather whatever benefits may accrue through their presence. For instance, the ribs may be placed only in the neighborhood of the slotted opening to provide enhanced strength or springiness in that area.

FIG. 2F shows a variation (202) of the core guide member body in which two lumens or passageways (204) and (206) are shown to be exterior to the central core passageway (208). As is the case with the other variations, the opening into the inflation region (not shown) is accomplished by a slot (210) allowing fluid communication between the lumen and the balloon. This variation provides for two passageways that may be discretely operated by passage of fluid independently of each other or they may be operated together with concurrent flow of fluid and passage of that fluid through the openings (210) into one or more balloons. The number of passageways and the number of flutes (209) extending into the central core (208) is dependant upon of the needs of the designer for the particular medical device designed. The outer periphery of this particular design includes an outer covering (212) that cooperates with the inner core (208) to form the inflation passageways (204, 206). The wall or covering (212) surrounding the central core passageway (208) may be of the materials mentioned above, e.g., high flexibility stainless steels, superelastic alloys, and certain engineering plastics, perhaps fiber filled. The outer covering (212) typically would be a tubular polymeric member reasonably resistant to stretching upon application of fluid pressure to the various passageways (204, 206).

FIG. 2G shows another variation of the core guide member body (214) having a passageway (216) and one or more holes or orifices (218) between passageway (216) and the exterior of the core guide member body (214). An optional rib (220) provides strength and stiffness in the region where the holes pass through the wall. This rib is optional. The holes (218) are shown as being placed in a spiral formation. Although this formation has some benefits, it need not be used. The holes also need not be of the same size but may be varied to fit the situation as the device designer so desires.

FIG. 2H shows another variation of the core guide member body (222) having a helical slot (224) for fluid passageway from the interior passageway (226) to the exterior of the body (222).

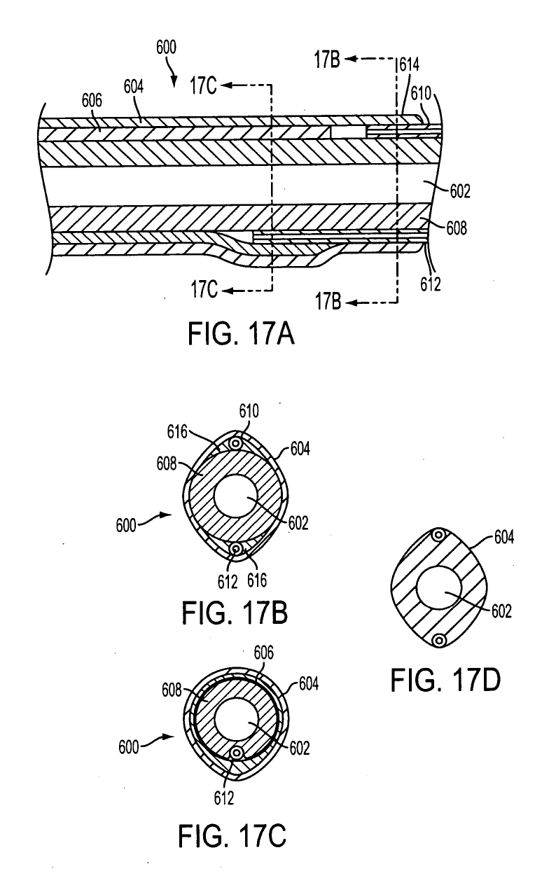

FIG. 21 shows a variation of the core guide member body (230) which is formed of a braided ribbon or ribbons (232) and having an interrupted liner (234) in passageway (236), the interruption allowing for fluid flow to the balloon. The opening (238) between adjacent ribbons (232) are those used to inflate the balloon in the inflation area. An exterior tubing (240) may also be used to provide smoothness and the like to the device. The braided core guide member body (230) may be used where the guide member must be extremely flexible and very kink-resistant. The braids would likely be a metal, alloy, or polymer of a specific type, such as the super-elastic alloys, various stainless steels, and a few polymers (such as “engineering plastics”.)

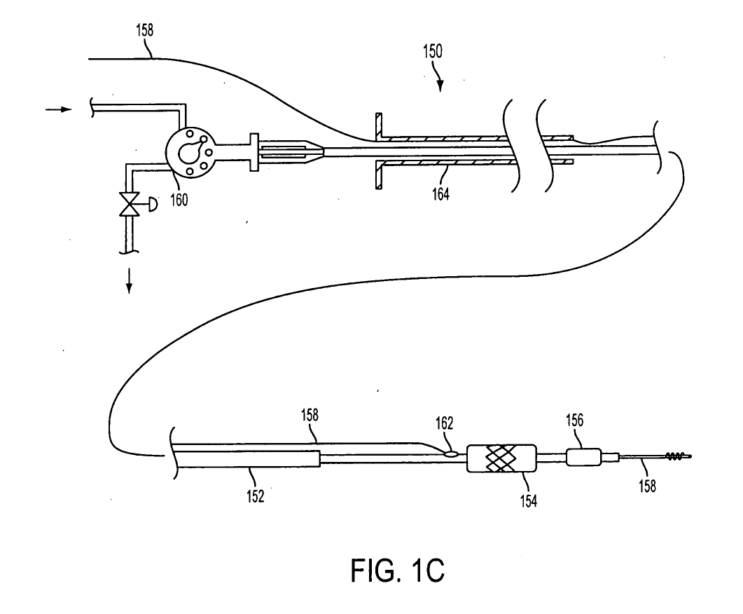

FIG. 3A shows a side view, cutaway depiction of a distal end of a core guide member (240) with a balloon (242) placed over the inflation area (244). The distal most end of the body (246) is closed with a plug (248). A core tip (250) made up of tapering helical coil (252) is also shown. The distal ribbon (254) (or, alternatively, a wire) is shown to be embedded in the closing block (248). The distal-most tip (256) is often a “glob” of a slippery polymer or maybe the result of heating the end of the coil in a melting torch. Although the coil (252) is shown to be tapering, of course, it need not be tapered.

The balloon (242) shown in FIG. 3A is adhesively and sealingly attached to the core guide member body using suitable adhesives or other “adhering” methods. Another method for securing the end of balloon (242) is shown in FIG. 3B. In that variation an outer covering (258) for perhaps with a cinch or band (260) securing the end of balloon (242) alone or together with adhesives to the guide member body (262). Even in those instances as may be described elsewhere herein, where the balloon expansion fluids may be allowed to seep or otherwise exit the volume contained by the balloon and enter, e.g., the vasculature, the fact that the balloon is otherwise in direct contact with the core guide member or catheter in a way that allows inflation, the balloon or inflatable member will properly be described as “sealingly attached.”

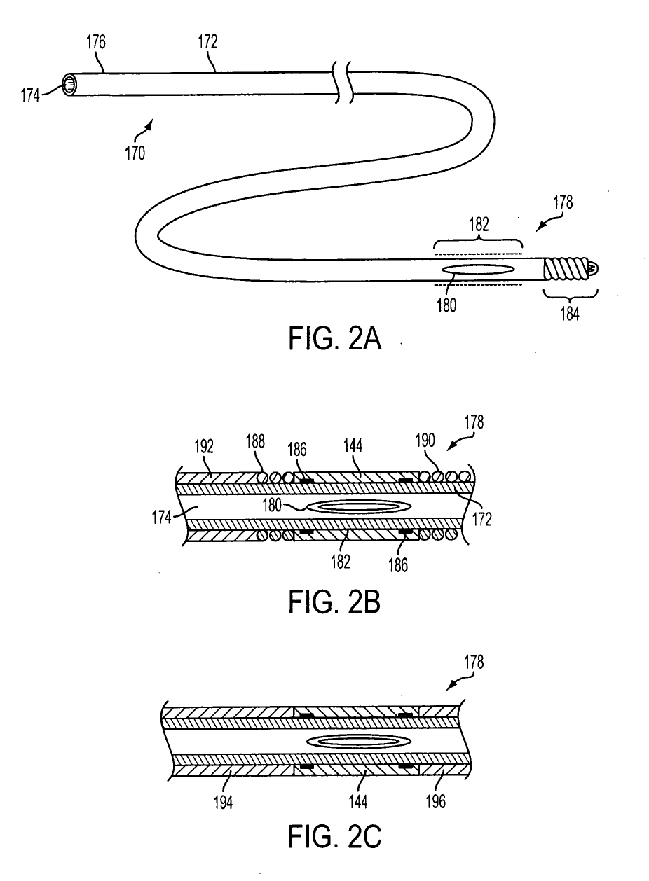

The distal end of the core guide member is referred to as “closed” or “closable”. One variation of the “closed” end is shown variously in FIG. 3A and with more particularity in FIG. 4A where a plug (248) closes the closable end of the core guide member (262). However, the balloon member (242) is situated in this variation in such a way that the distal end of the slot (264) is slightly outside of the inflation area. That is, it extends beyond the end of the inflatable region of the balloon (242). Consequently, when the balloon is pressurized and expands by inflation with liquid supplied through the slot, it can be maintained in static size by continuous introduction of fluid from the proximal end, or should the fluid supply be stopped, the balloon will bleed down to a state of non-inflation as time goes on. This permits the fluid, even should it be a radio-opaque fluid used as a dye or other such viscous fluids, to progress without particular hesitation into the body region distal of the balloon. For some procedures, such a “bleed down” feature is desirable.

FIG. 4B shows a closable but open port (266) distally located in the depicted core guide member variation (268). The opening in port (266) is, again, sized to allow the fluid introduced into the guide member opening an opportunity to escape at a controlled rate depending upon the pressure build up in the core guide member and the viscosity of the fluid there.

Finally, FIG. 4C shows a variation of the device in which a closable port (270) is opened by removal of a plug (272) by proximal control. Removal of the plug (272) allows the end of the device to be opened so that fluid found within the guide member may then flow into the related body lumen. As will be discussed below, this concept may also be used to differentially inflate or deflate individual balloons in the multi-balloon structures shown here, with a single lumen structure.

Typical dimensions, provided only for guidance and not for purposes of limiting the scope of the variability and flexibility of the system in any way, for certain of the components are: the core guide member body inner diameter (ID) may be in the range of about 0.003-0.020 inches or perhaps 0.003-0.006 inches, the core guide member body wall thickness may be 0.0015-0.008 inches, uninflated balloon wall thickness may be 0.00075-0.00150 inches or 0.0004-0.002 inches, and a typical stenting device may have a wall thickness of 0.0015-0.005 inches. The choice of materials is a function of the use to which the component is placed. For instance, thinner materials may be used where smaller lumens are to be approached and passed, as may be found in the neurovasculature. More robust or thicker materials may be suitable for cardiovascular or genito-urinary service. Of course, the devices may be made of thicker materials, if so desired.

Balloon Catheter

FIGS. 5A, 5B, and 5C show a variation of the described system utilizing a balloon catheter in conjunction with one or more of a constraint member and a core guide member with associated balloon or a conventional guidewire.

FIG. 5A shows a balloon catheter (280) having one or more balloons (282) located in a substantially distal position on the catheter (280). Balloon (282) is shown both in its uninflated condition and, in dotted line form, in its fully inflated condition. The balloon or balloons used in this variation may be of the materials and function described elsewhere herein with respect to other balloons. Radio-opaque markers (284 and 286) as may be found in many balloon catheters are also shown in FIGS. 5A and 5B. These markers (284 and 286) are intended to show the position of the edge of the balloon distally and proximally as may be desired by the physician using this balloon. Many other marker systems, including a marker or markers placed within the length of the balloon or radio-opaque materials inherent to the balloon, are also acceptable. Although such a catheter (280) may be of the type typically used for treatment of atherosclerotic lesions or the like, as by angioplasty or stent placement, the catheter is limited only in that it generically must comprise at least one balloon of some type. Such balloon catheters specifically include those used in angioplasty procedures but also include any balloon catheter that is inflated or inflatable in the human body. Other balloon catheter structures, including multi-balloon structures, are described elsewhere herein.

Also shown in FIG. 5A is guidewire (288). Guidewire (288) may be a guidewire or cable capable of acting to guide the catheter (280) in some fashion, typically by manipulation from the proximal end, through passageways in existence in the body or those created in the body, perhaps for the passage of catheter (280). Guide catheter (290) is a catheter typical of those often used to allow passage of a medical treatment device in the intermediate region between the entry to the body and the region of the body to be treated. For instance, a guide catheter used for angioplasty of the arteries of the heart may extend between the entry point chosen—many times the choice is the femoral artery—up to a point in the vasculature just adjacent the heart, e.g., at a coronary ostium. Then, the smaller balloon catheter, often with the help of a guidewire, traverses the rest of the fairly short distance from the distal end of the guide catheter to the lesion in the arteries of the heart. Guide catheter (290) is depicted to include radio-opaque markers (292).

Also shown in this combination, is a constraint member (294). Constraint member (294) is described in more detail just below. The variation shown in FIGS. 5A and 5B includes one or more radio-opaque markers or regions (296). The radiographically visible members or bands are generally of such a combination that they permit the user to determine the position of constraint member (294) and the length of balloon (282) available for expansion and the amount already expanded. Constraint member (294) is to be controlled by the user of the balloon catheter system in such a way that, as is shown in FIG. 5B, it slides down the balloon catheter shaft and then over at least some portion of balloon (282) and by its presence restricts the radial inflation of balloon (282) over some longitudinal region. The constraint member (284) may be of a physical configuration and strength to be physically manipulated from the proximal end of the catheter system by the user.

FIG. 5C shows a balloon catheter (280) having one or more balloons (282) located in a substantially distal position on the balloon catheter (280) in conjunction with both a constraint member (294) and a core member (170) having a balloon region (182) and a distal radio-opaque coil (190). In this variation, the core guide member may be used as a guidewire to lead the larger balloon catheter (280) through the vasculature. The core member (170) and its attendant balloon region (182) may also be used to open or treat a lumen having a lesion where needed, prior to passage of the larger balloon catheter (280).

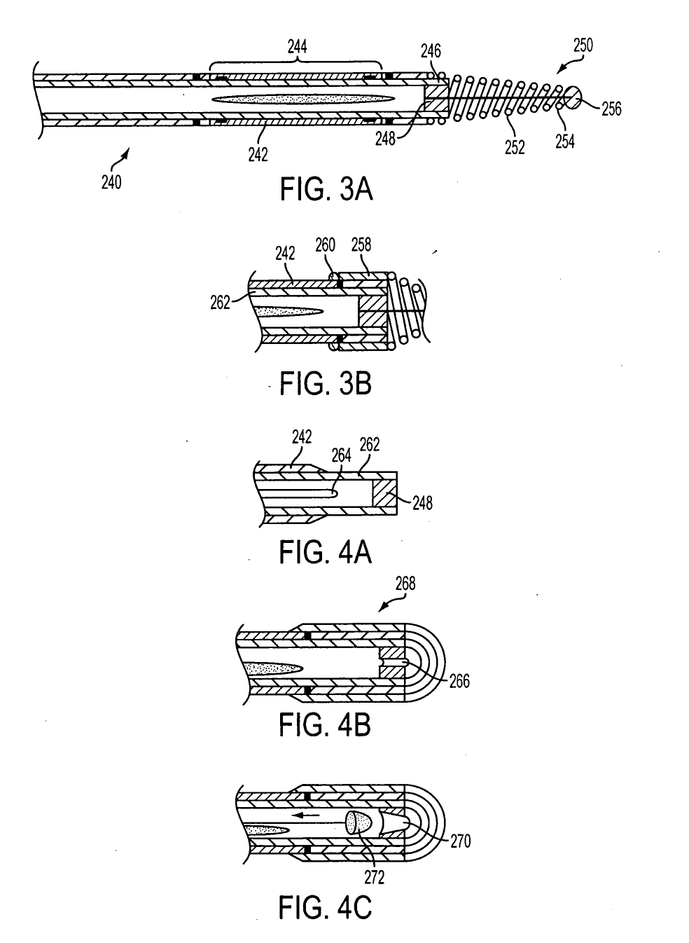

FIG. 6A shows a balloon catheter system wherein the balloon functions of the system shown in FIG. 5C are found on a single catheter (300). In this variation, the balloon catheter (300) includes a larger balloon (304) bracketed by optional radio-opaque bands (306, 308) and further includes a smaller balloon (302) located distally. This catheter is shown having a distal opening and having a guidewire (310) in place of the core guide member shown in the variation shown in FIG. 5C. The constraint member (294) is shown. This catheter may be used to first widen the lumen in a tight stenosis using the narrower distal balloon (302) and then using the larger balloon (304).

FIGS. 6B and 6C show a variation of the catheter and system found in FIG. 6A having separate inflation fluid supply paths to each of the distal balloon (302) and the proximal balloon (304). Also shown is the constraining member (294). The constraining member (294) is shown in a position where it partially constrains the expansion of the larger proximal balloon (304) thereby limiting its effective expanded length. The expansion of distal balloon (302) is independently controllable in this variation because of the independent inflation/deflation lumen.

FIG. 6D shows the FIG. 6A catheter variation having, in this instance, but one inflation lumen, i.e., a lumen (312) common both to distal balloon (302) and to larger proximal balloon (304). However, the constraining member (294) is shown holding the proximal balloon (304) in an un-inflated form even though the distal balloon (302) is expanded. This illustrates the cooperative and flexible usage of the constraining member, variously to control, to select, and to restrain the balloon in this variation of the system and on each of the other variations as well.

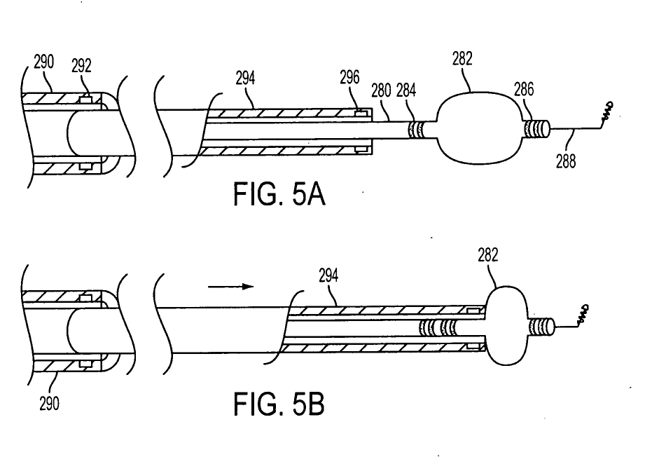

FIGS. 7A, 7B, and 7C show a number of expanded balloon shape variations suitable for use in this system, particularly with a constraining member. Specifically, FIG. 7A shows a balloon profile (320) with both a distal taper (322) and a proximal taper (324). FIG. 7B shows a balloon profile (326) that is stepped, having two different diameters (328, 330). FIG. 7C shows a balloon profile (332) that is stepped having a smaller distal diameter (334) and a larger proximal diameter (336) in combination with both a distal taper (338) and a proximal taper (340). These balloon forms or profiles and others having tapers and landings are useful in expanding stents, performing angioplasty procedures, and also for assuring the fit of stents to the vessel wall, i.e. “good “stent-vessel wall apposition,” and “touching up” the fit of stents in those vessels.

Constraining Member

As has been explained above, one feature of both the low profile balloon containing system using the core guide member and the system utilizing a balloon catheter is the ability of the system to control the longitudinal size of the balloon installed on the core guide member or the balloon catheter by sliding the constraining member on the outside of the deflated balloon (typically while the balloon is at least partially deflated) until a proper or desired balloon length is selected. Upon inflation of the respective balloons, the constraining member does not permit the balloon to expand at the balloon's proximal end. The balloon section beneath the constraining member remains uninflated; the balloon section outside of the confines of the constraining member, inflates. The distributed nature of the fluid flow pathway from the interior of the core guide lumen body or the catheter body is usually instrumental in allowing the balloon to be inflated.

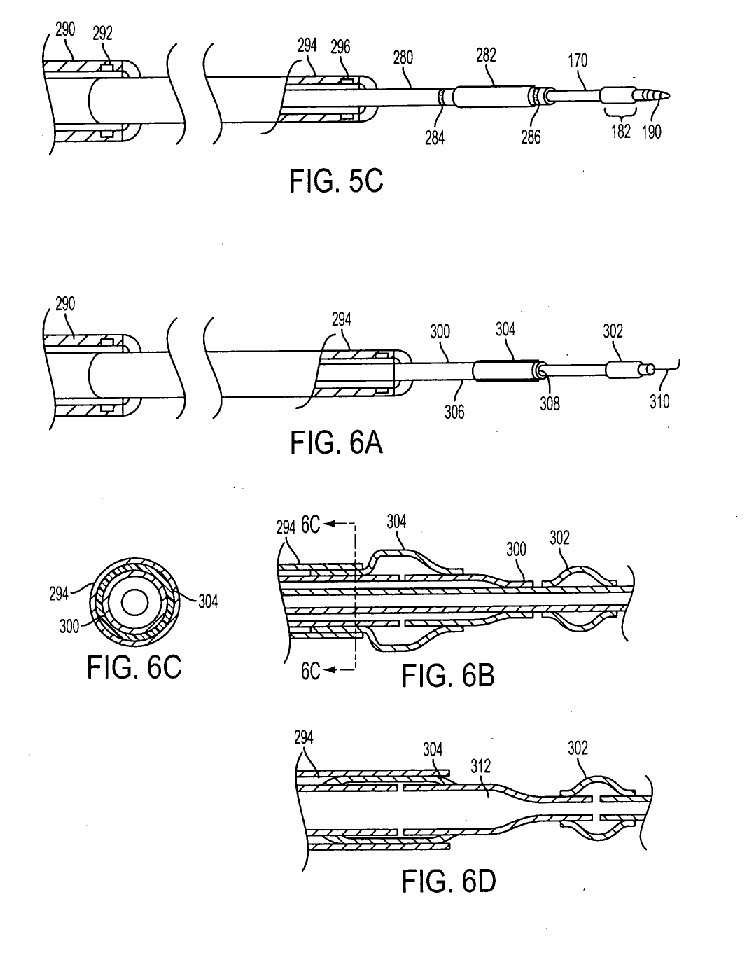

FIG. 8A shows the presence of a constraining member (350) in a sliding relationship to the core guide member (352). The balloon (354) is shown both in its uninflated form and in dotted “ghost” form (356) as inflated. The distal end (358) of the constraining member (350) is shown to be located distally of the proximal-most end (360) of balloon (354). The region of the balloon between the distal end (358) of the constraining member (350) and the proximal end (360) of balloon (354) simply remains uninflated.

FIG. 8B shows the same device as found in FIG. 8A. In this Figure, however, constraining member (350) has been slid distally a bit more and now constrains about two-thirds of the length of the uninflated balloon leaving but the inflated length of balloon (362) to perform whatever tasks the device is required to do. The diameter of the various inflated balloon shown in FIGS. 8A and 8B may be the same or different depending upon the nature of the balloons employed, e.g., non-compliant balloons generally have the same diameter no matter what length of constraining member is employed in limiting balloon expansion.

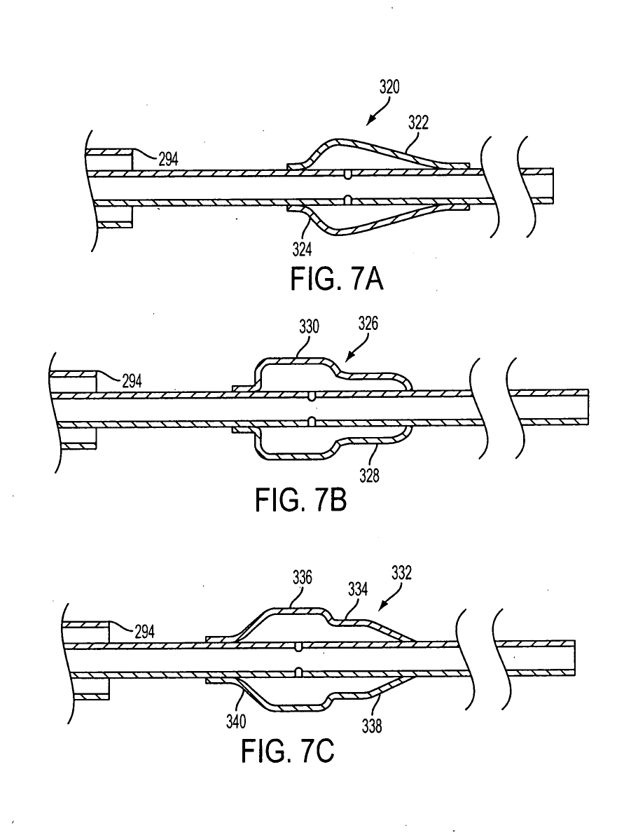

FIG. 9A shows a partial cross-section of a constraining member (370). This variation of the constraining member (370) is generally tubular and is sized so that it extends away from the outer wall of the core guide member or balloon catheter that is designed to fit inside. The constraining member may optionally include such additional subcomponents as the wiper or seal (372) interior to the distal end of the constraining member (370). An additional strengthening ring or radio-opaque marker (374) may also be situated at or near the distal end of constraining member (370). The wiper (372) may have a variety of functions. For instance, depending upon its axial length, the wiper (372) may simply displace an amount of fluid that would ordinarily be introduced to the interior of the balloon. It may serve to isolate the constraining member (370) from the surface of the core guide member found within to better allow the constraining member (370) to slide easily down the shaft of the core guide member. This spacing (376) is shown in FIG. 9B. Also shown in FIG. 9B is an ancillary radio-opaque member (378) found in the constraining member and a similar radio-opaque marker (380) found at the proximal end of balloon (382). The position of the various radio-opaque markers employed in a device such as this need only provide the physician-user with clear information about the length of balloon being constrained and its position in the human body. Markers such as those found at (378, 380) convey such information; other marker positions may convey such information as well.

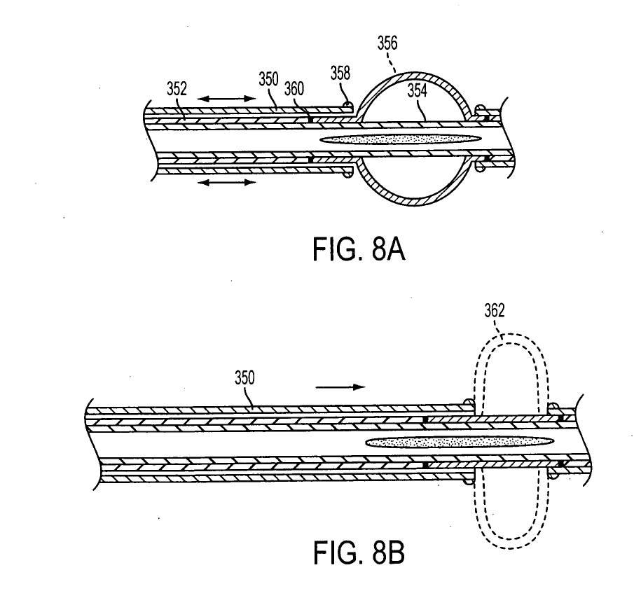

FIGS. 10A through 10D show another variation of constraining member (390). FIG. 10A shows a cross section of the distal end of constraining member (390). This variation is potentially lighter in that significant portions of the wall of the device have been removed or are not present, in any case. This variation would minimize the “stiction” that might exist between the core guide member or the balloon catheter and the interior of the constraining member (390). Additionally, as may be seen FIG. 10C, the radial spacing (392) between constraining member (390) and the underlying core guide member is significant. FIG. 10D shows the constraining action of constraining member (390) upon balloon (396) and the phenomenon that the balloon does inflate and contact constraining member (390) beneath the section covering the balloon. Care should be taken, though, to assure that any inflated balloon potentially extending through the openings (398) in constraining member (370) not be a hindrance to the procedure then being practiced.

Multiple Balloon Structures

Both the multiple balloon core guide member variation and the multiple balloon catheter variations discussed herein have the ability, when the proper configurations are selected, to allow the choice of stent size (both diameter and length as well as diameter or length) after the stent has been placed in the body. The system allows controllable expansion of the various expandable components (by inflation or otherwise) to expand one or more stents to a selected length or to a selected diameter, all without the necessity of removing the inflatable assembly from the patient's body. The combination of radially located balloons, longitudinally located balloons, and various sleeves, all as discussed herein, permit such facility of use.

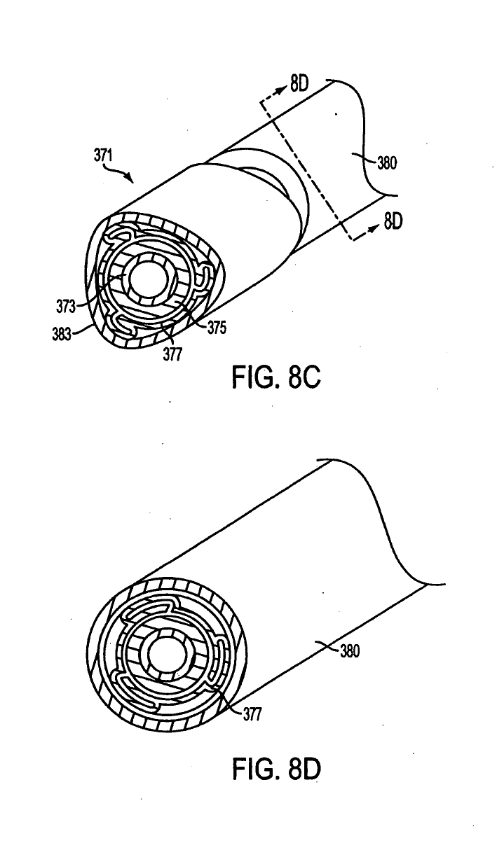

FIGS. 11A and 11B show a more complicated version of the variation of the described system involving a core guide member.

FIG. 11A shows a longitudinal, sectional view of that described low profile system and FIG. 11B shows a cross section of the system (400) at the noted site.

FIG. 11A shows an example of a multi-balloon device (400) in which the balloons are stacked, e.g., radially adjacent each other, and yet independently expandable. As may be seen more clearly in FIG. 11B, the exemplified core guide member body (402) includes three independent lumens or passageways (404). Except as noted below, each passageway has an opening into a separate balloon. In this example, opening (406) opens only into the inner-most balloon (412). Opening (408) is located in the body wall distally of the end (410) of the inner-most balloon (412) and because of that distal positioning is, by fluid communication, sited to expand only the second level balloon (414). Finally, opening (416) is situated past the distal end of middle-balloon (414) and proximally of the distal end of outer-most balloon (418) and consequently is able to inflate that outer-most balloon (418). A movable constraining member (422) is also shown in FIG. 11A. The constraining member (422) is used in the same way as the constraining members described elsewhere.

In addition to the multi-balloon stack section (424), FIG. 11A additionally shows a distal balloon section (426) extending distally past the region (428) of the balloon stack (424). The distal balloon section (426) may be an extension of any of the balloons found in the stack (424) or may be an independent component, but for this example, we have chosen to show it as an extension of outermost balloon (418) and proximally ended (or physically defined) with a cinch (436) surrounding outermost balloon (418). This cinch (436) allows the more distal portion (426) of the outermost balloon (perhaps in cooperation with the constraining member (422)) to be operationally independent of the operation of (inflation of) the stacked balloon section (424).

Again, for the example shown in FIG. 11A, opening (430) and the more-proximal opening (406) accept inflation fluid from the same passageway. An independent passageway may obviously be employed for distal balloon section, if the resources of the design are appropriate. Separate passageways may be desirable when different types of inflation fluids are to be used in different balloons, perhaps concurrently, etc. Designing for independent passageways may provide a benefit in preventing “drift” of fluid from one balloon to another, as well.

The variation shown in FIGS. 11A and 11B carries with it an ability to expand several balloons concurrently to attain several, perhaps different, diameters. By appropriate choice of either (or combinations of both) compliant or noncompliant balloons on the device, a wide variety of flexible configurations are achievable. Further, the balloons may be individually inflated for specific types of tasks. As an example, the inner-most balloon (412) may be inflated to provide a small diameter, expanded balloon section easily able to perform an angioplasty in a narrow region of vasculature. In essence, inflation of the inner-most balloon (412) alone provides a modestly sized balloon having a fairly thick wall. In some instances, the inner balloon desirably would be of a low compliance material, e.g., able to produce a specific diameter balloon. This also allows the inner-most balloon to effectively accept higher pressure inflation fluids than would a balloon comprised of an elastic, elastomeric, or compliant polymer. In the event that the more inner balloon or balloons are of a generally inelastic material, the outer-most balloon (418) may be made of an elastic or semi-elastic material in order to help assure that the inner nonelastic balloons return to their original shape for later retrieval of the device.

If a larger balloon size is needed, the middle balloon may (414) may be inflated, with or without the concurrent inflation of the inner-most balloon (412). But, inflation of the middle balloon (414) is more rapidly achieved by concurrently directing inflation fluids both to the inner-most balloon (412) and to the middle balloon (414) using slots (406, 408) in parallel. Similarly, when deflation of the balloons is needed, e.g., for removal of the catheter, the ability to use both passageways for inflation fluid exit is valuable, particularly as to the speed of inflation.

Further, in a situation where the user determines that the inner-most balloon (412) provides too small a profile, it is a simple matter to maintain the inner-most balloon (412) in an inflated state perhaps to hold a stent in position or to hold the core guide member in place and to inflate the middle balloon (414) to gather a larger profile. An excellent way to maintain the balloon in an inflated state is to retain the fluid in the balloon, but other methods of holding the inflation are acceptable.

This procedure of holding the fluid in an inner balloon while filling an outer balloon may be practiced on any of the combinations of balloon shown here. Additionally, in some variations, one or more inner balloons may be filled while holding the fluid in an outer balloon or balloons. This may be achieved, for instance, by using check valves or one-way stop valves, as will be explained later in more detail. Other devices and methods useful in holding the fluid in the balloon are also available.

Returning to FIG. 11A, since the distal balloon section (426) shown there may have a profile that is significantly smaller than the stacked balloon section (424), the distal section (426) may be useful in opening and dilating narrow lumenal passageways for later (or concurrent) stenting or for simply providing a larger passageway such as may be needed for passage or use of the balloons in the stacked balloon section (424).

In the variation of the device having multiple stacked balloons shown in FIGS. 11A and 11B, the lumen serving inflation fluid to the inner-most balloon (412) also provides inflation fluid to the distal section (426) via an opening (430). This design permits, amongst others: a.) inflation of both the inner-most balloon (412) and the distal balloon (440) found in the distal balloon section (426) together using but a single lumen (418) (by passage of inflation fluid through opening (406) into inner-most balloon (412) and by passage of inflation fluid through opening (430) into distal balloon (440)), or b.) by manipulation of the constraining member (422) to constrain inflation of the balloon stack section (424), inflation of the distal balloon (440) of distal balloon section (426) alone. Again, this shows the substantial operational flexibility of the system.

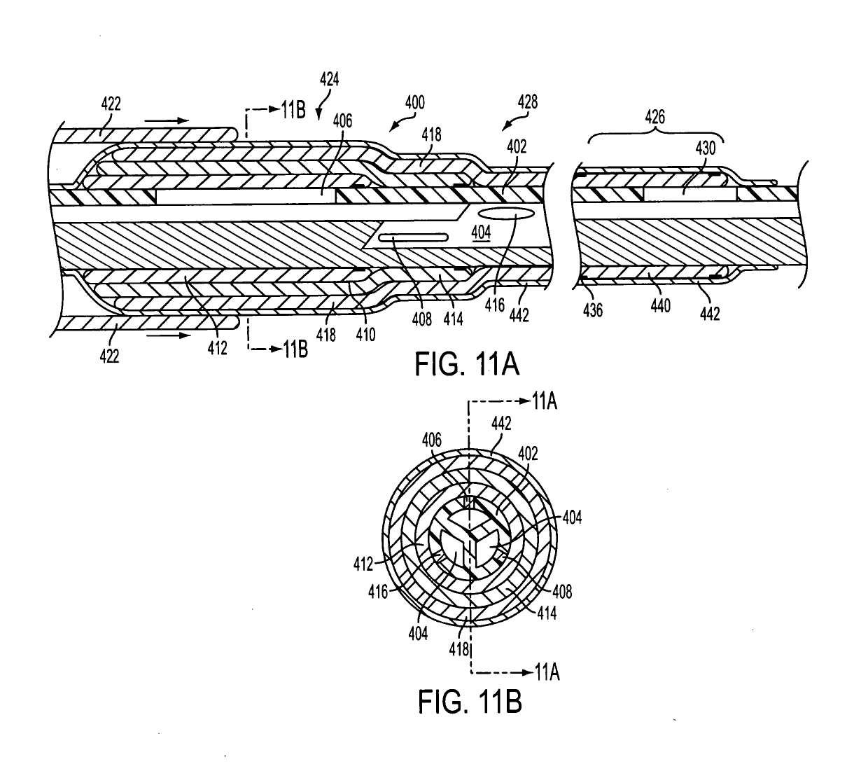

An optional, elastic sleeve (442) is shown on the outer surface or outer side of the balloon stack (424) in FIGS. 11A and 11B and is used to help remedy the need to collapse the balloons into a small diameter after deflation; this version of the elastic sleeve (442), as shown here, is not independently inflatable. In other variations, it may be inflatable. In any event, the depicted layer would be for the specific purpose of returning the balloons to (or towards) their pre-expansion size. The elastic sleeve (442) is shown extending onto the distal balloon section (426) for the same purpose. The elastic sleeve (442) may be optionally (and independently) placed either on the distal balloon section (426) or on the balloon stack (424) or on both. The assistance that the elastic sleeve (442) provides in returning the various balloons to their original size is typically more pronounced with regard to the distal balloon section (426) than to the balloon stack (424) because (as discussed below with respect to FIGS. 13A and 13B) the constraining member (422) may be used to re-form the stacked balloon section, but that constraining member (422) is usually too large (in interior diameter) to similarly re-mold the narrower distal balloon section (426). Use of the elastic sleeve (442) also remedies the potential problem of “stiction” between balloons that often occurs even in single balloons when the inflation fluid is removed by vacuum and the suction blocks the exit port by pulling the balloon wall against the exit port or by causing lumen collapse. That is to say: the step of withdrawing inflation fluid from the balloons in typical balloon catheters is quite difficult, particularly in smaller and in multiple balloons, because the fluid is withdrawn using a vacuum source, often a simple syringe. The flexibility of the various polymeric components creates a situation in which the catheter walls may move with the vacuum and seal or clog the exit ports or slits. Additionally, that flexibility causes the catheter flow lumen to narrow also tending to slow fluid flow. The presence of the elastic sleeve (442) is particularly important at the end of the deflation cycle, in that it provides an impetus, a specific added positive pressure, pushing against the fluid-filled balloon or balloons, causing the inflation fluid to flow outward towards the proximal end (toward the inflator/deflator) under an elevated pressure. That pressure is situated at the balloon. In most systems used today, the fluid is withdrawn under a vacuum, the source of which is outside the patient's body. Fluid removal rates are facilitated. Additionally, the elastic sleeve (442) may be situated to be interior to the non-compliant balloon, perhaps by causing it to adhere to the interior of the balloon or by co-extruding it with the balloon. This elastic sleeve (442) has the potential, when properly sized, to significantly enhancing the deflation times.

Not shown in FIGS. 11A or 11B are the generally suitable radio-opaque regions or markers. As has been noted elsewhere, such markers may be placed on the various balloons and upon the constraining member to permit the user to visualize the positions of the various components as needed.

The constraining member (422) may be used to push (or to re-form) the shape of the deflated balloon pack to a smaller profile and a vacuum may be pulled on the various fluid passageways to extricate the fluid and to pull down the balloons to size. In overall effect, the constraining member (422) may be used as a mandrel to press the deflated balloons into a size similar to the inner diameter of the constraining member (422). Such an urging may perhaps be with the assistance of the elastic sleeve (442) and any pre-forming or “memory” found in the balloons themselves. Non-compliant balloons are typically folded in some fashion as initially used, and have a regular, low profile. The balloons often have three, four, or more “wings” that are folded flat when the balloon is initially produced; but, once inflated, those wings may be difficult to re-position. The constraining member (422), elastic sleeve (442), balloon member “memory,” and any lubricant added between balloons to allow inter-balloon slippage tend to cooperate in shrinking deflated balloons to a smaller diameter even if the diameter isn't the small value found before inflation. Moreover, these components, particularly constraining member (422) and elastic sleeve (442), enhance liquid removal during the deflation stage due to the positive pressure they apply to the liquid.

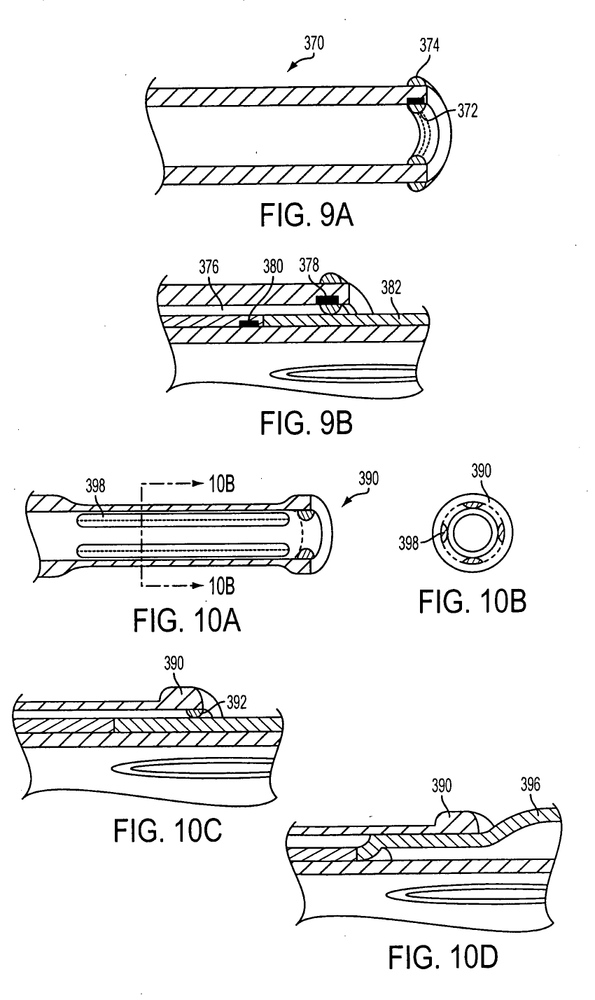

FIG. 12A shows a partial longitudinal section of a balloon (450) having a radial stack of three balloons: an innermost balloon (452), a middle balloon (454) and an outer balloon (456). These balloons (452, 454, 456) are filled using components or passageways not otherwise shown in this Figure. Each of these balloons is independently inflatable by the physician-user. In this example, each of the balloons is adherent to a central tubular member (458) having an open lumen (460) suitable for placement of a guide wire. Of particular note in this catheter section (450) is the placement of the valve wire (462). This valve wire (462) passes from lumen (460) through the wall of central tubing member (458) and passes beneath the regions of each of balloons (452, 454, 456) in such a way that it blocks in each balloon, a deflation passageway (468) as may be more clearly seen in FIGS. 12B and 12C. FIGS. 12B and 12C show a cross-sectional view of one end of the catheter section (450) showing the positioning of wire (462) and the way that it blocks or fills deflation passageway (468). FIG. 12C is a close-up of the circled cross-sectional area (480) shown in FIG. 12B.

The valve wire (462) may be used in the following fashion. Once the balloons are all inflated as shown in FIG. 12A, the simple removal of the valve wire (462) will result in open passageways (468) from the interior of each of the balloons (ultimately) into both of the interior lumen (460) and to the space surrounding the catheter section (450). This, obviously, allows immediate deflation of each of the balloons. The catheter section (450) is, practically speaking, no longer inflatable.

Valve wire (462) may be used as a valve to differentiate amongst the three balloons. That is to say, by moving the valve wire (462) to a position wherein the end of the valve wire is in the space between outer balloon (456) and middle balloon (454), the middle balloon (454) and the inner balloon (452) remain inflatable, but the outer balloon is not. This may be of value when a simpler fluid feed system is used to inflate the various balloons, e.g., a single lumen inflating the multiple balloon stack. Thus the simple movement of pulling valve wire (462) pre-determines the final size of the deployed stent. In this case, pulling the wire prior to inflation will determine which balloons are to be inflated and the resulting stent size.

FIG. 13A shows, for the sake of ease of illustration, a cross-section of a simple stacked multi-balloon structure (480)—“simple” in the sense that it has but two balloons rather than the multiple balloons in the example above. In this illustration, may be found a core guide member (482), an elastic or compliant balloon (484), an inelastic or non-compliant balloon (486), and an outer elastic sleeve (488) as shown in FIG. 11A, and a constraining member (490). The two balloons in this example have been previously inflated and are now being deflated and the profile is now being adjusted back to a lower, more narrow profile. As may be seen in FIG. 13A, the inner elastic balloon (484) has deflated to a regular form. The outer inelastic balloon (486) has been partially re-formed by the outer elastic sleeve (488) in that the “wings” on that balloon (486) have been partially folded. FIG. 13B shows the further re-formation of the outer balloon (486) by sliding the constraining member (490) across the stacked multi-balloon structure (480). As may be apparent, it is typically desirable to return a previously folded non-compliant balloon to its original conformation since that would be a smaller diameter. However as is shown in FIG. 13B, such a precise re-folding is not always possible. This system has the ability to minimize the diameter even when the folding is not perfect.

In addition, folding the balloons using a predictable configuration, such as a spiral formation is useful, both in assessing the final diameter of the inflated balloon (and its colleagues in the balloon stack) and in helping to predictably re-fold the balloons when deflated and used in a stack. Use of lubricants upon the outer surface of the non-compliant balloons, to lessen the friction against the next-outer balloon, also helps remedy the return.

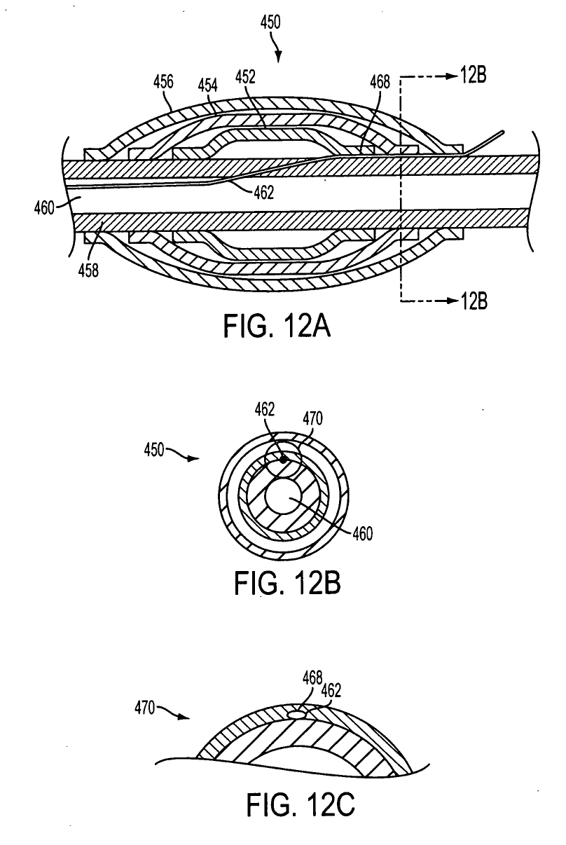

FIGS. 14A and 14B show two variations of an assembled guide member having a number of lumens that may be used in a multi-balloon device such as described just above. The guide core body (500) in FIG. 14A is made up of a number of conduits with central passageways of suitable size, perhaps small (502), perhaps larger (504). The variation shown in FIG. 14B shows a number of conduits (508) with openings (510) through which balloons may be inflated. The various tubing members may be round, as shown, or any other appropriate form having a passageway for fluid passage. The resulting core guide member cross-sections may be symmetric such as shown in FIGS. 14A and 14B, but need not be. The various conduits (502, 504, 508) may be polymeric tubing and may be of one or more diameters and may be mixed with other formats (solid wire, ribbon, braid tube, coil tube, etc.) as desired.

FIGS. 14C and 14D show cross-sectional and side-cross sectional views of a further variation of a multi-passageway core guide member (512) having multiple interior tubes (514, 516, 517, 518) each with a passageway. FIGS. 14C and 14D show the termination of the tubes (517, 518) into orifices (520, 524) for inflation of stacked balloons (not shown). The interior tubes (514, 516, 517, 518) may be polymeric, e.g., the Nylons such as Nylon 12. If the material chosen for the interior tubes allows them to collapse, each of the included tubes may be used in such a way that they themselves inflate and, in doing so, press or squeeze the others to a small residual space within the outer member (522). Thus, each of the included tubes (514, 516, 517, 518) may be used as if it were a tube having a much larger inner diameter. One may also achieve such an effect with concentric tubing, as well.

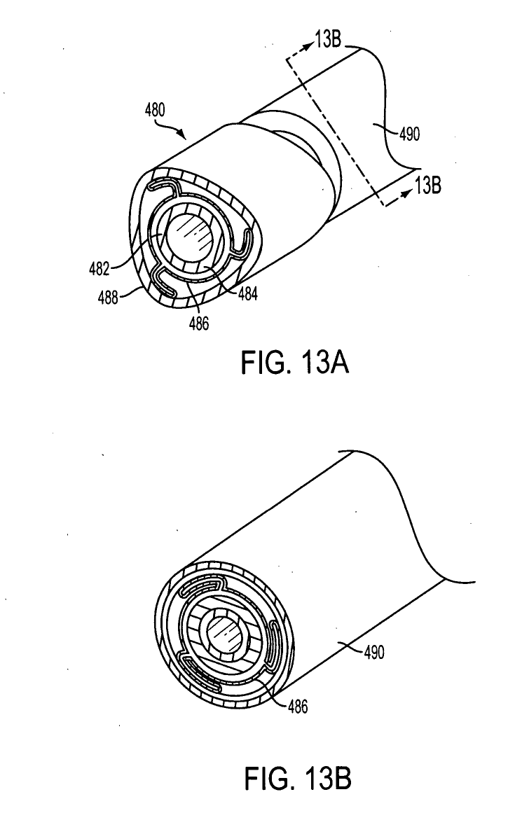

FIGS. 15A, 15B, 15C, and 15D show the use of concentric tubes to provide inflation fluid to the stacked balloons shown there. These tubes may be used in conjunction or combination with the small tubes and divided annular passageways discussed elsewhere herein to pass inflation fluid to multiples of balloons. FIGS. 15A and 15B show a stacked balloon catheter (530) having an open central passageway (532) opening to a small balloon (534). The middle balloon (536) is attached to a middle tubing member (538). The middle tubing member (538) forms an annular space and passageway (540) that fills balloon (536). The outer tubing member (542) forms an annular passageway (544) that fills outer balloon (537).

The concentric tubing designs shown in FIGS. 15A and 15B may suitably use a variety of materials for the various tubing members. For instance, the central-tubing member surrounding lumen (532) may comprise a comparatively stiffer polymer, e.g., Nylons, poly(aryl ether ether ketone) (PEEK), polyether sulfones, polyimides, polyoxymethylene (POM—Delrin), PEEK and its analogous siblings (PEKK), thereby allowing smaller thin wall tubing, maintaining catheter pushability, and maintaining the central inflation (and deflation) lumen in an open condition.

As an alternative, the outermost tubing may be selected as the more stiff, or thicker, of the group to promote pushability or torqueability and lower twisting hysteresis effects. In such a variation, the inner tubing members may be chosen of materials that are collapsible, e.g., silicones, various of the Nylons, polyethylene, perhaps mixed with PVA, and the like, thereby allowing one or more of the other tubing members to act as if they were of a much larger diameter. Since the selected tubing member then contains pressurized liquid within, that tubing member may expand or unfold (if it had either been collapsed or if it comprises an elastomeric material) and press upon the other non-selected lumen thereby compressing them.

The concentric tubing catheter variation (530) shown in FIGS. 15A and 15B may be stiff in the region of the balloons. To improve the flexibility of that version, or other balloon catheters disclosed here, the concepts exemplified in FIG. 1SC may be employed. Bellows (550, 552, and 554) may be introduced into the various tubing members to enhance flexibility at or near to, the balloons. Bellows (550, 552, and 554) shown are shown variously as located within a balloon member (552, 554) and exterior to the balloons (550). They are shown to be located adjacent to but proximal of the respective balloons (bellows (550) proximal of balloon (537) and bellows (552) proximal of balloon (536)) and to be located adjacent to, interior of, and distal of the balloon joint (bellows 554 is within lumen (532)).

FIG. 15D shows a variation in which the bellows (560, 562, and 564) are all longer than those shown in FIG. 15C, proximal of their respective balloons, and able to provide significantly greater catheter flexibility in that region of the catheter.

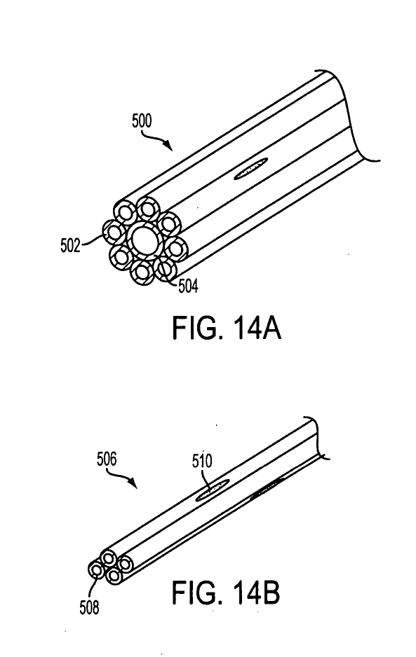

FIGS. 16A, 16C, and 16F show cross-sections of a number of configurations suitable for controllably providing fluids to the balloons of the multi-balloon catheters discussed here. FIGS. 16B, 16D, and 16F show longitudinal cross-sections of those configurations.

FIG. 16A shows a cross-sectional view and FIG. 16B shows a longitudinal cross-section of a catheter body (570) having a central passageway (572) typically used for a guidewire. The catheter body (570) also has a number of passageways or lumens (574) that may be selected for introduction of fluid into a more distal balloon. As an illustration of the delivery of such a fluid, one lumen (574) has been plugged (576) and an opening (578) provided in the outside wall. In this instance, a balloon mounted on the outside wall and surrounding opening (578) could be inflated using the corresponding passageway.

FIG. 16C shows a cross-sectional view and FIG. 16D shows longitudinal cross-section of an assembled catheter body (580) having a central passageway (582) typically used for a guidewire. In this variation, the center tubing (584) surrounding lumen (582) would typically be used as a structural backbone to the catheter body (580). The surrounding tubing components (586) may be used, as needed, for delivery of inflation fluids to balloons. This variation includes one supply end (588) extending through the heat-shrink polymer covering (590). Again, placement of a balloon over the supply end (588) allows the corresponding supply tubing (586) to be used for inflation fluid supply and deflation. The remaining tubing components (586) similarly may be extended through covering (590) to provide a pathway for inflation fluids to pass to the exterior balloons.

FIG. 16E shows a cross-sectional view and FIG. 16F shows longitudinal cross-section of a catheter body (590) having a central passageway (592) typically used for a guidewire. In this variation, the center tubing (594) may be less structural than is the one shown just above. The stiffer member, as a matter of choice by the catheter designer is the outer tubing (596). As was the case with a variation mentioned above, if the various fluid tubing members (598) are chosen in such away, e.g., by choice of tubing size or material, that they can collapse when other selected tubing members are filled with pressurized fluids, those tubing members actually being used for transport of fluid to a balloon will push the other members out of the way and compress them into a background role.

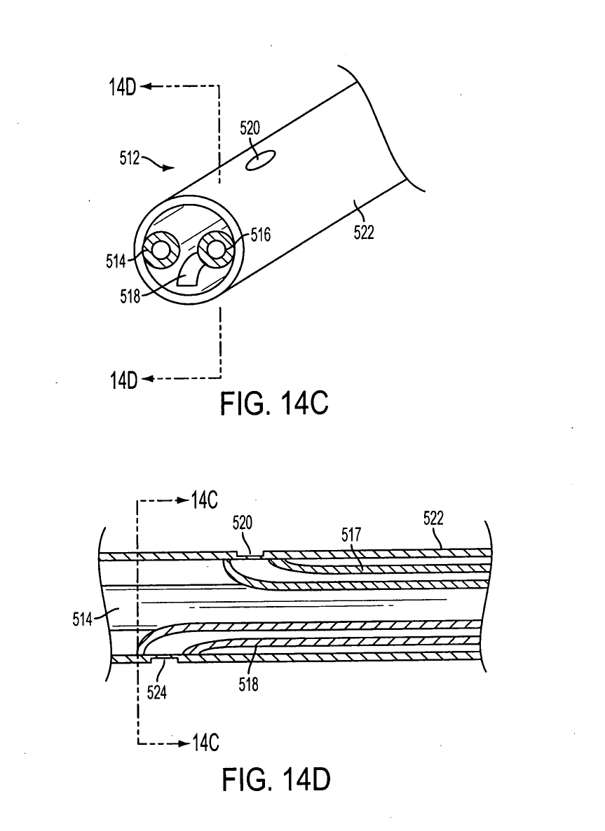

FIG. 17A shows a longitudinal section view and FIGS. 17B, 17C, and 17D provide cross-sectional views of a catheter body (600) having a central passageway (602) typically used for a guidewire. This section (600) is especially useful in the vicinity of the balloons, those that are distal in the design. In particular, portions of two balloons, an outer balloon (604) and an inner balloon (606), are shown in-the Figures. Each of them adheres-to the underlying central tubing member (608). In this variation, the inflation fluid is supplied to the balloons via small tubing supply members—small supply member (610) supplies outer balloon (604) and small supply member (612) supplies inner balloon (606). As may be seen in FIG. 17B, the small tubing member (612) passes beneath the edge or end (614) of the outer balloon (604). Using a sacrificial heat shrink tubing to make the balloon distal end (614) adhere to the underlying tubing member (608) may be desirable. Similarly, balloon (606) adheres to the tubing (612). Maintaining the patency of the small tubing member (612) during such construction may require the use of a small mandrel, e.g., a wire of an appropriate size, placed in the lumen of the small tubing member (612). Some additional filler (616) may be needed as shown in FIG. 17B. The process of heat-shrinking using a sacrificial heat shrink tubing will often produce the result shown in FIG. 17C where no additional adhesive or filler is needed. It should also be understood that by alternate choices of construction, the overall boundaries between the original components will facially disappear. For instance, by choice of materials having very similar properties or having significant miscibilities, as shown in FIG. 17D, the structure loses the otherwise distinct boundaries found in the cross-section found in FIG. 17B.

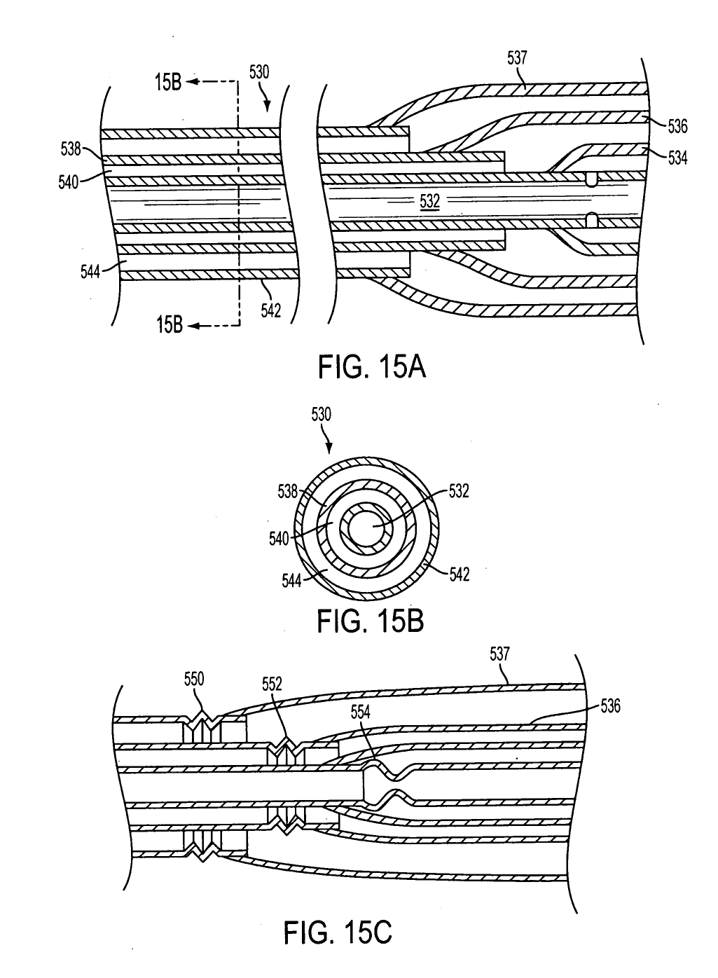

FIGS. 18A and 18B show an additional assisted folding balloon structure (620) of the type discussed above. This structure (620) includes a non-compliant balloon (622) folded as manufactured and includes an exterior elastic restoring sleeve (624). FIG. 18B is perspective view of the balloon shown in FIG. 18A. As noted in FIG. 18B, FIG. 18A is a cross sectional view of the folded balloon with the elastic retaining sleeve maintaining the folded shape. We have found that the retaining sleeve (624) will greatly assist the inner non compliant balloon return to its initial shape after inflation and deflation. The outer retaining sleeve (624) if chosen with an appropriately thin wall thickness, does not substantially interfere with the operation of the balloon (622) during dilation or otherwise.

FIGS. 18C through 18F show the use of a distally slidable balloon retaining member (626) to re-approach the initial diameter of a winged non compliant balloon (622).

FIG. 18C shows the balloon (622) fully inflated. FIG. 18D shows the balloon (517) with a number of wings (628) ready for collapsing. Because balloon (622) is non compliant, and is made of material such as Nylon or the like, the balloons typically are folded in some fashion to allow a lower profile.

FIG. 18E shows the sleeve (626) sliding onto balloon (622) and collapsing wings (628) into its interior. Finally, FIG. 18F shows the completion of the task of sleeve (626) in assimilating various extensions of the balloon into its interior to achieve a significantly lower profile.

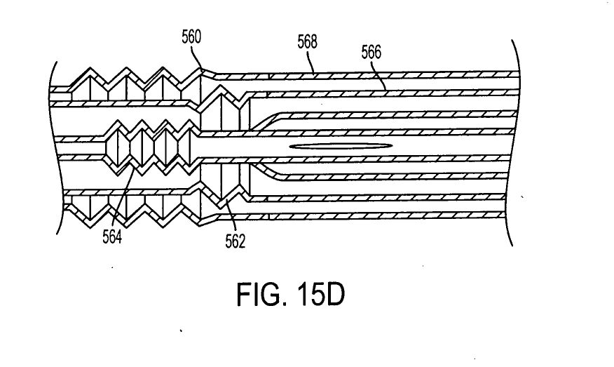

FIGS. 19A, 19B, and 19C show another variation of the described system including a balloon catheter of a conceptual design having a particular utility in this system. This variation also illustrates the concept that multiple balloons may be placed on a balloon catheter and those balloons spaced axially or radially with respect to each other, as the need arises. In any case, balloon catheter (630) includes a number of independently inflatable balloons: outer balloon (632) and inner balloon (634) positioned more proximally and low profile balloon (640) positioned more distally. Although only two more-proximal balloons are shown in this variation, as is discussed in the referenced application, many more variations of stacked balloons and axially spaced balloons may be utilized for a variety of medical purposes. As is shown in FIGS. 19B and 19C, the interior of the catheter body (636) is divided into four separate lumens. Three of the four lumens shown in this variation are provided for independent fluid flow to (and from) fluid source to allow independent inflation the various balloons of the catheter (630) as needed. In this variation, one passageway is utilized for the passage of a guidewire (638). The core guide member described elsewhere may also be utilized as a guide member in this combination. FIG. 19B shows the balloons (632, 634) to be independently inflatable by the use of those lumens. Again, it is not critical that the various lumen shown in this variation be used as specifically described, this description is provided simply to show the concept of a single lumen providing a single fluid to a single balloon. Other variations are as acceptable for the balloon catheter combination described herein. Indeed the various core guide member lumen structures discussed above with regard to FIGS. 11A, 11B, 12A-12C, 13A, 13B, and 14A-14D are also suitable for these balloon catheter structures as well.

FIG. 19C shows a cross section of the balloon catheter (630) at a more distal location. In that location, a single layer of a low profile balloon (640) is shown. As discussed above with relation to the multi-balloon core guide member above, a balloon catheter having a configuration such as this is especially useful in some tighter vascular lesions. For instance, the distal balloon (640) may be first expanded in a tighter lesions to provide a first level of angioplasty treatment thereby allowing distal movement of the balloon catheter in the blood vessel, followed by one or more expansions of the more proximal balloons (632, 634) as appropriate.

Additionally, the placement of an elastic covering (such as 442 in FIGS. 11A and 11B) is often useful in the design shown in FIG. 19C to maintain the overall low profile of the catheter by collapsing the larger balloon either with or without the cooperation of a suction source. Such an elastic covering is also particularly useful in collapsing balloons, whether found in a multi-balloon core guide member or a multi-balloon balloon catheter, when the device is used in a procedure having multiple inflations and deflations of those balloons.

The balloons in each of these variations of the system, as will be noted below in more detail, may be compliant, semi-compliant, or non-compliant and comprise elastic, elastomeric, semi-elastic, or non-elastic materials or combinations of them, variously admixed or layered as needed for a specific design.

Furthermore, as will be discussed below in more detail, the multi-layer balloon catheters shown variously in FIGS. 11A, 11B, 15A, 15B, and 19A may be used to provide a variety of specific and variable inflated balloon diameters and, when used to expand stent structures, similarly provide coordinate, dilated stent diameters. The balloons may be used in conjunction with the constraint members discussed elsewhere—see, for instance, 422 in FIG. 11A and 294 in FIGS. 5A and 5B—or the differential filling of the balloons to adjust the effective length of the inflated balloons, with or without adjusting the diameter, as will be discussed below. The stents to be delivered may be mounted either on the exterior of the multi-layer balloon catheters discussed herein or may be placed upon the stent sleeves also described elsewhere herein.

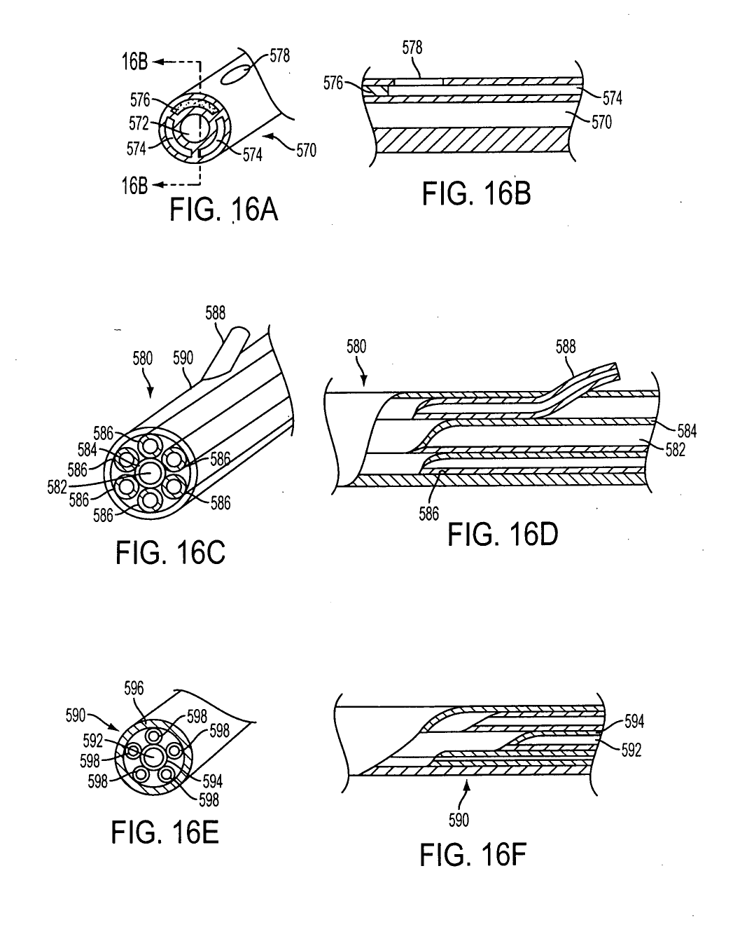

Examples of balloon configurations permitting changes in expanded length are found in FIGS. 20A, 20B, 20C, and 20D. In these drawings, the various independent inflation fluid feedlines are omitted to allow clarity of explanation of the balloon configuration operation.

FIGS. 20A and 20B are similar excepting that the balloons in FIG. 20A are each of the same diameter and in FIG. 20B, they are not. FIG. 20A shows a multi-balloon, stacked balloon catheter (650) having a central core tubing (652) with three attached balloons: an inner balloon (654), a middle balloon (656), and an outer balloon (658). The three balloons in this example are non-compliant and each has substantially the same diameter. They are shown to be inflated. Since the thickness of the various balloon materials is in the region of a few microns, the difference in thickness is not particularly significant. Consequently, the overall diameter of the balloon stack in the multi-balloon region (660) is not significantly different whether the outer balloons are sized to compensate for the balloons beneath or not. In a practical sense, then, for the placement of stents or for an angioplasty procedure, the diameter of the balloon stack shown in FIG. 20A is the same whether the one or all of the balloons are inflated. Only the functional length changes.

The catheter (662) shown in FIG. 20B is similar in form to that both seen in FIG. 11A and in FIG. 20A. In this example, the outer balloon (664), the middle balloon (666), and the inner balloon (668) each have different inflated diameters. Consequently, both the diameter and the effective length of the inflated balloon region change as the various balloons are inflated and deflated. In FIG. 20B, the three balloons (664, 666, and 668) are shown to be inflated. There is an apparent space between the inner diameters of the more outer balloons and the outer diameters of the next inner balloon in the stack.

The balloon catheter section (670) seen in FIG. 20C has a number of relatively “squarish”-cross-section balloons (672, 674, and 676). As those balloons are independently inflated, the effective length (678) of the inflated balloon changes, but the diameter does not.

The balloon catheter section (680) seen in FIG. 20D also has a number of relatively square cornered-cross-section balloons. In this variation, however, there are both inner balloons (682) and outer balloons (684). As the inner and outer balloons are independently inflated, the effective diameter of the inflated balloon is changed. When adjacent inner balloons are inflated or adjacent outer balloons are inflated, both diameter and length are changed. In any case, the control of either change is in the hands of the user. This may be achieved by differentially inflating outer balloons (684) and inner balloons (682) and effectively provide a low compliance balloon with several distinct diameters and lengths and hence, if a stent is to be used with catheter section (680), that singular stent may be expanded to different chosen sizes and lengths “on the fly.”

FIGS. 21A, 21B, 21C, and 21D show side view sections of balloon catheters having stents mounted on the balloons and that include manually placed exterior sheaths situated to prevent expansion of some section or portion of the mounted stents.

In some instances, when using the systems described here, it may be the case that more than one stent is mounted upon an expandable balloon that is a portion of the balloon catheter. Although we have explained many ways to implant some portion of stents mounted on the balloons of the balloon catheters, there may be instances in which a simple manual placement of a sheath upon some portion of a stent array is desirable since it allows the user-physician to implant the remaining stents at the selected site, but retain others on the balloon catheter, perhaps for the purpose of either using the remaining portion in a subsequent procedure, or set the length of the stent to be deployed.

FIG. 21A shows a section of a balloon catheter (690) having a stack of expandable balloons of a type described elsewhere herein. Exterior to the balloon stack (692) is a set of three stents (694, 696, 698). In a normal operation of our system involving such an array of stents, were it the case that less than all of the stents (694, 696, 698) were to be deployed, a movable constraining member or movable shape forming member would be placed over the stents not to be deployed. In this instance, a manually positioned exterior sheath (700) has been placed by the user-physician exterior to the two more-distal stents (696, 698). This sheath will prevent the balloon from expanding beneath those two stents while allowing the balloon or balloons in the balloon stack (692) to expand and deploy stent (694).

FIG. 21B shows the placement of stent (694) by inflation of the two inner balloons in balloon stack (692). The manually applied sheath (700) has done its task and prevented the underlying balloons from expanding and implanting stents (696 and 698).

Similarly, FIG. 21C shows a more distal section of a single layer balloon catheter (702). Balloon catheter (702) is made up of a single layer of balloon (704) and includes four mounted stents: two of which are situated for implantation (706) and two of which (708) are situated beneath manually applied exterior sheath (710).

FIG. 21D shows the expansion of balloon (704) resulting in the implantation of stents (706). The sheath (710) has maintained the balloon in position and retained stents (708) upon the balloon (704).

This means that instead of manufacturing various length balloons and stents, using the described system, one may instead produce but a single catheter model with detachable stents and then set the length of the stent to be deployed in the catheter lab by attaching the exterior sheath.

Many of the commercially available stents have a significant expansion tolerance, that is to say that the ratio of largest attainable expanded stent diameter to the delivered diameter is quite large and the stents are suitable for supporting lumens anywhere in that diameter range. The multiple layer balloon designs noted herein are suitable for selection of a desired stent diameter and of the length “on the fly” and attainment of those dimensions by selecting the balloon(s) to be inflated for a particular chosen stent size. With these designs, it should not be necessary to remove the catheter or core guide member from the patient's body while selecting the best treatment for the patient.

Deflation Aids

Mentioned periodically throughout this disclosure is the concept of incorporating into or situating adjacent to an inflatable member, a deflation aid having the specific function of aiding in the deflation of one or more inflatable members by providing a residual pressure in that inflatable member tending to “squeeze” the inflation fluid out of the volume (perhaps by compressing the inflation fluid in the volume), after the inflation pressure has been released. Discussed at greater length just below, are a variety of deflation aids in which the functionally operable component comprises an elastic member or component that is expanded during the step of inflating the inflatable member. This expansion step stretches the elastic material of the deflation aid and stores potential energy there by placing the elastic material in tension during the period the inflatable member is at least partially inflated, and stores potential energy there. Such tension is manifested as a significant pressure, referred to just above as a “residual pressure,” and will tend to push the fluid from the inflatable member volume and to collapse the object inflatable member or members. In many of the variations disclosed here, since the deflation aid is situated such that it accumulates energy during inflation of the inflatable member, during such deflation, in addition to pushing fluid from the inflatable member the deflation aid may also physically return that inflatable member (or at least “tend to” return that inflatable member) to its pre-inflation shape, perhaps by re-folding the inflatable member. In most of the variations discussed in this section, the deflation aid is not inflatable independently of the inflatable members it aids.

In contrast to the elastic deflation aids noted here, we have discussed elsewhere in this description, the use of constraining sleeves in our systems. These constraining sleeves may be moved with respect to the expandable members, e.g., inflatable balloons, for a variety of reasons and used to prevent the inflation of at least some portion of a specific balloon in a particular procedure, e.g., to expand only a portion of a stent to a particular diameter. In any case, in additional to the concept of preventing balloons from inflating, the sleeves may be used to aid in the deflation of one or more balloons (variously located radially adjacent to each other, longitudinally or axially adjacent each other, or combinations of the two) by moving the sleeve onto the balloon or balloons upon deflation to provide an elevated pressure in the inflation fluid and to therefore provide an impetus on the fluid and cause it to flow proximally towards the user out of the body. The constraining sleeves may be used independently to aid in deflation or in conjunction with the elastic deflation aids.

One variation of the elastic deflation aid is that shown as elastic sleeve (442) in FIGS. 11A, 11B, and 11C. That variation provides an elastic layer that is exterior to the underlying stacked balloon section (424) and is shown to be a component that is substantially unattached to that stacked balloon section (except as physically attached due to the pressure of the elastic itself). In any case, the elastic sleeve or layer (442) is not depicted as adherent to or co-extruded with the underlying balloon. Nevertheless, it is within the scope of the description here simply either to co-extrude the elastic sleeve (442) with the underlying balloon (418) if the composition of the balloon allows and the use of the resultant device allows or to make the elastic sleeve (442) adhere to the underlying balloon, e.g., by solvent welding, adhesives, etc.

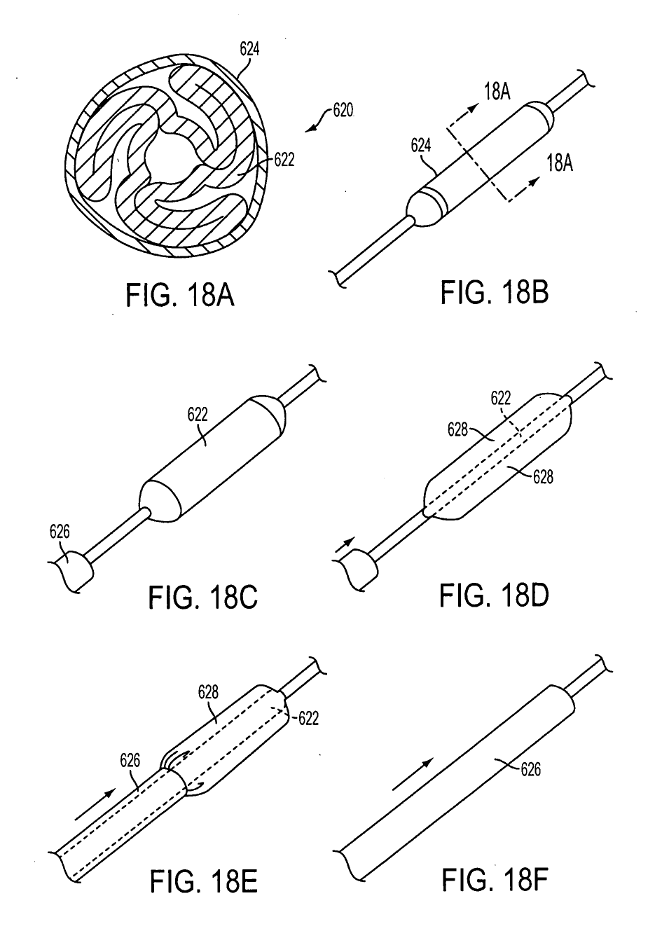

FIGS. 22A-22H show a number of elastic deflation aid variations. These drawings are provided for the purpose of depicting the deflation aid, its positioning and operation, and since those purposes may be explained with drawings that are more schematic in format, these figures may not be to-scale, may exaggerate the size relationships amongst various components, and may omit many obviously necessary components not otherwise critical to understanding of the deflation aid. Furthermore, where the expanding member is not inflated, but is simply expanded in some fashion, the described deflation aids may be considered as a contraction aid and comprise a metallic spring or other similar device for storing tension as described elsewhere herein.

FIG. 22A shows, for purposes of illustration, a variation such as that shown in FIGS. 11A, 11B, and 11C. A controllably expandable assembly (720), in this case a series of inflatable balloons, radially adjacent each other, inner balloon (722), mid-balloon (724), and outer balloon (726). Each of the balloons is fastened, directly or indirectly, to the central catheter shaft (728). Exterior to the outer balloon (726) is elastic deflation aid (730). Elastic deflation aid (730) is shown to be perhaps in slight tension for positional stability, when the underlying balloons are in a deflated condition. As one or more of the underlying balloons are inflated, the elastic deflation aid (730) is stretched and the resultant pressure may be observed, even on gauges exterior to the device.

For instance, during inflation of the underlying balloons, a high pressure balloon may be chosen for outer balloon (726) and be able to operate up to 20 atmospheres (about 300 psig). A balloon pressure of two atmospheres may be sufficient to expand the outer balloon (726) to its operating diameter (in the absence of a lesion to re-form) and to concurrently expand deflation aid (730) to that balloon diameter size. The deflation aid (730) would therefore have a positive pressure of two atmospheres residual pressure available to deflate the balloons situated beneath the deflation aid (730) and squeeze the volume of inflation fluid proximally towards the user for disposal.

Inflation tubing or lumens currently used for balloon catheters are often quite small in diameter and in wall thickness. Vacuum is often used to provide a suction for removal of the inflation fluid through the inflation tubing. Collapse of those thin-wall tubing members during deflation sometimes occurs. In devices without deflation aids, use of such a multi-atmosphere vacuum exterior to the body pulling on a small diameter, polymeric, inflation tubing member might be considered a situational candidate for collapse of the inflation lumen, thus preventing balloon deflation.

However, the presence of a positive pressure created by our deflation aids within the balloon allows use of a vacuum exterior outside the body, and even enhances the flow rate of inflation fluid, but, more importantly, significantly lowers the chances that the inflation tubing will collapse. Our deflation aids should provide a large comparative safety margin.

FIG. 22B shows a variation of the device and the specific physical configuration shown in FIG. 22A discussed just above. The configurations are physically similar; in this way, we discuss by comparison or contrast, placement of our deflation aid components at specific sites in the device system. The variation in FIG. 22B (as well as in FIGS. 22C thru 22H) are each a controllably expandable assembly (732) having, for purposes of this illustration, three radially adjacent balloon members. The depictions in FIGS. 22B thru 22H show the various balloons as being inflated to provide greater clarity in positioning of the deflation aid components.

FIG. 22B shows an elastic deflation aid (734) interior to outer balloon member (306). The deflation aid may be formed in many ways. For instance, deflation aid (734) may be formed along with outer balloon (726) during extrusion step. The adherent elastic deflation aid (734) may be glued to the exterior of a balloon pre-form and the pre-form everted to place the deflation aid on the interior of outer balloon (726). Just as was the case with the variation shown in FIG. 22A, when the various balloons shown in FIG. 22B are to be deflated by releasing the pressure from the fluid interior to these balloons, deflation aid (734) will provide some measure of a positive pressure to squeeze the inflation fluid in each of the three balloons back towards the proximal user.

The variation shown in FIG. 22B is one that may have potential benefit in view of the version discussed just above, in that the elastic deflation aid (734) is in a position in the device that is remote from contact with the body. There may be some regulatory sensitivity towards placement of any latex or latex-like materials adjacent body surface. This variation does not allow such contact.

FIG. 22C shows a controllably expandable assembly (736) having deflation aid (738) mounted exterior to middle balloon (724). In this variation, the balloons are again shown to be completely inflated. If the assembly shown in the Figure is used with both the middle balloon (724) and the outer balloon (726) expanded, upon later deflation of the assembly, deflation aid (738) will act to provide a positive pressure on the fluids found within middle balloon (724) and inner balloon (722). In addition, the collapse of inner balloon (722) and middle balloon (724) under the physical pressure of deflation aid (738), i.e., the resulting disappearance of volume within outer balloon (726), will tend to collapse that outer balloon (726) as well.

FIG. 22D merely shows an alternative positioning of a deflation aid (742) upon an inner surface of middle balloon (724), all making up a part of controllably expandable assembly (740).

FIG. 22E shows a controllably expandable assembly (726) having deflation aid (728) mounted exterior to inner balloon (722).

Common to the operation of each of the deflation aids discussed in this section is the concept that some amount of potential energy is stored in the component, perhaps as tension in the deflation aid component, during the step of inflating the mentioned inflatable members. Although other ways of storing energy distally in the region functionally adjacent the inflatable members are available, elastic tubular members or expandable bands as noted are quite practical. The physical shape of the deflation aid need not be simple tubular cylinder, though. For instance, FIG. 22F shows a deflation aid (730) mounted upon an inflated balloon (732). Deflation aid (730) in this instance, has a cross-section that tapers to one end (734) and is reasonably constant in diameter towards the other end (736). This shape permits the deflation aid to shrink back to its nominally unexpanded size at a differential rate. In particular, such a difference might be valuable in squeezing inflation fluid from a portion of a device that should be cleared from a stent first, e.g., distally in a curving lesion. Similarly, FIG. 22G shows a sequence of deflation aids placed about inflated balloon (742).

FIG. 22H shows a pair of deflation aids (746) mounted upon an inflated balloon (744). The deflation devices shown there have sections that shrink at different rates upon release of interior inflation fluid pressure. Such differential shrinking rates can, for instance, in cooperation with proper placement and selection of specific “winged” balloons, aid in the replacement or refolding of such a non-compliant balloon after use.

Body Treating Devices and System

Included in this description is an overall system for treating a body at a site in the body that needs or requires treating where that treating comprises at least one of: a.) a step of expanding at least one body treating device to a selected diameter and b.) a step of expanding at least one body treating device to a selected length. The system includes at least one controllably expandable assembly, perhaps such as those specifically described here, but in any case configured a.) to be placed in that body at the selected site, b.) to expand at least one of the at least one body treating devices, respectively, to more than one selected diameter or length, and, c.) to be selectable from more than one diameter or length without having to be removed from the human body. The system is especially configured for expanding at least one body treating device and optionally includes the device.

The body treating device includes devices that, upon expansion, at least begins to perform a treating function. It may be, the treating devices specifically described herein, e.g., stent, stent-grafts, grafts, various sleeves, movable sleeves, stents mounted upon sleeves, cutting balloons, stents mounted upon movable sleeves, constraining sleeves, and others not specifically shown herein but would be operable with the controllably expandable assembly.

The controllably expandable assembly itself may be any of the configurations and variations described here and specifically includes variations such as those having: a plurality of expandable members, a plurality of inflatable members, a plurality of inflatable balloons (including those where at least two of the balloons are radially adjacent each other, where at least two of the balloons are axially displaced from each other, and combinations including those variations).

Balloons and Shape Control Elements

The balloons and expandable members described herein may be made of the usual materials otherwise found in medical balloon devices currently used in medical treatments. Such balloons are often divided into three groups: compliant balloons, semi-compliant balloons, and non compliant balloons. The definitions of these balloons and materials are not rigid nor drawn with a bright line. That is to say that “non compliant” balloons indeed have some measure of compliance to the anatomical lumen, once expanded. Balloons comprising certain types of elastic material may reach a point upon extensive expansion where they are no longer capable of compliance with an exterior force or surface. Indeed, compliant balloons may not shrink to their previous shape after such a hyper-inflationary exercise. Nevertheless, there are approximate understandings in the medical arts relating to such terminology and despite the vagaries of use in such technology, we are using those words in the same approximate ways that the current users in this field use those terms. Additionally, the materials used in forming the various balloons suitable for the described device may be characterized as elastic, elastomeric, non-elastic, and the like. Since these terminologies themselves are often considered to be regions of a continuum, we will use those words in a sense as they would be currently used in the field of polymer engineering. The materials making up the balloon will also be mentioned in a generally descriptive fashion in the way those words would be used in colloquial, technical discussions.

That having been said, examples of materials useful in making “elastic” balloons include various polymeric materials used currently in compliant medical balloons, e.g., elastomeric membranes having a high degree of linearity (non-plasticity) for a wide range of stress and strain values. Such materials include various Silicones, latex, Kraton, various thermoplastic elastomers (TPE's) particularly styrene-ethylene/butylene-styrene block copolymers (SEBS)-based TPE's (such as C-FLEX), polysiloxane modified SEBS and their associated families, polyvinylchloride (PVC), cross-linked polyolefins such as polyethylene, and various polyurethanes. Examples of materials used in making “inelastic” or noncompliant balloons include many of the polyamides (e.g., the Nylons), thermoplastic polyamides, polyesters, polyphenylene sulfides, and polyethyleneterephthalate (PET). PET is especially interesting due to its capacity for easy production of very thin wall balloons.

The balloon material may be selected or treated to allow the chosen inflation fluid to permeate through the balloon wall. The treatment may be chemical or physical. This ability may be useful when, for instance, the fluid is used to treat a medical problem on the bodily structure to which the balloon is applied.

The polymeric material making up the balloons (and other components and sub-assemblies of the system) may further comprise one or more solid radio-opaque materials such as particles of tantalum, gold, tungsten, platinum, tantalum oxide, barium sulfate, and their mixtures when the designer sees the need for an amount of radio-opacity.

Although the scope of balloons used in the described device include variously balloons that expand when a fluid in imposed on the interior of that balloon, this variation device is especially useful when employing balloons comprising elastic materials. One benefit of using these type of materials is the functional ability of such a balloon to return its original profile after the inflating material or fluid has exited the balloon and to do so with great speed. This allows the core guide member to proceed distally down, e.g., a vascular pathway, with greater ease than were one to employ a noncompliant balloon that would simply fold after deflation. Such folded balloons are simply less suitable in certain circumstances in medical procedures, for additional or subsequent treatment, especially where the treatment is more distally located in the particular anatomical system. That is to say, for instance, should a physician desire to place a stent more proximally in the neural vasculature and thereafter to place additional stents more distally in that same blood system, a lower profile is highly desirable for the steps of implanting those additional distal stents. Because of the narrowness of the neurovascular pathways, any advantage in lower profile is a significant advantage. In such medical procedures, an elastic balloon is highly desirable as a matter of achieving such a lower profile.

Benefit may also accrue in this variation when an outer elastic balloon is situated exterior to an inner non-compliant balloon or balloons, as explained elsewhere herein. In particular, the outer balloon causes the two balloons to deflate together with positive pressure on the inflation fluid.

On the other hand, there are instances in which the desirability of having a non compliant balloon produced from a material having the capability of accepting very high inflation pressures is a better answer, e.g., when one has calcified plaque on the arteries and one wants to exert very high pressures without increasing the size of the balloon beyond a pre-specified diameter. Similarly, for placement of stenting devices distally in an anatomical system where the stenting device is very sturdy and hard to implant, the better answer is to use a non-compliant balloon to effectively place the stent. However, once that inelastic balloon is inflated and then deflated, it unfortunately presents the specter of a residual, larger profile than would the same balloon made from an elastic material. This presents an opportunity to place an outer elastic polymer balloon exterior to an inner non-compliant balloon and use the outer balloon as a folding aid, much in the same way as the elastic covering (442 in FIG. 11A) is used to help compress the balloons, as described there.

The multi-layer balloons used in the described systems allow tighter optimization of the physical properties of the balloons. For instance, only the outer balloon need have a balloon material that is scratch resistant; the remainder of the balloons, protected by the outer balloons may be thinner than the norm and need not be as “tough” as the outer polymer covering.

Further, inflating the inner-most balloon at a high pressure in one of the variations above (even if the balloon is compliant) is safe and effective because the outer uninflated balloons serve both as diameter limiting bands and as safety coverings.

When inflating balloons, we have sometimes observed that it would be desirable to “focus” the radial expansion of the balloon via the use of expansion control members situated at one or the other ends of the balloon or both. The expansion control members are useful in conjunction with balloons made from other materials, variously compliant, semi-compliant, and non-compliant, although their use is typically more advantageous with the elastic balloons.

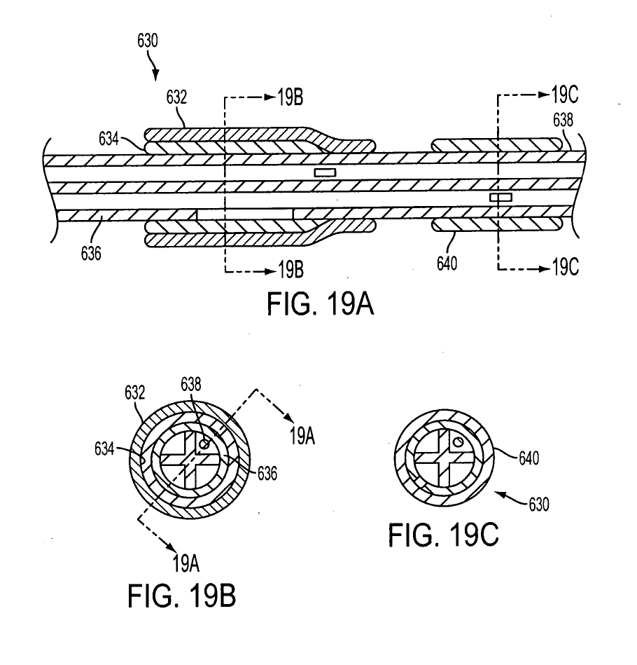

FIGS. 23A through 23D show, albeit in a somewhat exaggerated fashion, the process of expanding an elastic balloon using the described system, the steps are instructive in explaining the use and desirability of such expansion control members.

FIG. 23A shows a simple variation of the described device (750). Shown in FIG. 23A is an expandable, elastic balloon (752) and the constraining member (754). A guide tip (756) is also shown. In FIG. 23A, the constraining member (754) has been slid onto balloon (752) until approximately one-third of its longitudinal length has been situated beneath constraining member (754). In FIG. 23B, balloon (752) has been partially expanded and forms a reasonably spherical shape.

In FIG. 23C, balloon (752) has been expanded and the proximal end of the balloon is beginning to splay the distal end of constraining member (754) and indeed is beginning to roll proximally (758) over the end of the constraining member (754).

In FIG. 23D, balloon (752) has rolled proximally over the distal end of constraining member (754). In the situation shown in FIG. 23D the surface of the balloon exterior-most to the device (750) is beginning to axially expand in such a way that it may not be especially suitable for performing those types of tasks where the overall length of the balloon is to be narrow and controlled. That is to say, in some procedures for which the described device may be used, the user might wish to have a balloon length of 0.200 inches because that's the length of a particular implant to be deployed. Additional extension longitudinally of the balloon might interfere with placement of other, adjacent stents on a delivery device, or injure healthy vascular tissue. Consequently, in the chosen procedure, the axial length of the expanded elastic balloon should be controlled.

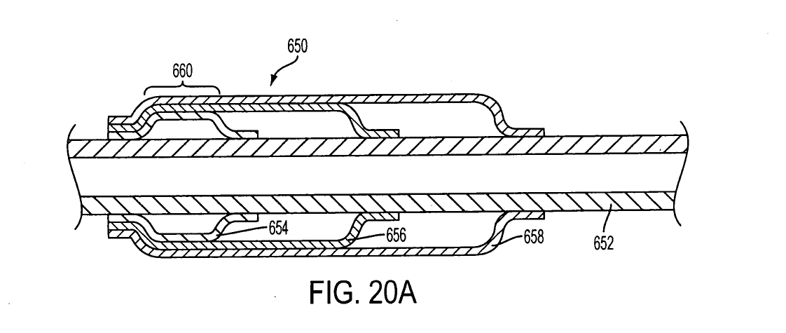

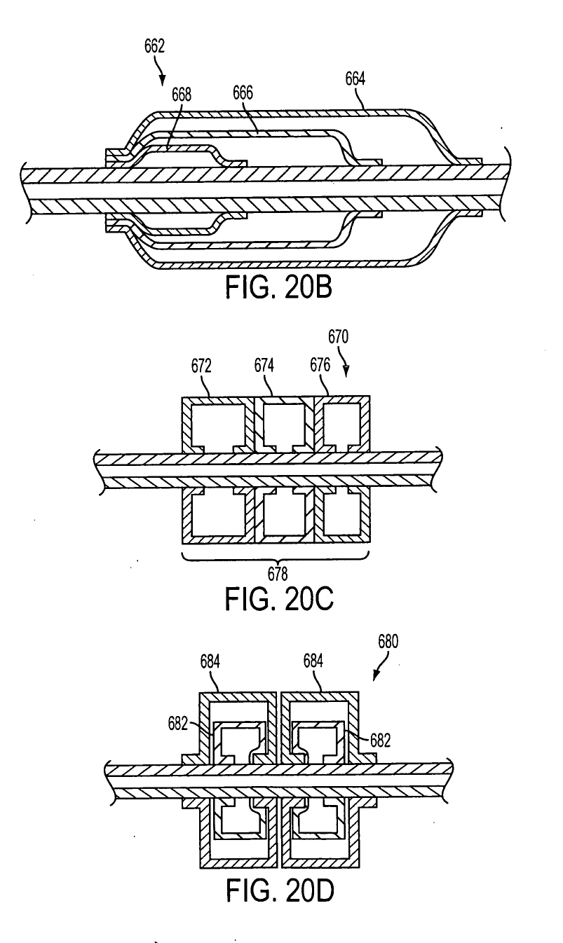

FIG. 24A-24C show, in concept, the presence of at least one expansion control member (760) situated distally upon slidable constraining member (762). As balloon (752) is expanded in FIG. 24B, the expansion control member (760) expands at its expansion end (764) along with balloon (752) and controllably presses upon balloon (752) to maintain the outer profile of balloon (752) in a position focused beyond the end of expansion control member (760). The step shown in FIG. 24C depicts balloon (752) at its maximum practical expansion and with the expansion end (764) of expansion control member (762) directing the balloon away from the end of the expansion control member (760) located remotely from balloon (752). Additional details of a number of examples of expansion control members may be found just below. FIGS. 25A and 25B show the use of distal expansion control members (768) located distally with respect to balloon (752) as well as proximally (760) as discussed just above.

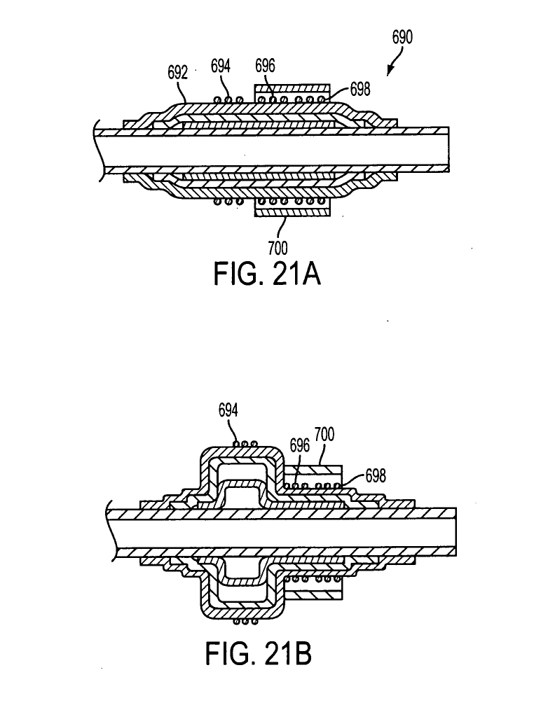

FIGS. 26A, 26B, 27A, 27B, 28A, 28B, 29A, and 29B show various examples of the expansion control member and its operation.

FIG. 26A shows a side, cross section of an expansion control member (770) having a plurality of slots (772) situated in the outside surface and extending axially down the member (770). As the balloon (not shown) expands, the end of member (770) expands as well, generally about hinge point (774). As is shown in FIG. 26B, the expansion widens the portion of the slots adjacent the balloon. The expansion of the wall slows and stops as the slots widen to their maximum spread when the balloon ceases to expand. This conformation allows control of the shape of the balloon as it expands and focuses its expansion distal to member (770).

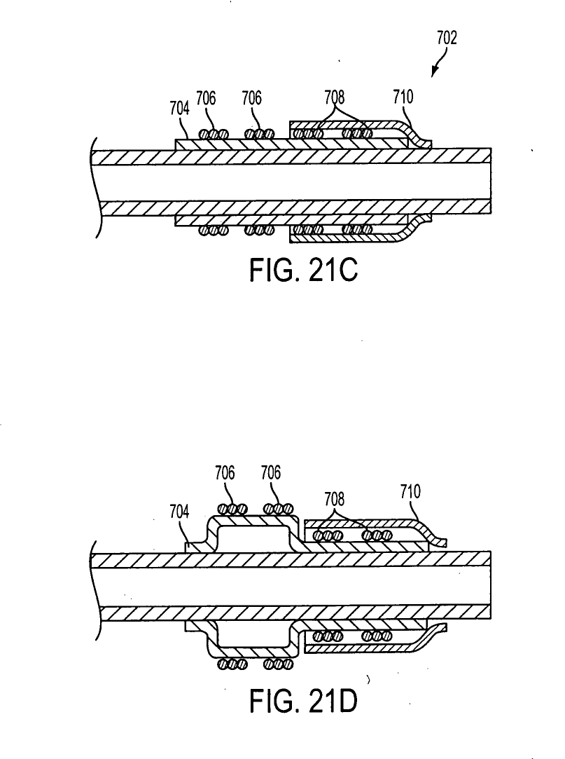

FIGS. 27A and 27B show a similar arrangement, sans slots, in which, e.g., a material that is more compliant than the shaft but less compliant than the balloon material used in the production of member (776) or at least its distal portion. A cinch (776) is present at the proximal end of the zone forming the expansion control member. Expansion of the expansion control member (776) about cinch (778) is shown in FIG. 27B. Usually, the material (776) is less compliant than the balloon.

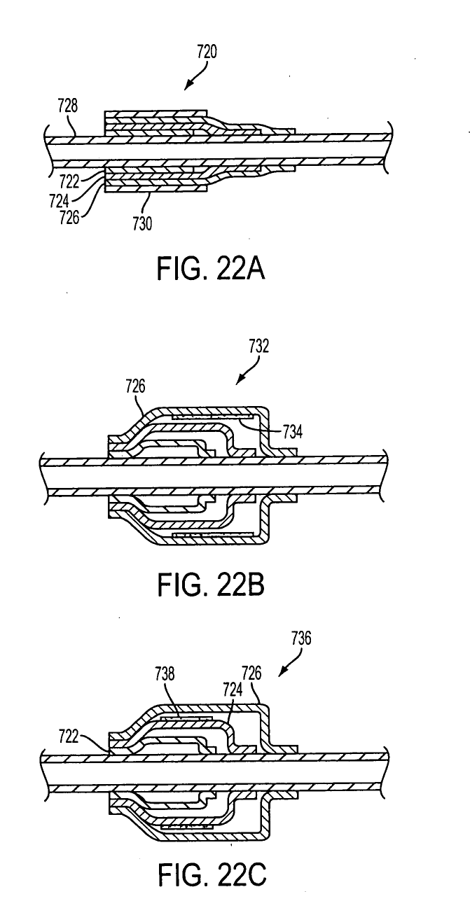

FIG. 28A shows a member (780) having an expansion limiter (782) present in the expansion control member zone. The expansion control limiter (782) is configured with a convoluted form such that when the balloon expands and the limiter is re-formed as is shown in FIG. 28B, that limiter (782) has been stretched to remove its convolutions. The expansion limit of the expansion control member (780) is controlled by the length of the wire forming expansion limiter (782). The limiter (782) itself, may be placed at any point along the axis of the expansion control member (780), but is often more effective as a specific size stop when placed near the end of the limiter adjacent the balloon.

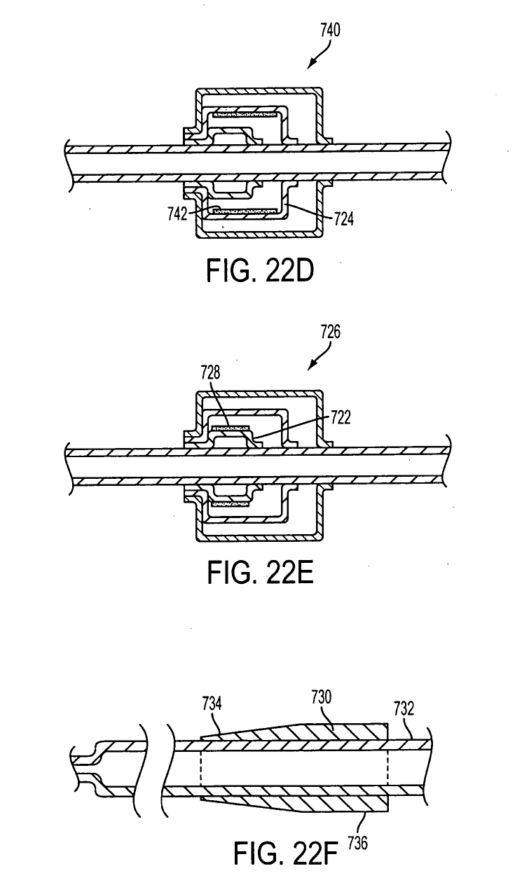

FIGS. 29A and 29B show another variation of the expansion control member (784) in which functionally longitudinal stiffeners (786) are situated in a generally longitudinal fashion distally upon member (784) in such a way that the expansion control member is conically shaped upon expansion as shown in FIG. 29B.

As noted above, these described expansion control members may be formed in such a way that they: a.) comprise the distal portion of the constraining members (described elsewhere), b.) are an integral portion one end or the other of a balloon member as described herein or c.) may be independently, non-integrally, placed at the proximal or distal ends of a balloon or balloons.

Implant Delivery Components

One of the substantial medical procedures that may be carried out using the described system is the intricate placement of implants, such as stenting devices, using the variable length of balloon described herein. In part because of the size of certain variations of the described system, the system is amenable to the implantation of multiple stents without withdrawal of a component of the system from the human body. It is often the case in procedures used today that after but a single stent is introduced to a treated site in the body, introduction of another stent is accomplished only after withdrawal of the first placement component that included a stent and reintroduction of another stent-containing component, i.e., a balloon with stent on it. As will be discussed below, our system is suitable for placement of a number of stents without withdrawal of the stent carrying cartridge.

The form of the implants that may be delivered by use of the described system is quite varied. The implants may be stents or other devices having stent-like structures or functions (e.g., closures for aneurysm mouths). The form is not particularly important and may be of any desired shape or configuration. The implant, e.g., stent, may be expandable, balloon-expandable, or self-expanding. The system may be assembled in the field, e.g., in an operating room, or may be pre-assembled.

In addition, the implant may be radio-active or drug-eluting, e.g., contain a biologically active material. Many of the biologically active materials discussed herein are found in so-called “drug-eluting stents,” stenting devices that may be implanted using the system described here. The implant or stent may comprise at least one biologically active agent, such as a releasable biologically active agent selected from the group consisting of anti-proliferation agents, anti-inflammatory agents, antibiotics, and immunosuppressants. Immunosuppressants include Sirolimus (Rapamune®) previously known as rapamycin, Everolimus formerly known as mycophenolic acid, and tacrolimus (Prograf). Other immunosuppressants include cyclosporins (e.g., Neoral, Sandimmune, SangCya), azathioprines (e.g., Imuran), and corticosteroids such as prednisolone (Deltasone, Orasone).

Particularly useful biologically active agents are those selected from the group consisting of paclitaxel, methotrexate, batimastal, doxycycline, tetracycline, rapamycin, actinomycin, dexamethosone, methyl prednisolone, nitroprussides, prednisolone, estrogen, estradiols, and their mixtures.

The deployed implant may be of a design that is smaller prior to and during delivery and then larger after implantation. The implant design may be used to provide or to maintain patency in an open region of an anatomical structure, or to occlude a site, or to isolate a region (e.g., to close an aneurysm by blocking the aneurysm opening or neck by placement of an implant in an adjacent anatomical structure such as an artery or gastrointestinal tubular member), or to hold a number of occlusive devices (e.g., coils or hydratable polymeric noodles) or compositions at a site to be occluded or supported. The implant design may be one that collects embolic material in a blood stream. The system may also be employed for local medication or drug delivery via implant delivery into solid organs or tissues including skin, muscle, fat, brain, liver, kidneys, spleen, and benign and malignant tumors.

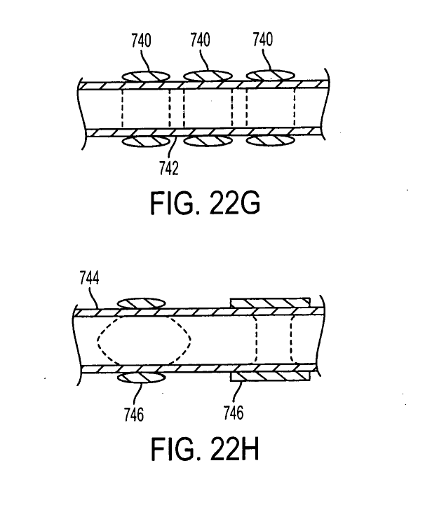

FIG. 30 shows in schematic form, a stent delivery and deployment tool member (800) similar in design to the implant or stent delivery component (112) shown in FIG. 1A. It includes an expandable delivery sleeve (802) and, in this variation, a sequence of stenting devices mounted on and upon expandable sleeve (802), each independently deployable using an expandable balloon situated in a passageway interior to that expandable sleeve (802).

FIG. 30 further shows a position control member (806) attached in some fashion to expandable sleeve (802). The position control member is a component used primarily to properly situate the sleeve by control from outside the body, so that the selected stent is deployed in the treatment site desired by the physician. Position control member (806) typically is a tubing member having an interior passageway allowing passage of the core guide member or balloon catheter with its included integral balloons or balloons and the complementary constraining member.

Again, the implant delivery device (800) and other variations shown here typically are of a type that are able to deploy one or more of the stenting devices individually or in tandem. The stenting devices may have different diameters (as found before delivery and after implantation) as well as different lengths. They may be of differing (or the same) stiffness. On the stenting or implant delivery device or sleeve, they may be variously balloon-deployable or self-deploying, even on the same sleeve.

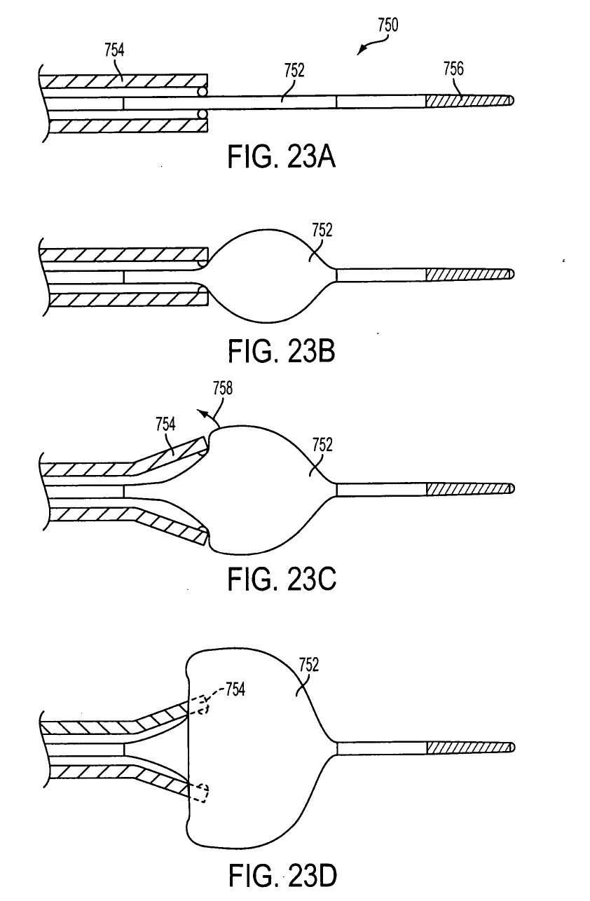

FIG. 31 shows another variation of the implant delivery device, again having an elongate position control member (806) with passageway within and a stent delivery sleeve (802). In this variation, the implants or stenting devices or stents are longitudinally of differing sizes. In the variation shown in FIG. 31, the most proximal stent (812) is short and the next more distal stent (814) is longer. The next more distal stent (816) is longer still and the longest stent (818) is then just distal. Another smallish stent (820) is the most distal of the group mounted on delivery sleeve (802). Stents of differing or similar or the same size (length or diameter) may be placed on the stent delivery sleeve during manufacture or during the conduct of the procedure for deploying the stents or at any appropriate time therebetween, e.g., just after dye injection and pre-percutaneous intervention.

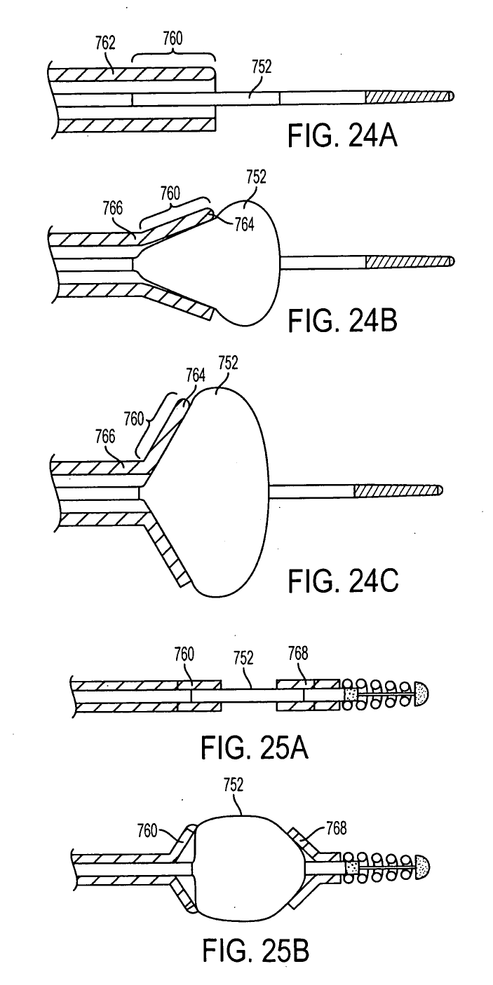

FIG. 32 shows the variation of the implant delivery component (822) and a number of sleeve subcomponents (824, 826, 828). The expandable sleeve subcomponents of (824, 826, 828) are independent, joinable to other components in these tool sets, and may be joined end-to-end in a configuration suitable for deployment of the cooperative stents (830, 832, 834). They may be joinable to form an implant delivery member with inclusion of the position control member (836). After fixably assembling the various components of implant delivery (800), the component is slid into the body in the same way that those described just above are introduced.

The delivery sleeve comprises any expandable braided, woven, or co-wound structure, commonly columnar or tubular in general form, and typically will be made up of filamentary or ribbon-like materials. Often, the materials will be metallic or polymeric in composition and will be of a size and flexibility such that the sleeve is expandable on upon imposition of balloon pressure on the sleeve's interior and of a material that will return to its original shape upon relaxation of that balloon by deflation. The delivery sleeve, may, if so desired, be of one or more diameters.

The delivery sleeve may also comprise an expandable elastomeric sleeve, commonly columnar or tubular in general form, and suitably formed in such a way that it will support and deliver stents in the noted fashion.

FIG. 32A shows a stent delivery sleeve (840) having a generic single stent (842) placed thereon and a position control component (806) extending proximally from sleeve (840). In this variation, the sleeve (840) is braided from a plurality of wires (844). A cross section of wire (844) is shown in FIG. 32B. Shown interior to sleeve (840) is balloon (848) mounted on a core guide member. Although any reasonably strong, springy material may be used in this sleeve, excellent results are achieved when the wire (or ribbon discussed below) is a substantially non-plastic metallic alloy such as a superelastic alloy. Again, nitinol is a fine choice for the filaments making up the sleeve.

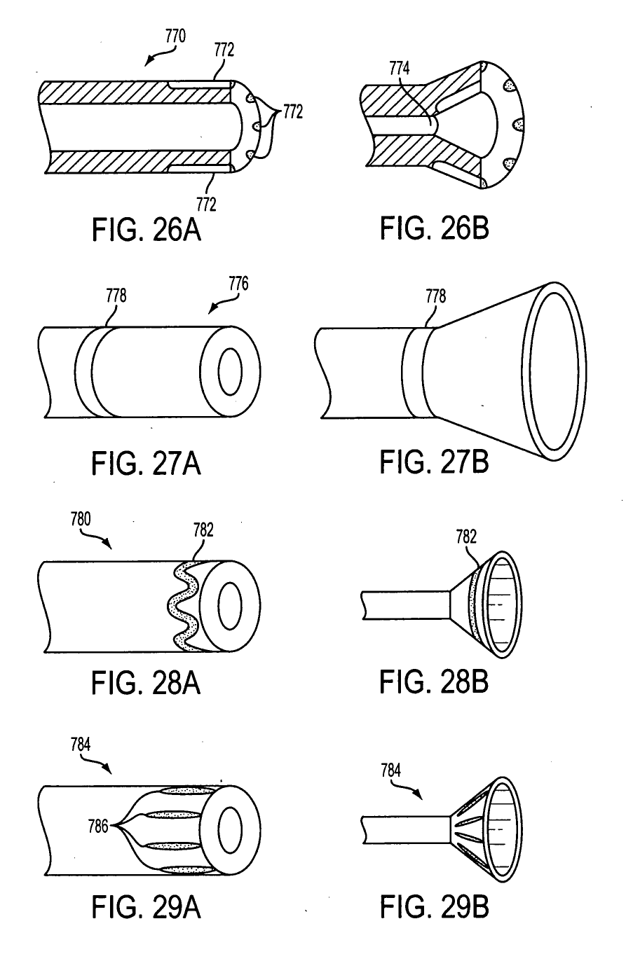

FIG. 33A shows a similar configuration with mounted stent (842). The sleeve variation (850) found here is braided from ribbon-like filaments. FIGS. 33B and 33C show examples of such a ribbon cross section. FIG. 33B shows an oval ribbon cross section (852) and FIG. 33C shows generally rectangular ribbon cross section (854). Woven or braided sleeves such as shown here are particularly resistant to kinking during the steps of placement and deployment in a turn in an anatomical lumen. Also found in FIGS. 32A and 32B is an optional covering layer (846). The covering layer (846) is optional and may be present for a variety of reasons depending upon the service into which the described system is placed and its accompanying needs and requirements. For instance, in some procedures, it may be desirable to have the stent somewhat adherent to the delivery sleeve. Inclusion of a tackifying layer including, perhaps, a fibrin-based glue might therefore be desirable. Another example is providing an elastic material layer in such a position to provide a barrier between the sleeve and the stent and to allow for smooth separation of the stent once deployed. Such a barrier will provide a prophylaxis against potential physical damage caused by mechanical movement of the sleeve against the deployed stent.

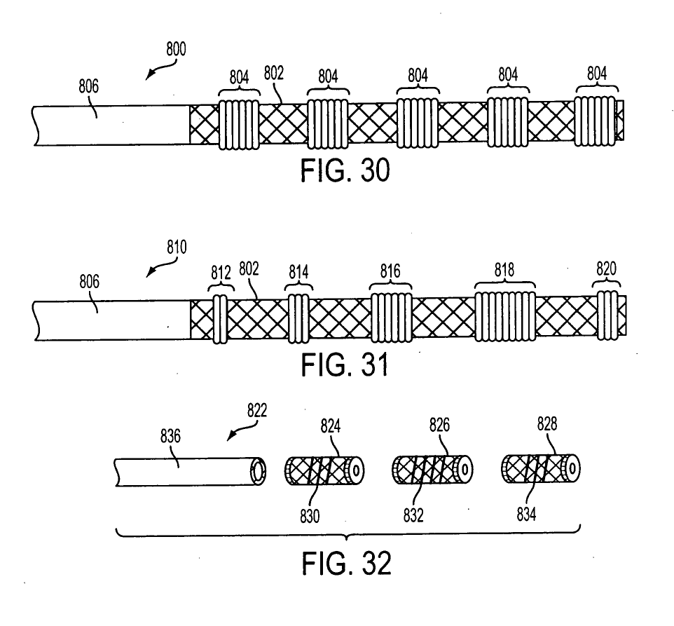

FIG. 34A shows a further variation of the implant sleeve assembly (856). This variation of the stent sleeve assembly (856) comprises an elastic sleeve (858) having more than one stenting device (860, 862, 864) mounted thereon in such a way that an inflatable balloon, described above may fit into the interior passageway (866), the balloon be adjusted to an appropriate axial length, and expand to deploy a single stent without substantially affecting the positioning of one or more adjacent stents. Of course, it is within the scope of this device, that the balloon be expanded axially in such a fashion that more than one stenting device be deployed.

FIG. 34B shows the elastic sleeve (858) and stent (862) deployed. A balloon is present within the bulge (868) shown beneath stent (862). The guide member (870) is shown extending from the sleeve (858). Note that stent (860) and stent (872) have not been affected by this implantation step.

Although elastic sleeve (858) may comprise simply a polymeric tubing without additional reinforcement, in many instances, it is likely that additional features would be appropriate for easy operation. For instance, a tackifying composition may be desirable to maintain the stents in their position during placement of the sleeve (856). Longitudinal reinforcement to potentially prevent axial expansion or contraction during placement of sleeve (856) and ease of expansion without affecting neighboring stents may be desirable.

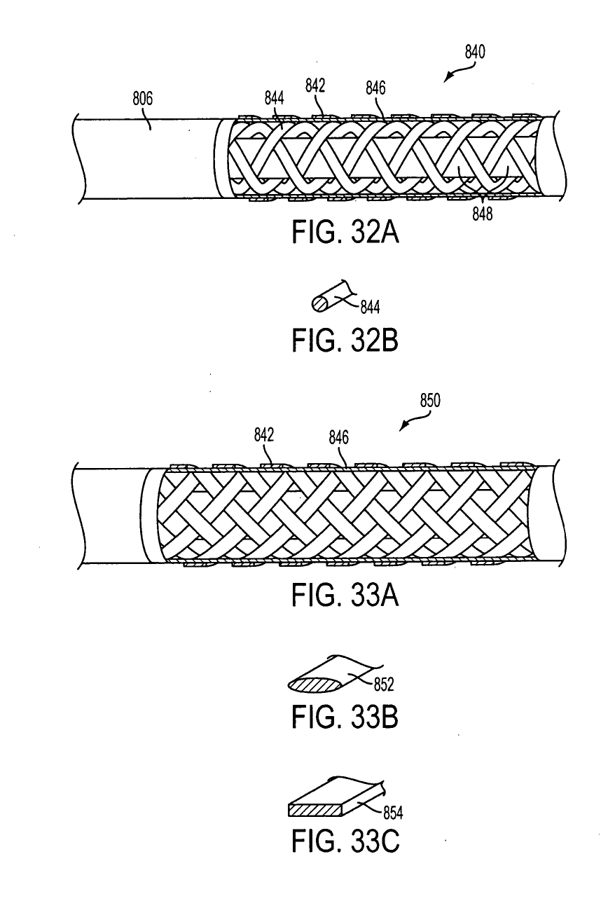

The usefulness of such a deployment sleeve is not limited to the deployment of implants that are balloon-expandable. FIG. 35A shows another stent deployment sleeve (858) again attached to a position control component (880) and, in this variation, a self expanding stent (882). Over all of this is a retractable sleeve (884) holding the self expanding stent (882) in place during the steps of delivering the stent (882) to the region where needed. FIG. 35B shows withdrawal of sleeve (884) and self expansion of stent (882) into position while leaving the expandable sleeve (878) in place.

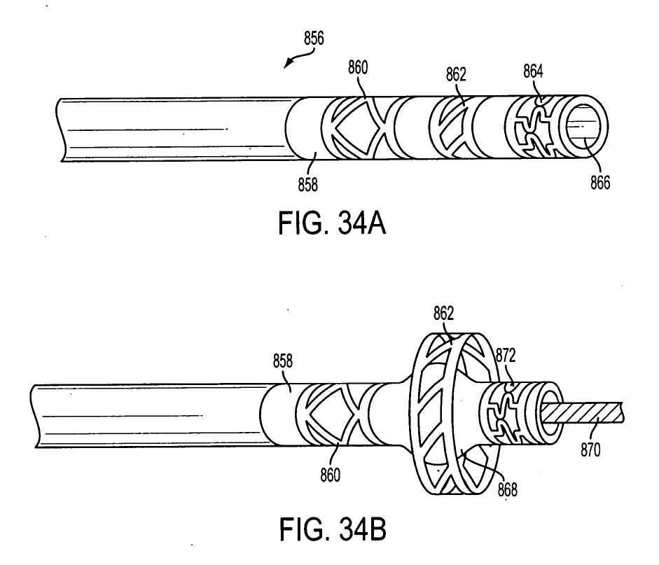

FIGS. 36A-36C show simple deployment of a single stent, a stent that is most proximal on the delivery sleeve and is balloon expandable. This example shows initial placement of a more proximal stent from the delivery sleeve. This instance merely illustrates the flexibility of the device in allowing choice of a stent from the inventory on the sleeve, that matches the size, the length and diameter, of the treated lesion. Other stent placement choices may include to first utilize a more proximally placed stent to allow subsequent placement of stents situated distally upon the sleeve upon lesions more distal in the vasculature where extra distal space in the more distal vasculature is not available. Or, another choice would be to choose first to deploy the more distal stent if required. The takeaway points are that the choice remains in the hands of the user and that the device permits placement of multiple stents of varying length and diameter without removal of the balloon catheter or core guide member from the body.

FIG. 36A shows an implant delivery component (890) having expandable sleeve (892), a position control member (894), and several stents (896) mounted on sleeve (892). As noted above, both sleeve (892) and position control member (894) have passageways that are generally aligned in such a way that a balloon of the type described here may be passed through the passageway and inflated in the region beneath the desired stent. FIG. 36B shows expansion of the balloon in the most proximal region of the expandable sleeve (892) thereby expanding the diameter of the proximal stent (896). FIG. 36C shows the return of the stent delivery sleeve (892) to its original shape leaving the then-expanded stent (896) at the larger diameter. Note that the stent adjacent the deployed stent was not affected by the deployment of the proximal stent.

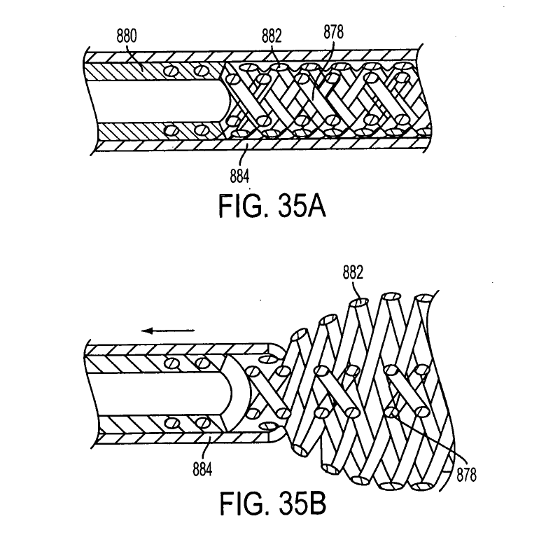

FIG. 37 shows the use of the expanding sleeve (900) with a stent graft (902). As is the case with stent-grafts, the stent-graft (902) depicted includes an interior stent (904) and an exterior graft (906). Delivery of stent graft (902) with the expandable implant sleeve (902) is a bit more problematic, in general, than implanting balloon-expandable grafts since most fabric grafts have but a single predetermined diameter and are designed to fit in a specific anatomical site of a specific diameter. Further, some planning is needed to prefold the graft (906) in a way that will allow passage of the stent graft (902) to the needed site. Our delivery system works quite well in delivering such grafts. In any case, common graft materials are also suitable for use in this device. Materials for such a graft include, in particular, expanded polytetrafluoroethylene (ePTFE) such as GoreTex, and Dacron, etc.

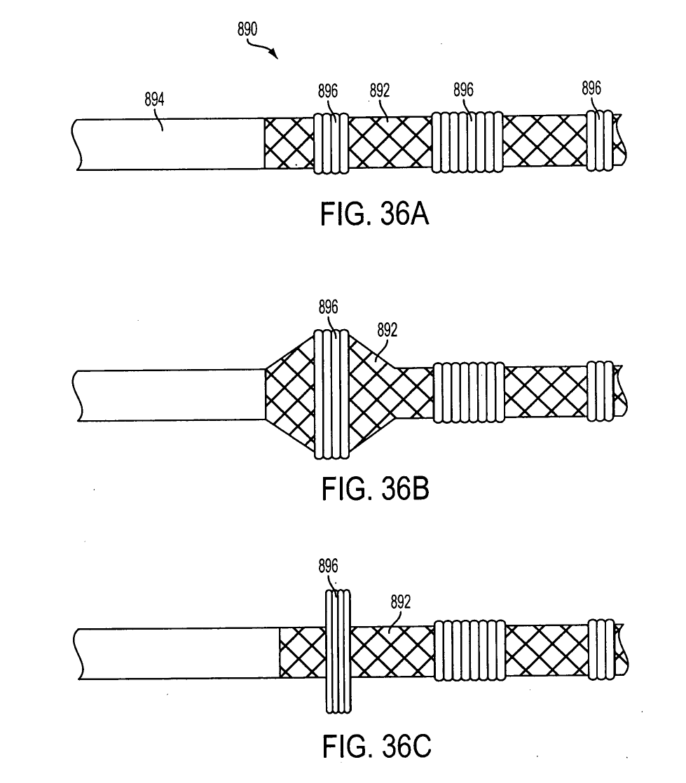

It should be apparent that each of the stent sleeve designs discussed herein is useful either in conjunction with the core guide member-based variations of our described system or in conjunction with our balloon catheter-based variations. FIG. 38 shows portions of the system, specifically having a balloon catheter (908) with balloon (910), and constraint member (912). Overlying both the catheter (908) and the depicted constraint member (912) is stent sleeve (914). Stent sleeve (914) carries on its outer surface, a number of stents (916, 918, 920, and 922) of various configurations and sizes. Balloon (910) is in position and is partially constrained by constraint member (912) in such a way that a controlled portion of stent sleeve (914) is expanded so to deploy stent (922). Operation of the stent sleeve (914) in this variation is quite similar to its operation with regard to the core guide member discussed above.

Although delivery of stents using a stent delivery sleeve as discussed above is one of the more facile ways of providing implants, the described system is not limited to the use of the stent delivery sleeve. In particular, stents may be placed directly upon the various balloons and the system used for direct stenting. This system may be used for such direct stenting either alone or in combination with other stenting perhaps using the stent delivery sleeve described here. By “direct stenting” is meant the implantation of a stent upon a treatment site, e.g., a lesion, without first dilating the site.

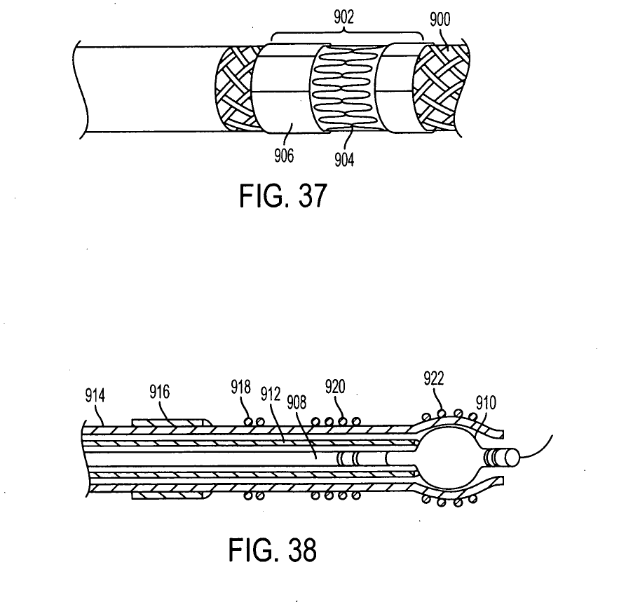

FIG. 39 shows a cross-sectional, side-view of a direct stenting device based upon the core guide member variation of the described system. FIG. 39 shows but a single stent (930). Other methods both for direct stenting, e.g., using the stent delivery sleeve without first dilating, and for stenting after dilation are discussed elsewhere in this disclosure. This variation of the system employs a stenting device (930) mounted over the balloon (932). Also present is a constraining member (934) but, because the stenting device is often initially deployed, it typically would be used only after the delivery of this initial stent (930). In this variation, the balloon and the overlying stent would be placed in the vicinity of the malady to be treated, e.g., a stenosis in an artery, and once there, the balloon expanded and the stent deployed.

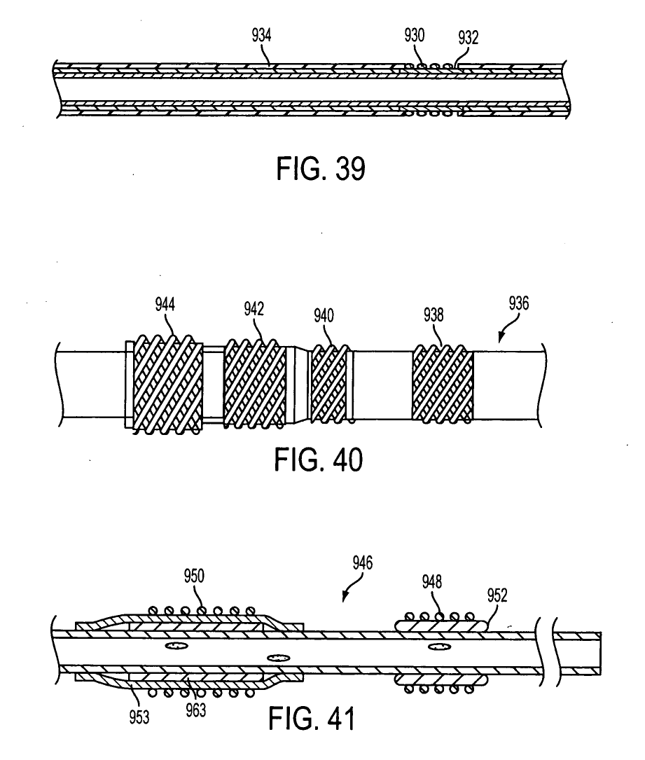

FIG. 40 shows a side view of a core guide member variation of the system (936) having a number of independently deployable stents (938, 940, 942, and 944). In this variation, each of the stents is placed over an individually activatable balloon operating in the fashion shown in the discussion relating to FIGS. 11A and 11B. However, the various balloons are displaced longitudinally along the core guide member in this example.

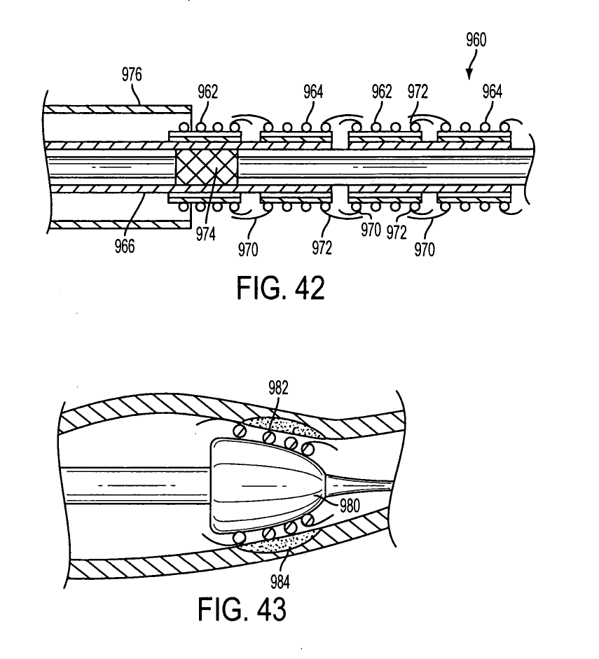

FIG. 41 shows a cross-sectional view of a balloon catheter (946) also having multiple stents (948, 950) mounted respectively over a distal single balloon (952) and a more-proximal set of balloons (953). The two stacked balloons (953) are independently inflatable to allow choice of the final diameter of the outside stent (950) shown there. The details of the lumens have been omitted from this drawing, but the two stents (948, 950) are independently deployable from this configuration.

The variations shown in FIGS. 40 and 41 allow a physician to choose the length, number, and diameter of the stent or stents to be delivered without removing the device from the patient's body using “direct-stenting” procedures. The variations shown in FIGS. 11A, 11B 11C, 30, 31, 32, 32A, 32B, 34A, 34B, and 38 and others as desired permit such flexible stenting using stent delivery sleeves for both direct stenting as well as stenting after pre-dilation.

Many of the commercially available stent configurations, as might be used in the system variations mentioned just above incorporating isolated stents, as a practical matter are of but a single pre-expanded size. That is to say that stents of certain nominal sizes, e.g., 2.5, 3.0, and 3.5 mm, are actually each the same pre-expanded stent. The fact that there is a variation in the finally deployed stent diameter is the result only of the difference in deployment balloon diameter. However, when such stents are used with one of the systems we have described here using stacked balloons, since we can employ stacked balloons, perhaps non-compliant in nature, having the specific nominal sizes of 2.5, 3.0, and 3.5 mm, use of our system by inflating the inner balloon would result in a 2.5 mm diameter stent. Inflation of the middle balloon would provide a stent of 3.0 mm diameter. Inflation of the outer balloon would expand the stent to 3.5 mm. Indeed, if the choice of a 2.5 mm final diameter proved to be too conservative a choice, the physician would be able to revise the diameter immediately, without changing the catheter, ideally without even moving the catheter, simply by changing the choice of balloon to inflate. Consequently, in the example above, a set of three independent, effective catheter sizes is available using our catheter and the choice may be made on the fly. This is facilitated by the use of specific valve configurations that allow adaptation and selection of balloon size. Examples of these valve configurations are shown elsewhere herein.

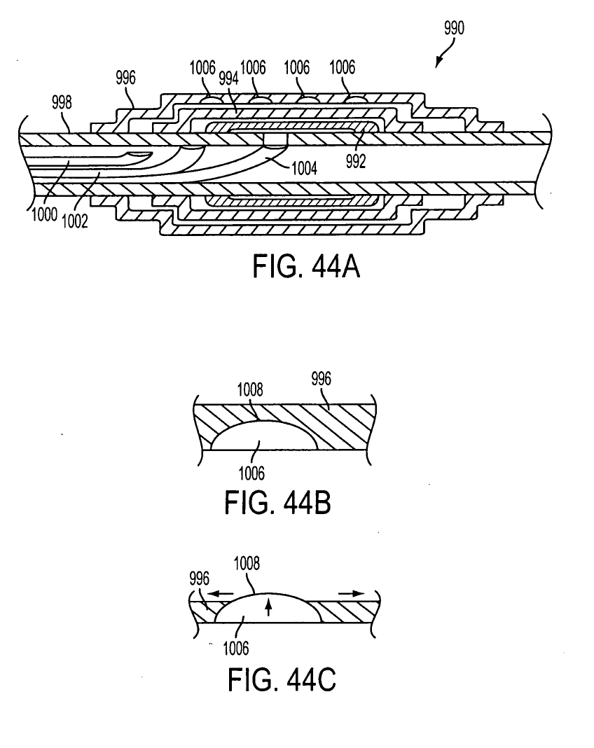

Long stents are desirable in that they may be made to extend over the ends of a stenosis and prevent restenosis with but a single placement step. The system variation shown in FIG. 42 permits a physician to deploy a series of linked stents that, when deployed, act as a single long stent. Using our systems and the component shown in FIG. 42, the physician may select the effective length of the deployed stent assembly either a.) by selecting a long balloon and using such a long balloon to expand all of (or many of) the linked stent sections to produce a linked, deployed stent linked, or b.) by selecting a short balloon and using the balloon to expand (perhaps serially) sections of the stent to produce a series of expanded stent sections. Similarly, one may achieve different stent lengths by using the sliding constraining member, over and under the stent delivery sleeve as shown in the Figures. In such a variation, the variably-sized stent is mounted upon the catheter itself. The stent's size, i.e., the deployed length, is then dictated by the position of the constraining member over the radially stacked balloons.

Specifically, FIG. 42 shows a stent sleeve assembly (960) discussed in more detail below, having a series of interlocking stents (962, 964) mounted on the sleeve (966). The stents are configured to release either singly or as a set. The stents (964) having the outer interlocking members (970) may be released one-by-one since when the balloon expands beneath the stent (964), it is free to expand without interference from the adjoining stents. Conversely, the stents (962) having the inner interlocking members (972) cannot be released one-by-one (unless the adjacent members are gone) since when a balloon expands beneath them, those inner interlocking members move radially (or outwardly) and catch whatever is in the way. If there are adjacent stents having the outer interlocking members, “whatever is in the way” includes the adjacent stent and a group of stents is moved together. If there are no adjacent stents (974) having outer interlocking members, of course, the stents (962) having an inner interlocking members (972) may be then implanted one-by one. A balloon (974) is available on a core guide member to expand the stents as needed. Also shown is a constraining sleeve (976) that may be used to prevent or to limit the expansion of a stent, if so desired during a procedure.

One benefit of length adjustable devices is that by independently inflating the underlying stacked balloons, choosing an inflated diameter, and moving the constraining sleeve (where needed), or advancing the catheter under the sleeve, the user has the ability to place stents of diameters and length chosen in “real-time.” As may be apparent from other areas in this disclosure, the constraining sleeve may be placed beneath (inside of) or above (exterior to) the stent sleeve.

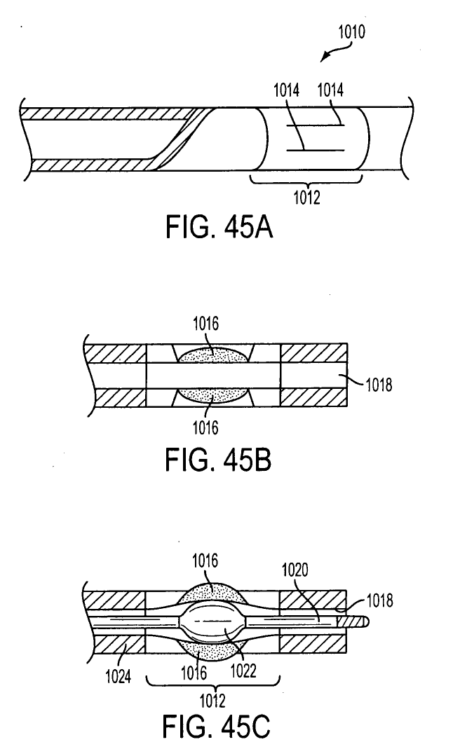

FIG. 43 shows a balloon (980) that is tapered to help assure that the deployed stent (982) is well-placed against the vascular lesion (984). This tapering may be the result of use of a non-compliant balloon having that specific shape, or by use of a caul (discussed below) or by tailoring a multi-balloon catheter (as discussed earlier) to achieve a conical or tapered shape. It should be noted that each balloon shown in this document need not have a constant diameter. The diameter and profile may be chosen as needed to achieve a specific purpose. The taper shown in this FIG. 43 is especially useful in expanding stents in the coronary arteries because those arteries taper at a high rate along their length, often as much as one/tenth (0.1) millimeter per centimeter of length.

Many of the devices and components described herein may be used to achieve a stent having such a tapered profile without utilizing such a specially tapered balloon. For instance, the stacked balloon variation of our system may be utilized to create such a deployed structure. A stent may be deployed first using an inner, smaller diameter, balloon and then, after movement of the catheter or core guide member, re-formed using an outer, larger diameter balloon situated to expand only a portion of the stent, e.g., its proximal part. Our restraining sleeve may also be used to prevent (or to control) the expansion of some portion of the larger balloon for precise control of the resulting stent profile. Alternatively, the balloon structure shown in FIG. 20D may be used to produce a tapered stent by simply placing the stent over both sections of the balloon (680) and, upon installation, inflate an inner balloon (682) at one end of the stent and an outer balloon (684) at the other end of the stent, e.g., perhaps the proximal part, to deploy a tapered stent.

Of course, our system may be constructed with radially stacked balloons, where at least one of the balloons do not have a constant diameter along their respective lengths. For instance, with a balloon stack of the type shown in FIG. 20B having a mounted stent, the innermost balloon (668) may be formed with a constant diameter (e.g., 2.5 mm), the middle balloon (666) may be formed with a smaller diameter at one end (e.g., 2.5 mm) and a larger inflated diameter at the other end (e.g., 3.0 mm), and outermost balloon may be formed with a smaller diameter at one end (e.g., 2.5 mm) and a larger inflated diameter at the other end (e.g., 3.5 mm), assuming substantially equal balloon lengths. For placement of a stent having a constant implanted diameter of 2.5 mm, the inner balloon (668) would be inflated. For a tapered stent having a 2.5 mm diameter at one end and a 3.0 mm diameter at the other end, the middle balloon (666) would be inflated alone. For a tapered stent having a 2.5 mm diameter at one end and a 3.5 mm diameter at the other end, the outermost balloon (664) would be inflated alone. For stents having constant, non-tapered, diameters of 3.0 mm or 3.5 mm, the middle balloon (666) or the outermost balloon (664) would be inflated alone, deflated, moved, and re-inflated to expand the stent to a common diameter, respectively. Such is the flexibility of our deployment system.

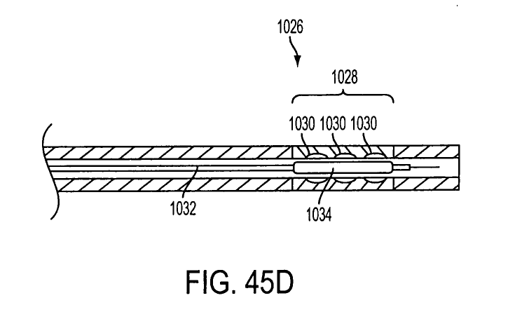

FIGS. 44A, 44B, and 44C depict a multi-balloon, radially stacked, balloon catheter comprising a set of atherotomes. As is the case with the other atherotome devices described herein, this device is inserted into an artery, placed at the site of a lesion, and expanded to cut or to break the plaque (when needed), for enhancing the treatment provided by a stent to be implanted later.

The balloon catheter section (990) shown in FIG. 44A is shown to have a set of three, radially nested balloons, an inner balloon (992), a middle balloon (994), and an outer balloon (996). Each of balloon (992, 994, 996) is shown to be adherent to a central catheter body (998). Although each of the balloons is shown to be directly adherent to the central catheter body (998), such a structure is not necessary or critical. The balloons may be in different configurations and, for instance, may adhere to each other. In any case, for the purposes of this example, each of the balloons is shown to have independent inflation of fluid access via fluid member (1000) to the outermost balloon (996), fluid member (1002) to the middle balloon (994), and fluid member (1004) to the inner balloon (992).

Outer balloon (996) is shown to have a set of atherotomes (1006). As is shown in FIG. 44B, a set of atherotomes (1006) is hidden within the depth of the material making up outer balloon (996). The upper edge (1008) of the atherotomes (1006) is sharp and is for the specific purposes of cutting the biological covering found on the noted lesions.

FIG. 44C shows the positions of atherotomes (1006) as the outer balloon (996) is either inflated or is subject to expansion by the inflation of one or more of the middle balloon (994) or the inner balloon (992). When the balloons are non-compliant in nature, the structure shown in 44A gives three precise blade or atherotomes (1006) expansion diameters or limits. The smallest expansion is that when the inner balloon (992) is expanded in isolation, the middle blade extension occurs when the middle balloon (994) is expanded alone or in combination with inner balloon (992). The largest extension occurs when the outer balloon (996) is expanded with or without the inflation of the inner balloon (992) and middle balloon (994). This variation provides a tool that is quite operationally flexible from the perspective of the physician user.

The balloons in this variation may further comprise elastic materials. In such a construction, the range of diameters available from this device are continuous rather than discrete, as would be the case with non-compliant balloons.

Tools for Use With the System

Stenotic Incision Tool

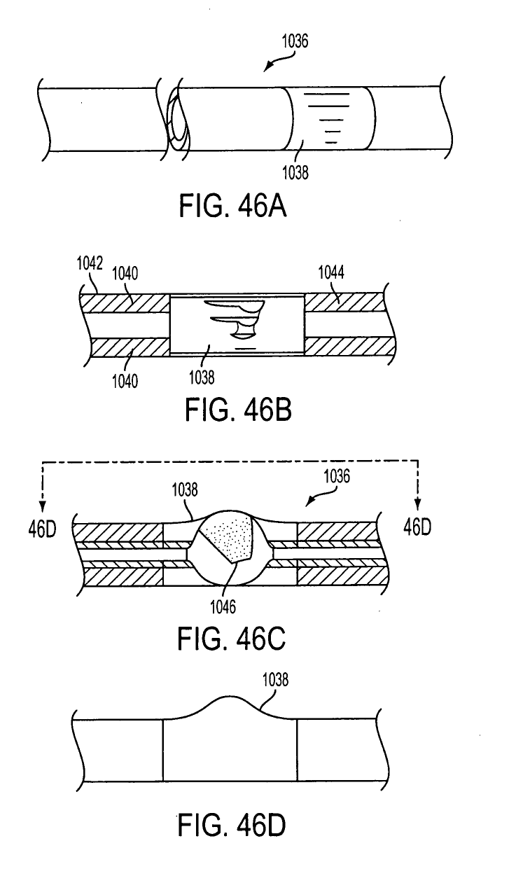

FIGS. 45A-45C show a stenotic incision tool suitable for cutting stenoses as may be found in a vascular lumen.

FIG. 45A shows a partial cutaway view of the tool (1010). Section (1012) includes the blades or atherotomes (1016), see, FIG. 45B. Optional slits (1014) are shown in the exterior of section (1012) to allow ease of passage of the atherotomes (1016) onto the stenoses to be cut. Alternatively, the slits may be formed by movement of the atherotomes (1016) as they are pushed radially by the inflated balloons.

FIG. 45B shows the atherotomes (1016). They are located in a position that is generally in line with the longitudinal axis of section (1012) and may be mounted in section (1012) in a variety of ways, e.g., mounted on an interior elastic membrane or pinned in a relatively solid elastomeric block of a type that when expanded by a balloon in the interior lumen (1018), will flex and allow the atherotomes to extend through the exterior wall of the section (1012).

FIG. 45C shows the introduction of a core guide member (1020) having a balloon (1022) that has been expanded within the lumen (1018) of the tool (1010) and has caused the atherotomes (1016) to extend beyond the perimeters of section (1012) and presumably into the stenoses. This tool may be an independent device mounted to a position control member (1024) and slid down over core guide member (1020). Alternatively, it may be combined with system components or devices having other functions such as the stenting delivery sleeve in a single device. Such a combination would be very useful in the cutting and stenting of hard stenoses since the combination tool need not be removed from the body between the cutting step and the stenting step.

FIG. 45D shows a variation of the stenotic cutting tool. The tool (1026) includes a cutting region (1028) containing a number of atherotomes (1030). These atherotomes (1030) are extended into a lesion by the inflation of a balloon catheter (1032) having perhaps a multi-layer balloon section (1034) and a distal balloon section (not shown). This balloon catheter allows better control of the depth of incision into a lesion. For instance, if the balloon section (1034) were to include three radially adjacent balloons, the balloons would be able to extend the atherotomes into the lesion at three different levels or diameters.

In addition, the one or more atherotome structures may be placed onto the outer layers of the balloons and a movable sleeve, as described elsewhere herein, may be placed over some portion of the atherotome structures to segregate the structures and to allow some of the atherotome structure to extend into the surrounding tissue and to prevent other portions from extending into other surrounding tissue.

Shape Control Member

Another tool useful in the described system is a shape control member for controllably limiting the expansion and shape of a removable expandable member. The tool, in essence, is a fabric caul, a preformed fabric shape or size that, when extended by a balloon, reverts to the expanded, selected shape or size. For instance, in the event that a user wished to implant a stent having a specific interior diameter, a fabric cylinder having that diameter would form the caul in the shape control member. Special formed shapes for a particular procedure or multiple diameter forms are all easily achievable using tools such as described here.

The shape control member may take the place of the constraining member discussed elsewhere, in some chosen circumstances or may be used in conjunction with the constraining member. For instance, the stent delivery sleeve discussed above may be used with a constraining member either to constrain a stent (upon the sleeve) while delivering another stent. Alternatively, the fabric caul may be used to constrain the portion of the balloon not beneath the length constraining sleeve to achieve a given diameter upon expansion. By coordinated use of the fabric caul to achieve a given diameter, the constraining sleeve to achieve a length, and the stent sleeve, one may achieve and select a stent length and diameter at the time of delivery.

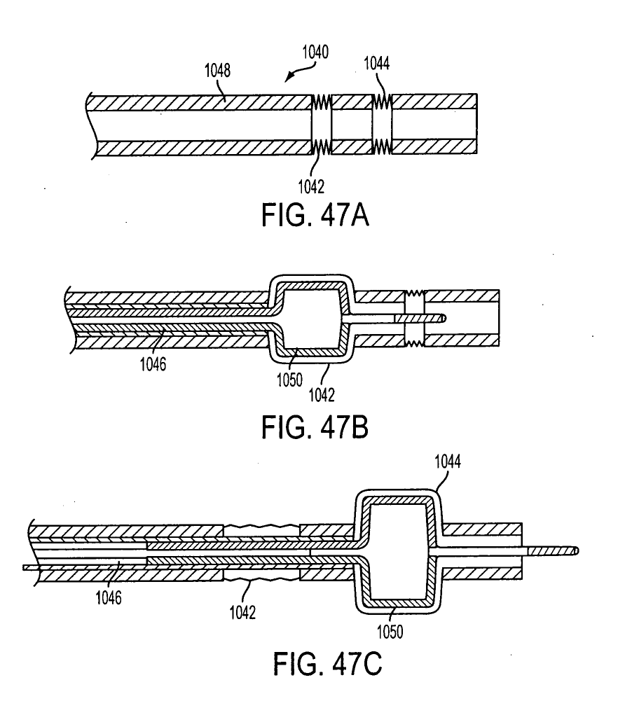

FIG. 46A shows the exterior of a shape control member (1036) in which the fabric caul (1038) has the expanded shape found in the exterior view shown in FIG. 46D. FIG. 46B shows a cross section of the device shown in FIG. 46A. The fabric caul (1038) may include one or more stiffeners (1040) located as struts to hold the ends of the caul apart, if needed, since the inner portion of the caul may comprise only a fabric, and to provide spacing between the proximal portion of the tool (1042) and the distal section (1044). Stiffeners (1040) are optional. The cauls (1038) may be supported by support members or longitudinal stiffeners (not shown here but are explained elsewhere) to provide a measure of specific form to the cauls (1038) as they are moved to a particular region of the body over, e.g., the core guide member.

FIGS. 46C shows a cross section of the tool (1036) with balloon (1046) expanded beneath a caul (1038) to provide the overall shape to the expanded caul (1038) as shown in FIG. 46D. In this example, the diameter of the caul is not constant. However, this tool permits many shaping functions otherwise quite difficult to perform in various anatomical passageways. For instance, in addition to the constant diameter and variable diameter variations of the device mentioned above used for the purpose of limiting the expansion of a balloon to a specific diameter, the shaping control member may be used to hold a vaso-occlusive material within an aneurysm as that material “sets.” Typical of such materials are biocompatible polymeric materials that precipitate from a solvent solution when introduced into the body, e.g., ethylene vinyl alcohol copolymer in DMSO, or biocompatible polymers formed in situ via reaction, e.g., various urethanes, cyanoacrylates, (C₁-C₆)hydroxyalkyl (C₁-C₆)alkacrylate (e.g., hydroxyethyl methacrylate), silicone pre-polymers, and the like. Those types of vaso-occlusive materials are described in U.S. Pat. Nos. 6,569,190, to Whalen II et al, the entirety of which is incorporated by reference. Additionally, other precipitative polymers are disclosed in U.S. Pat. No. 5,925,683, to Park, and its continuations and CIP's, specifically ethanolic—partially hydrolyzed polyvinyl acetate solutions. Other polymeric vaso-occlusive devices comprising extruded polymers such as polyacrylonitrile gels such as those described in U.S. patent application Publication No. US2002/0193813 A1. These materials may be placed and held as needed or desired using the noted device. These embolic materials often contain radio-opaque materials as well. Asymmetric angioplasty is attainable using such shape control members. Placement of stents into very wide-mouthed aneurysms may be achieved by a selection of a properly fitting fabric caul and its placement in a shape control member.

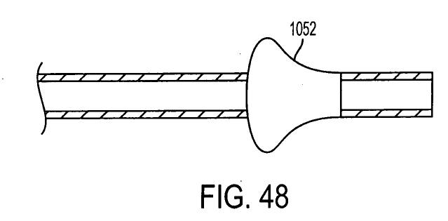

FIGS. 47A-47C show a shape control member (1040) having a pair of fabric cauls (1042, 1044). The expanded diameter of caul (1042) is smaller than the expanded diameter of caul (1044).

FIG. 47A shows the two cauls (1042, 1044) as they are to be slid over the core guide member (1046) in FIGS. 47B and 47C. By folding, crimping, or twisting cauls (1042, 1044), the proximal outer diameter of the adjacent position control member (1048) may be approached.

FIG. 47B shows the expansion of balloon (1050) into the more proximal caul (1042). As shown in FIG. 47B, the balloon in this variation completely fills the interior of caul (1042) and the caul expands to the desired diameter. Deflation of balloon (1050) and distal movement of the balloon to the more distal caul (1044), followed by inflation of balloon (1050) results in the production of a larger constant diameter form as was pre-selected in this variation. Fabric caul (1042) is obviously no longer inflated in FIG. 47C since the balloon (1050) has moved to another site.

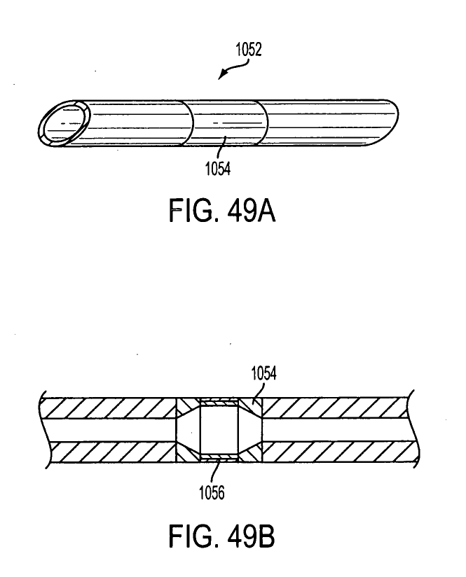

FIG. 48D shows a caul (1052) having a bell shape as might be suitable for reforming stents to a particular shape when needed. As should be apparent, the cauls used in this tool may be of any shape effective to do the task perceived.

The various cauls portrayed in FIGS. 46A to 48 each are shown to be in isolation with a support member of some kind defining the end of a separate fabric caul. However, the cauls need not necessarily be supported and separated in that way. For instance, the cauls may be joined, one to the other, without a supporting member intervening between them. Since the cauls are typically fabric, they may be directly joined to each other as is common in the medical fabric art.

In many instances, the tools described here may also be used with balloon catheters and with core guide members.

Drug Delivery Member

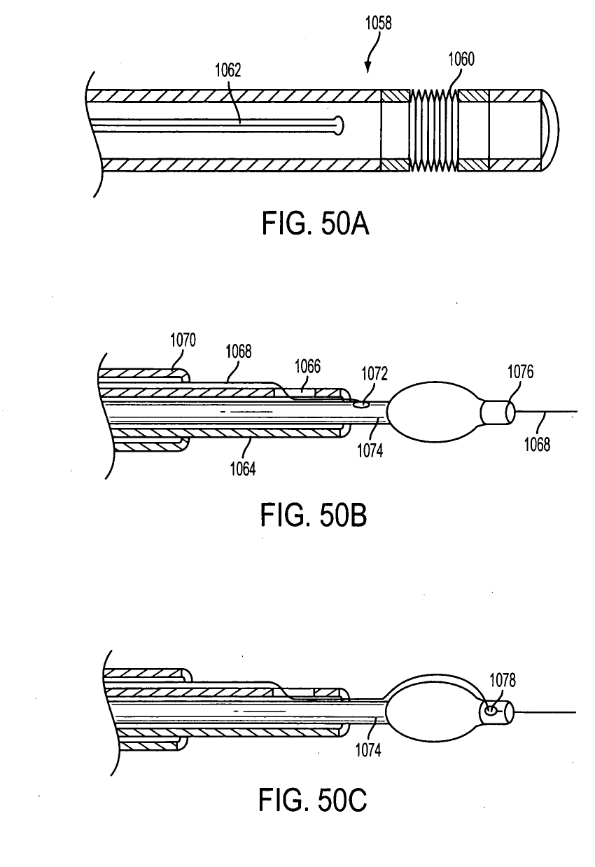

Another tool suitable for this system is one that delivers drugs or drug containing materials to the interior of a treatment site. FIG. 49A shows a partial exterior view of the drug delivery member (1052) with the drug containing section (1054) in the center of that section. FIG. 49B shows a cross sectional view of the drug delivery tool (1052). Drug delivery section (1054) in this variation is made up of rupturable drug containing membranes that, upon placement on an expandable or inflatable member or balloon, places the drug exterior to the delivery section (1054) and in contact with the tissue to be treated. Frangible membranes such as (1056) containing islands of fluid drugs are well known. Generically, a wide variety of drugs may be delivered using this tool. The drug delivery section may be designed such that contact with the outside lumen wall (a “squeeze”), by expansion of the balloon in the interior passageway, is necessary for initiation of the drug release. The drug delivery section may be designed such that contact with the outside lumen wall is not necessary for drug release; simple expansion being sufficient. This tool is of the type that a stent-graft comprising a stenting device of some kind (integral, discrete and inside the graft interior, discrete and supporting from outside the graft, interwoven with the graft material, etc.) and the graft (comprising the drug delivery carrier) may be deployed as a unit with this tool.

As was the case with the cauls above, the drug delivery section (or sections) may be separated by support members at the end of each drug delivery section or they need not be. Multiple drug delivery sections may be directly joined, if so desired, without supporting members between them.

Again, the tools described in this section may be used with all variations of the system. The wire exchange devices and concepts are also applicable to the other variations of the system and tools shown herein.

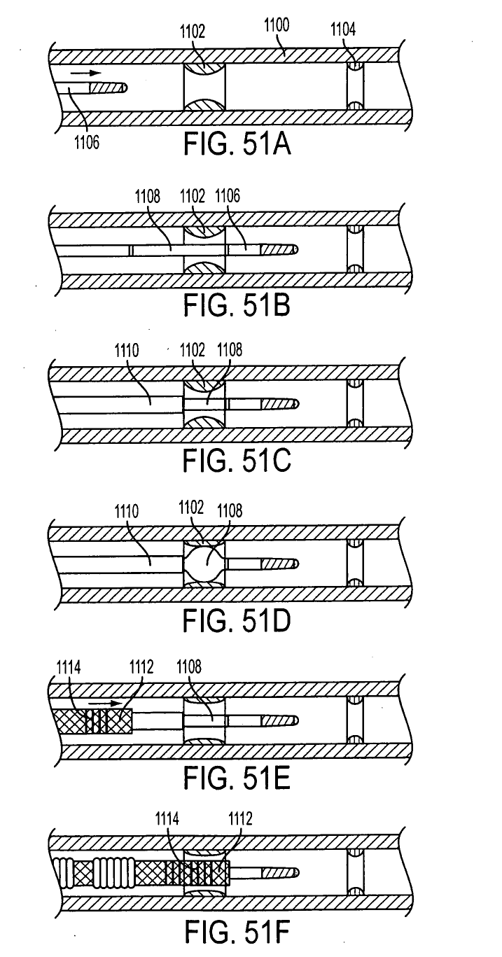

FIG. 50A shows a caul assembly (1058) that comprises a fabric caul (1060) used to provide (or, perhaps, to impose) a particular inflated shape to a balloon located interior to the caul or to provide a specific limited diameter to a balloon on a balloon catheter provided in the interior of the caul. Also included in caul assembly (1058) is a slot or slit (1062) through which a guidewire may extend to allow operation of some or all the components of the balloon catheter assembly as a guidewire exchange mechanism or perhaps better known as “rapid exchange.”

FIG. 50B shows the use of such a slot (1062) in one of the tools (1064) mentioned herein. The slot in tool (1064) appears as (1066). In the variation shown in FIG. 50B, guidewire (1068) leaves the distal end of guide catheter (1070), proceeds along the body of tool (1064) through slot (1066), and passes into an opening (1072) provided in balloon catheter (1074). Opening (1072) allows the guidewire to pass into a lumen in catheter (1074) and to pass to the distal end (1076) of the catheter. Guidewire (1068) may be seen extending distally from the distal opening of catheter (1076). This arrangement of passageway into the lumen of catheter (1074) is termed a “long” exchange.

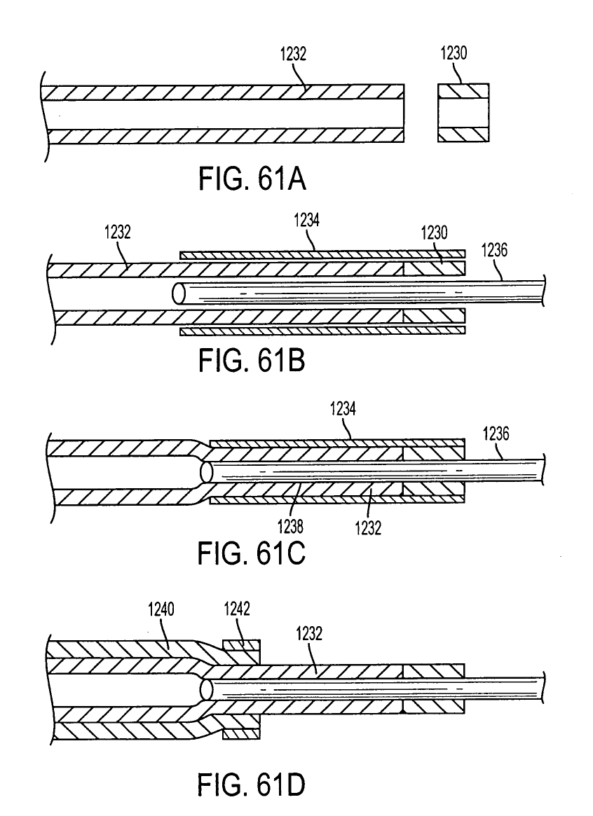

FIG. 50C shows the same general setup as found in FIG. 50B with the exception of the placement of the opening (1078) into the balloon catheter (1074). In this instance, the opening (1078) is distal of the balloon. This arrangement is called a “short” exchange. This arrangement and the long exchange arrangement may also be used with the other sleeves mentioned here, e.g., stent sleeve, cutting balloons, cutting tool, length constraining sleeve, etc. by use of a slot of the type shown in the FIGS. 62A, 62B, and 62C.

Other examples of catheter and guidewire exchange catheter systems are outlined in U.S. Pat. No. 6,692,465, to Kramer, the entirety of which is incorporated by reference. Any of the devices and methods shown there or discussed as background are applicable to this system with the exception, of course, that the portion of the constraining sleeve operating to constrain the balloon not involve a slit.

Each of the tools described in this section are equally useful with the core guide member variation of the system and the balloon catheter variation of the system.

Methods of Use

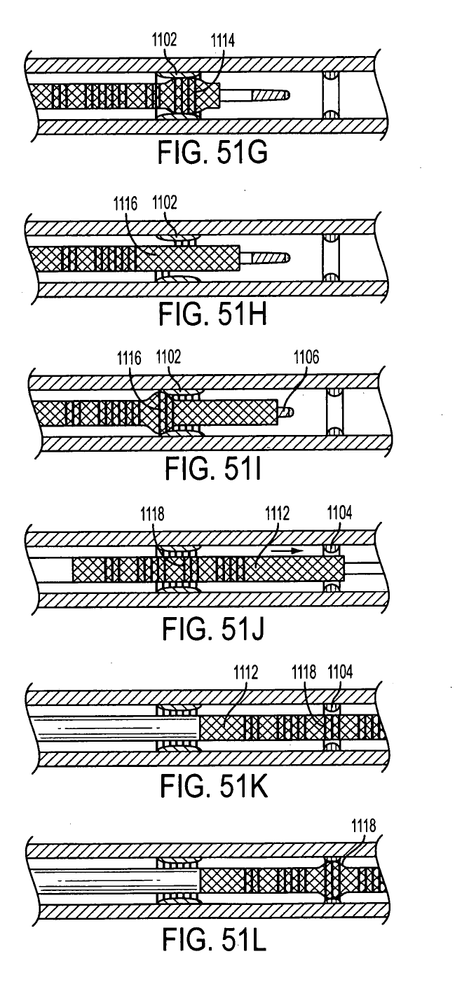

The steps shown in FIGS. 51A-51N show a complicated procedure that nevertheless, is significantly simplified from any corresponding procedure otherwise practiced using normal medical technology. The situation is this: artery (1100) has two regions of stenotic tissue, a proximal, larger stenosis (1102) and a more-distal stenosis (1104). The goal is to perform angioplasty upon both stenoses and to place stents upon those lesions. In FIG. 51A, the distal tip of core guide member (1106) approaches. For ease of understanding the physical manipulation of the system and its subcomponents, the severity of the depicted stenoses (depth of penetration and degree of blockage) are shown to be similar. Such is not normally the case.

In FIG. 51B, the balloon (1108) is placed such that the distal edge of the balloon corresponds to the distal edge of lesion (1102). In FIG. 51C, constraining member (1110) is introduced into a position such that its distal end corresponds to the proximal end of lesion (1102). This means that the distal end of the balloon corresponds to the distal end of the lesion and the distal end of the constraining member (1110) corresponds to the proximal end of the lesion. This exercise is for limiting the size of the balloon (1108).

FIG. 51D shows the expansion of balloon (1108) with the constraining member (1110) in place to perform an angioplasty upon lesion (1102), i.e., dilation or dilatation of the lesion. The balloon (1108) may be inflated with a variety of materials, fluids (including gases in rare instances), and liquids. Typically, though, the balloon in each of the variations disclosed herein will be inflated by liquid. A typical liquid is a biological saline solution, perhaps containing a biocompatible dye or contrast agent such as metrizamide, iopamidol, iothalamate sodium, iohexol, iodomide sodium, or meglumine.

In FIG. 51E, balloon (1108) has been deflated and the stent delivery sleeve (1112) containing a number of stents (1114) approaches.

In FIG. 5IF, stent delivery sleeve (712) has been placed so that stent (1114) is in proper position for implantation. FIG. 51G shows the result of inflating balloon within stent (1114) for placement on stenosis (1102). In this instance, the physician has determined that an additional stent would be desirable and chooses to position stent (1116) over the proximal end of lesion (1102) as shown in FIG. 51H. In FIG. 511, the balloon longitudinal size has been adjusted to a smaller value and the balloon then expanded to place stent (1116) on the proximal end of lesion (1102). Note that the core guide member (1106) has been moved proximally with respect to the constraint member (1110) (to allow proper placement of the balloon at the selected site) and to shorten the effective length of the balloon. In FIG. 51J, the physician moves the core guide member and the stent delivery braid (1112) distally down to smaller lesion (1104) and chooses not to perform a pre-dilatation step there, i.e., the physician opts to perform direct stenting. In FIG. 51K, the chosen stent (1118) has been positioned over lesion (1104) and in FIG. 51L, the balloon has been expanded to place stent (1118) on the smaller lesion without pre-dilatation.

FIG. 51M shows the deflation of the balloon (1118).

FIG. 5IN shows the ultimate placement of the stents upon lesions (1102, 1104) and the withdrawal of the system and its core guide member (1106). A similar procedure may have been conducted with a guidewire and balloon catheter replacing the core guide member.

A common procedure considered to be minimally invasive to the patient and fairly effective in solving problems associated with small necked neurovascular aneurysms, is the placement of embolism-forming materials or structures in the aneurysm and in some instances placing various types of stenting devices over the mouth of the aneurysm. The embolic material may be any of a number different types. Embolic materials such as the precipitative and reactive polymeric materials discussed above and solid materials such as micro-coils (described in U.S. Pat. No. 5,122,136, to Guglielmi, and its related U.S. patents, incorporated by reference) delivered using an electrolytic joint or by other methods are all suitable for placement with the described device. Other polymeric vaso-occlusive devices comprising extruded polymers such as polyacrylonitrile gels such as those described in U.S. patent application Publication No. US2002/0193813 A1. These materials may be placed and held as needed or desired using the noted device. In any case, it is occasionally a challenge to maintain the presence of the embolic materials wholly within the aneurysm. Loss of even a small amount of embolic material in the neurovasculature can be catastrophic.

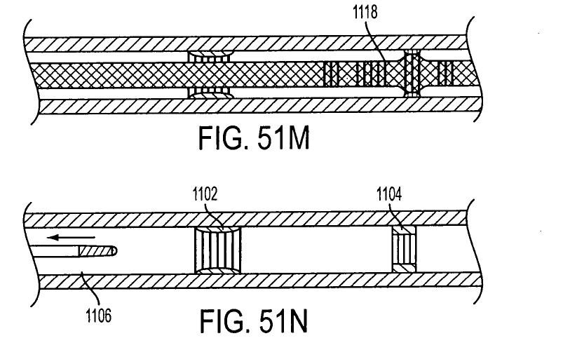

FIGS. 52A and 52B show, in summary fashion, placement of a stenting device (1120) over the mouth an aneurysm (1122) containing some type of an embolic material (1124). As has been the case with other procedures shown here, the stenting device (1170) is suitably placed at the mouth of the aneurysm (1122), the balloon (1126) is adjusted for size using the constraining member (1128), and the balloon (1126) is then inflated to place the stent (1120) in proper alignment with the aneurysm mouth. FIG. 52B shows such alignment and the removal of the system (1130).

Our catheter-based system may be used in several different ways to deploy such a stent: a.) the balloon catheter in conjunction with the shape control tool (having a fabric caul), b.) a balloon catheter having several small balloons inflated in tandem, and c.) the expanded balloon catheter beneath a simple constraining member.

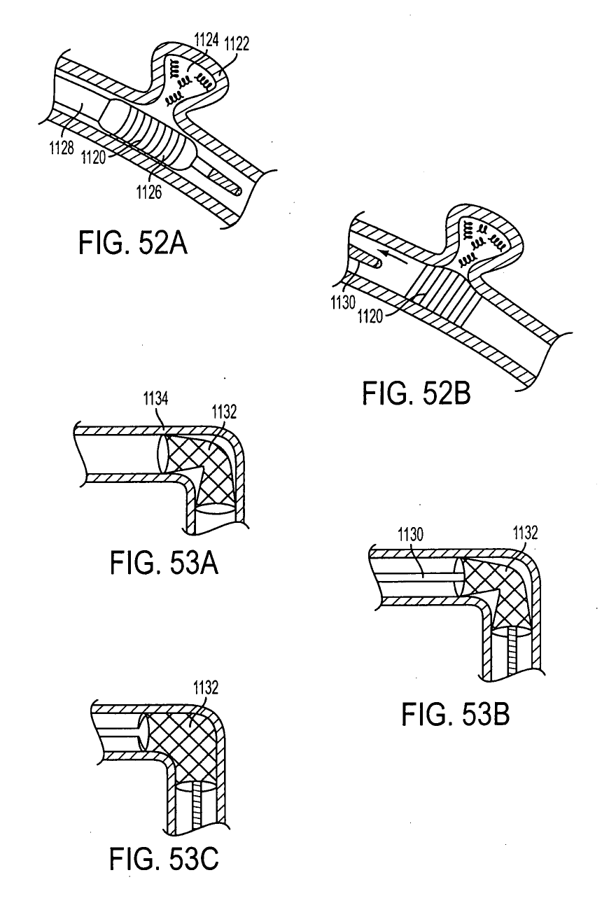

The system described here, because of its size, physical flexibility, and operational flexibility, is able to correct problems created by the use of other less facile devices. For instance, FIGS. 53A-53C shows a procedure for correcting a kink in a stent (1132) found in the elbow of an artery (1134), or in other “sick” or stenosed arteries with calcified sections. The extent of the problem is exaggerated in the drawings for the sake of explaining a solution of the problem. Placement of stents in sharply bending portions of the vasculature presents significant challenges in procedures employing normal balloon expandable stents. Much of the problem stems from the relative lateral inflexibility of many balloons used in cardiovascular medicine to expand the stent.

FIG. 53B shows the placement of the system described here (1130) through the central passageway of the bent stent. Adjustment of the longitudinal balloon size to a small length and repeated inflation, deflation, incremental movement of the balloon toward the kink, inflation, deflation, incremental movement of the balloon, etc., allows the user to reform stent (1132) in the fashion shown in FIG. 53C.

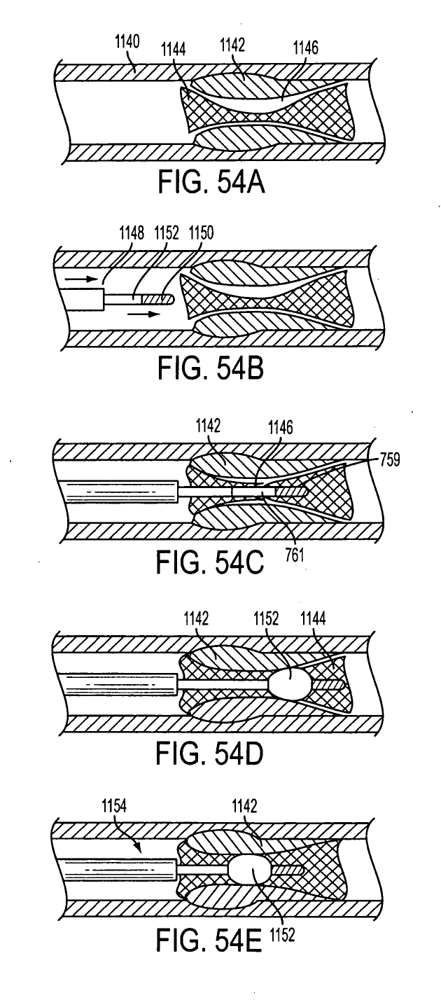

FIGS. 54A-54J depicts, in cross section, a procedure for re-forming a stent using the described system where the stent has been placed on lesion, perhaps calcified in a reasonably straight arterial section. Ancillary steps for dilation with stent in place are also shown.

In FIG. 54A may be seen an artery (1140) having a fairly significant lesion (1142) that, because of its size and depth, may be fairly hard, that is to say the exterior may be calcified. The stent (1144) is shown in FIG. 54A to have a kink (1146) in a more distal position in lesion (1142). Since access to lesion (1142) will be proximally (from the left in FIG. 54A) the kink (1146) is very hard to reach because of its more remote location in the lesion.

FIG. 54B shows the approach of the described device (1148). Core guide member (1150) with balloon (1152) may also be seen approaching the stent.

In FIG. 54C, the core guide member (1150) has penetrated the stent and the balloon (1152) has been placed in the vicinity of the stent kink (1146). Because of the exceptionally small diameter of the core guide member (1152), repair of this stent anomaly is possible and it results in additional opening of the vascular passageway through lesion (1142).

FIG. 54D shows inflation of balloon (1152) to remove the kink and permit the stent (1144) to better conform to the interior shape of lesion (1142). The low profile and overall functional flexibility of the disclosed device permits the kink in an otherwise useful stent to be repaired.

In addition, the device (1154) may be used to perform additional dilation on lesion (1142). After the balloon (1152) has been deflated in FIG. 54D, the balloon (1152) may be moved proximally and re-inflated as shown in FIG. 54E to dilate another portion of lesion (1142).

Similarly, FIG. 54F shows still another dilation step where the balloon (1152) is simply re-inflated in another portion of lesion (1142) to dilate yet another more proximal portion.

FIG. 54G shows deflation of balloon (1152) and the approximate profile of both the stent (1144) and the lesion (1142) after the dilation efforts described above.

FIGS. 54H and FIG. 54I show the use of constraining member (1160) in conjunction with balloon (1152) to limit the axial length of that balloon (1152) and dilation of the lesion (1142). The proximal end of the lesion (1142) and stent (1144) have been significantly improved by this procedure.

FIG. 54J shows the removal of the device from the region of lesion (1142) and shows results of such treatment where the kink (1146) in stent (1144) is gone, the conformance of the stent to the vasculature is very good, and the passageway through lesion (1142) is significantly wider.

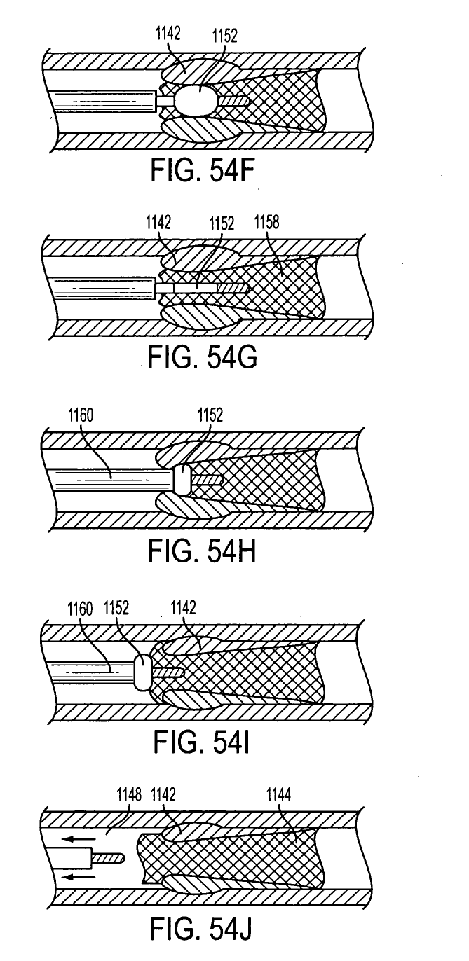

FIGS. 55A-55E, in turn, show the relative facility of placing one or more stenting implants in an arterial bend using our described system, even where the lesion is found in a difficult region of the bend.

FIG. 55A shows a lesion (1170) in the bend of an artery (1172). The lesion (1170) has a narrow neck, is in the bend of the artery, and is otherwise fairly difficult to access and treat.

FIG. 55B shows the introduction of the core guide member (1174) and its passage through the opening of lesion (1170). The incorporated balloon (1180) is shown positioned with the distal end (1182) of the balloon (1180) slightly distal of the lesion (1170). In this instance, prior angioplasty is either not shown or is not considered desirable or, at least, appropriate for the procedure. Stent sleeve (1176) and stent (1178) are also shown in the Figure.

FIG. 55C shows placement of the stent delivery sleeve (1176) with an included stent (1178) over lesion (1170). FIG. 55C shows the placement and location of the balloon (1180) within the stent delivery sleeve during the step of placement of stent (1178).

FIG. 55D1 shows the expansion of the balloon (1180) beneath the stent (1178). Note that the length of the balloon (1180) shown in FIG. 55D2 is similar to the length of the stent (1178) on stent delivery sleeve (1176). This adjustment is achieved by movement of the constraining member (not shown in these Figures) discussed above or by inflation of several balloons longitudinally. Inflation of balloon (1180) provides a large measure of complete stent deployment. FIG. 55D3 shows that the stent (1178) is not completely conformed at position (1190) to the vessel wall. FIGS. 55D2, 55D3, 55E2, and 55E3 are portrayals of the component of interest with all blocking components removed for clarity.

FIG. 55E2 shows withdrawal of stent delivery sleeve (1176) to permit direct contact between the balloon (1182) and stent (1178). That the stent (1178) may not have been in absolute conformance to the shape of lesion (1170) may now be ameliorated. FIG. 55E2 shows the expansion of the balloon with its adjusted and shorter longitudinal length. This allows “touching up” of the expansion and placement of the stent and its ultimate conformation with the surface of the lesion. The conformance is shown in FIG. 55E3. Conformance is especially needed in the distal and proximal ends of the stent, areas of the stent most likely not to contact the vessel wall effectively. Restenosis is particularly a problem at the distal and proximal portions of drug-eluting stents and more generally when the stents do not contact the wall well or do not contact the wall at all. Good vessel apposition in all areas is of prime importance. FIG. 55F shows withdrawal of core guide member (1174) and the resulting placement of stent (1178) over lesion (1170).

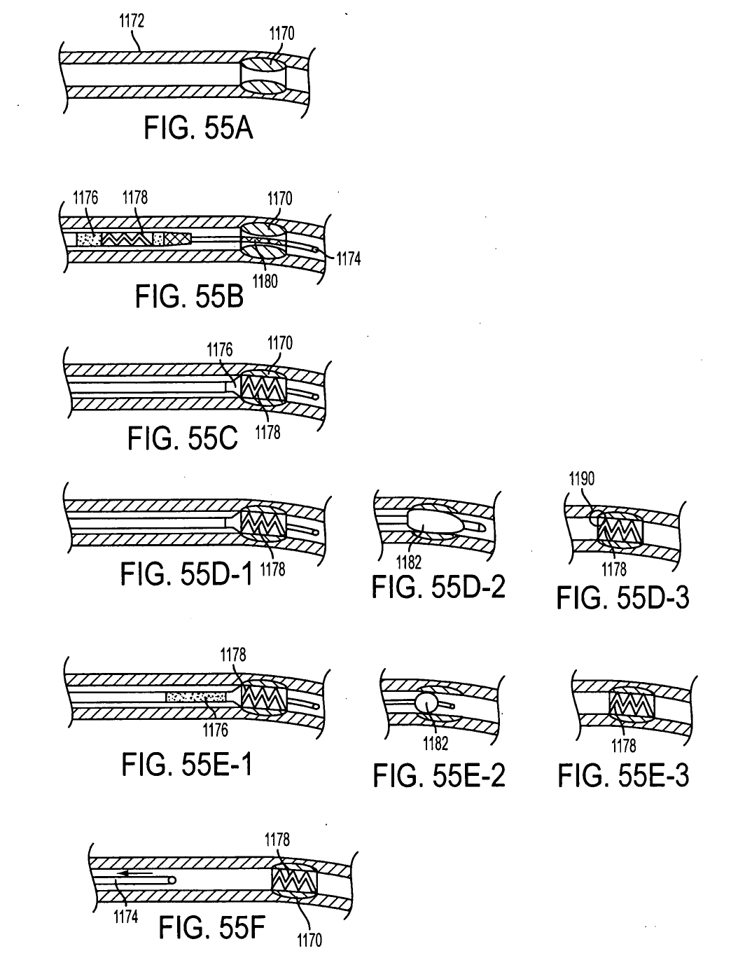

FIGS. 56A-56E show the use of the core guide member variation shown in FIGS. 11A and 11B, discussed above, having a thin profile, distal balloon member, and proximal section having a number of balloon members in a stack.

FIG. 56A shows a vascular artery wall (1190) and a lesion (1192) situated in that wall. Lesion (1192) has a fairly narrow passageway through its middle and consequently is a substantial blockage to the flow of blood. Approaching lesion (1192) is variation (1194) having a narrow profile distal balloon section (1196) a multi balloon section (1198) located more proximally and constraining member (1200). Because the distal tip section (1196) incorporates such a narrow profile, it readily passes into the narrow passageway of lesion (1 192) as is shown in FIG. 56B. FIG. 56C shows inflation of balloon (1196) in the narrow distal balloon section and the consequent reforming of dilation of the lesion (1192).

FIG. 56D shows the distal movement of narrow distal balloon section (1196) past lesion (1192), the introduction of multi layer balloon section (1198) and its subsequent inflation. FIG. 56E shows the retraction of device (1194) and the results of dilating lesion (1192). The marked difference between the profile of the distal balloon section (1196) and the more proximal stacked balloon section (1198) allowed this treatment to be effective in the way shown.

As mentioned above earlier, the delivery system may be used to perform direct stenting with or without a stent delivery sleeve.

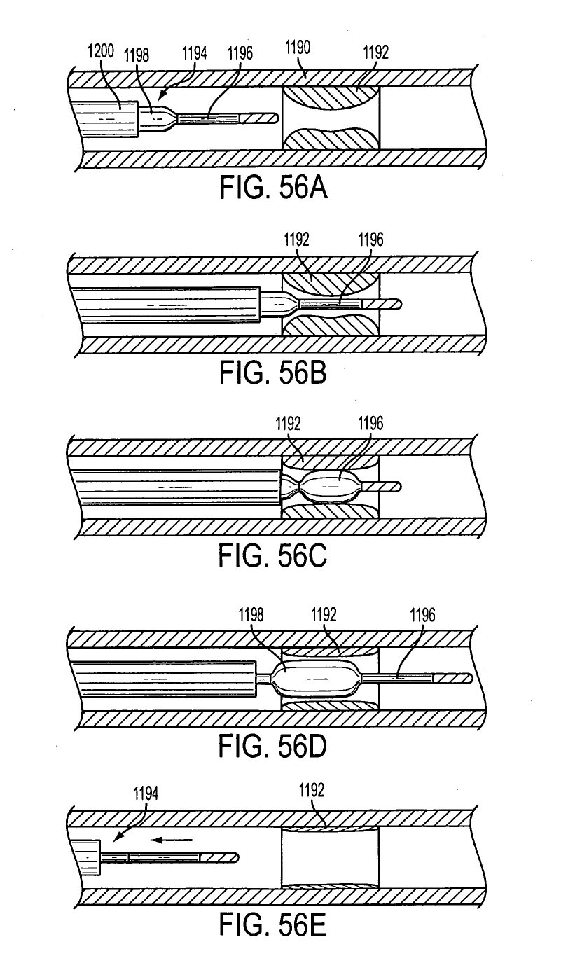

FIG. 57A shows an artery (1202) with lesion (1200). FIG. 57B shows the introduction of a core guide member (1204) with balloon (1206) and stent (1208) situated directly over, in direct contact with, balloon (1206).

FIG. 57C shows placement of stent (1208) in a position for expansion.

FIG. 57D shows expansion of stent (1208) with balloon (1206). FIG. 57E shows placement of the stent (1208) after deployment and withdrawal of the core guide member (1204).

Valving for Inflation of Multi-Balloon Catheters and Related Procedures

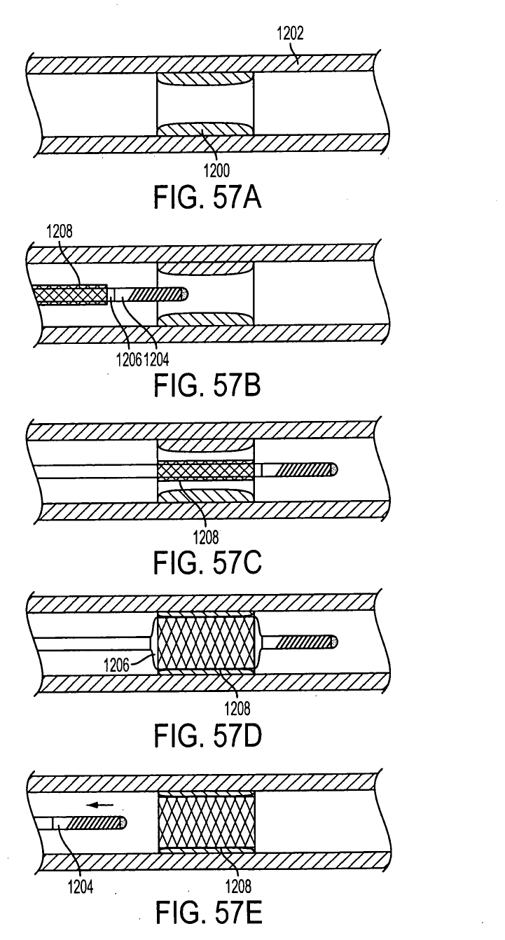

FIGS. 58A, 58B, 59A, and 59B show the versatility of the multi-balloon system in placement of a stent. The depiction provided in these figures is somewhat exaggerated in proportion and schematic in nature to illustrate the operational point described here. FIG. 58B may be also considered a description of a single step process for deploying a stent. FIGS. 59A and 59B may be considered a multi-step procedure for deploying a stent. In any case, one point to understand in this description is the potential for relative ease of operation in changing the size of a stent after deployment “on the fly” without the necessity of removing the balloon catheter.

FIG. 58A shows a balloon catheter assembly (1210) having a section upon which one or more stents (1212) are placed. The balloons section includes an inner balloon (1214) with an independent inflation feed line (1216), a middle balloon (1218) having an independent feed line (1220), and an outer balloon (1222) with an independent feed line (1224). Each of the three feed lines is shown to be connected to a valve located proximally near the user. Inflation of the inner balloon (1224) or “S” balloon, is controlled by a valve (1230). The inflation of the middle balloon, or “M” balloon is controlled by valve (1232) and inflation of the outer balloon (1222) is controlled by valve (1234). The valves are shown to be fed by a common supply line (1236) as would be the normal case in such instances.

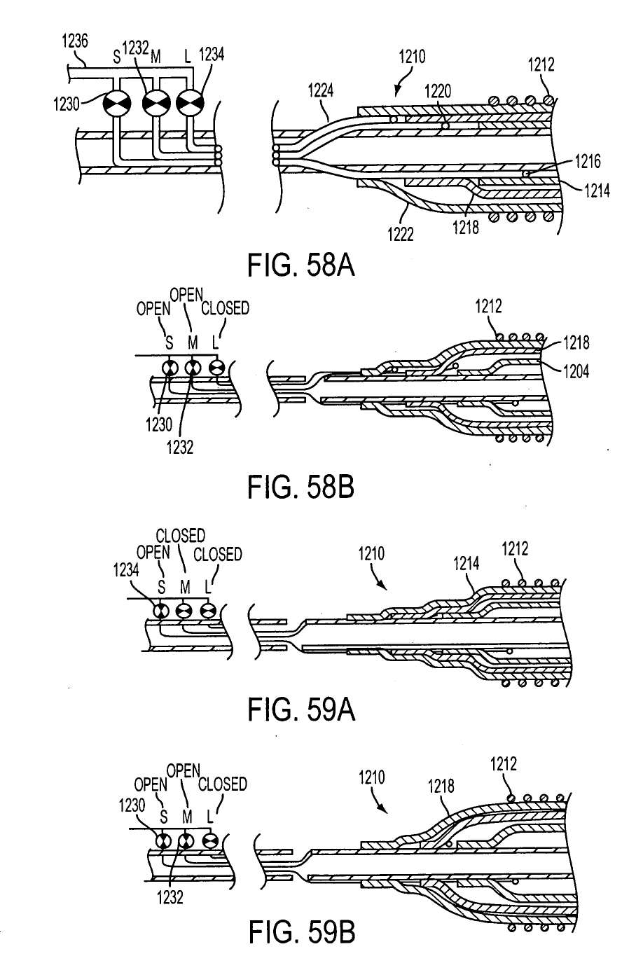

Although the fluid supplies to the various balloons are shown to be controlled by independent valves, the valves may be ganged or arranged in a single control device, the function of which will be described with respect to FIG. 60 below, for safe and easy use by the user-physician. It should be noted that the arrangement shown in FIG. 58A is one in which during deflation of the balloons, the inflation fluids would flow back through each of the valves.

FIG. 58B shows the step of simultaneously inflating inner balloon (1214) and middle balloon (1218). In this instance, balloon (1214) is inflated by opening valve (1230) and the middle balloon (1218) is inflated by opening valve (1232). Because two flow lumen are opened to fill what may be viewed as a single volume—the volume interior to middle balloon (1218), the inflation is faster than would be the case if a single lumen were used by opening only valve (1232).

This implantation step where stent (1212) is implanted upon a lesion, may be either a direct stenting step wherein the lesion is stented without a prior dilation step or maybe a simple implantation step performed after dilation.

As noted elsewhere many times here, the devices shown here, particularly those utilizing multiple stacked balloons, is operationally quite flexible in that the physician-user is able to assess and change the treatment during the course of stent implantation. FIG. 59A and FIG. 59B show a two-step method in which a stent (1212) is fitted to a lesion during a procedure.

FIG. 59A shows the inflation of inner balloon (1214) by opening valve (1230). If, after inflation of that inner balloon, the physician observes the conformance of the stent to the lesion and to the vascular wall could be improved by increasing the diameter of the stent, the physician has the available choice of inflating the middle balloon (1218) to enhance that conformance.

In inflating the middle balloon (1218), first, the fluid used to inflate inner balloon (1204) is held in the inner balloon by (at least) maintaining the valve (1230) in a closed position. Inflating the middle balloon (1218) is the next operation. Opening valve (1232) will allow passage of additional inflation fluid to pass into and to inflate middle balloon (1218) thereby expanding stent (1212). Maintaining the volume and pressure in the inner balloon (1214) before and during the step of inflating the middle balloon is seen as necessary to prevent serious problems such as movement or shifting of the balloon catheter (1210), movement or shifting of the stent, and—most serious—release of the stent. Even transient changes in the volume and pressure of the inner balloon (1214) are seen to be potential problems.

After inflation of the balloons in the steps shown variously in FIGS. 58B or 59B, the balloons are deflated by removal of contained fluids and the catheters are removed from the body leaving an implanted stent (1212) in each case.

FIG. 60 shows a schematic outline of the operation of a simple valve device or apparatus controlled by the physician-user in operating a multi-balloon catheter or core guide member as described here. The device allows the user to choose an effective and specific balloon diameter (when the balloons are non-compliant) while simply taking care of a number of advantages of the device and its design without additional input by the user. Other control schemes may be needed for expansion of stacked compliant balloons to previously chosen diameters. In any case, use of a simple expansion device is desirable both to alleviate operational safety concerns and to simulate as closely as possible prior practices. Most prior valve designs involved a single, simple inflator/deflator. This was so because those prior devices inflated but a single balloon. Where the multiple balloons often utilized in our systems require that the user actually manipulate input to several balloons (even while maintaining or controlling pressure in other balloons), there is no reason that the supply device or control device be complicated. Here, the inflation fluid is provided from a supply (1300). The fluid is provided to the balloons under pressure. Shown in FIG. 60 is a rotary valve (1304) that, in operation, acts as a flow controller and as an on/off valve. Valve (1302) is simply an on-off valve allowing a user to make a connection to the supply and, in overall operation of the system, may be considered to be optional. The multi-valve set-up used to direct fluid to one or more balloons may be a rotary valve as is depicted in FIG. 60 or it may be a set of single valves. Rotary valve (1304) is shown to have five positions when used in conjunction with a balloon catheter having three balloons. In this variation, switch “position 1” is shown to be “closed”. It is a safety position where no fluid flows to the balloon catheter. Switch “position 2” of rotary valve switch (1304) is shown to direct fluid through a check valve or one-way valve (1306) to the conduit that eventually reaches the inner balloon. One-way valves, sometimes known as check valves or stop valves, are valves allowing flow of fluids in but a single direction.

Similarly, switch “Position 3” allows passage of inflation fluid through a check valve (1308) ending ultimately in the middle balloon. Switch “Position 2” and “Position 3” are connected through a one-way valve (1310). In the event that this device is used with a stacked, multi-balloon, balloon catheter, the check valve (1310) allows fluid introduced through switch “Position 3” also to flow into the lines passing to the inner balloon. This means that when switch “Position 3” is chosen, both the inner balloon and the middle balloon are filled through their respective check valves (1306, 1308). Similarly, switch “Position 4” directs fluid to the outer balloon through check valve (1312) and through check valve (1314) to smaller balloons. Again, for the user to initially choose switch “Position 4” as the switch position for filling the balloons on the balloon catheter, the series of one-way valves shown here will cause the filling up all three balloons. Switch “Position 5” is another position where no fluid is directed to any of the balloons from fluid supply.

Deflation of the balloons is less specific in many cases—the physician normally would not elect to deflate the balloons one at a time—and consequently this variation does not provide such an ability, although it would be an easy matter to do so. When the rotary valve (1304) is in a closed position, deflation valve (1316) may be opened and the inflation fluids in the respective balloons will flow through check valves (1318), through valve (1316), and out for disposal. The deflation one-way valves (1318) prevent fluid from one balloon passing into another.

The interlock (1319) between deflation valve (1316) and rotary valve (1304) is designed so that when fluid flows into the balloons through valve (1304), the deflation valve (1316) remains closed. The rotary valve (1304) then directs fluid to the selected balloon or balloons. Similarly, when deflation valve (1316) is open, inflation valve or rotary valve (1304) will remain closed, or at least will not allow introduction of inflation fluid to the balloons.

It is within the scope of the disclosure here that the various selector variations may be used to select various physical deployed parameters of the deployed devices, e.g., deployed stent parameters such as stent diameter and deployed stent length (or “effective length” where multiple linked or unlinked stents are deployed forming a single stenting structure) or vena cava filters or the like, by selection of a selection value on the inflation valve. Similarly, the selector variations may be used to select physical parameters on devices that are not implanted, but instead are extended, e.g., such as artherotomes (whether mounted on a movable sleeve or mounted in a balloon). These selectors may be used with the generic classes of devices that are variously deployable, mountable, and installed in balloons (or other expandable devices).

It should be further apparent that the valve controls may easily be arranged to cause any configuration of balloon expansion desirable or appropriate for a perceived needed procedure. For instance, the valve control may be configured in such a way that rotating it causes a concomitant increase in the deployed stent diameter (as discussed above). The control may also be configured to increase the length of the deployed stents upon rotation of the control. Similarly, a designer may find it desirable to place a more than one such rotary valve in a convenient arrangement, e.g., concentric, side-by-side, one up—one down, etc. to permit easy selection of stent diameter and length and configuration (perhaps, tapered or hour glass shaped) with a single control box. Digital control of balloon inflation for such procedures is contemplated variously to optimize balloon filling rates and provide feedback information relating to the resulting stent deployment configuration and also to provide safeguards against inadvertent errors.

Example of Building a Stacked Balloon Catheter

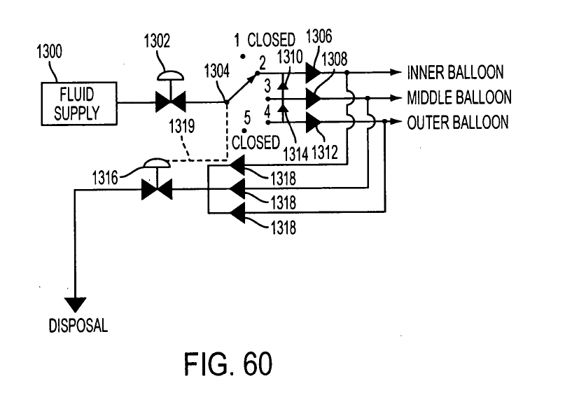

FIGS. 61A-61J schematically depicts a method for producing a catheter section of a balloon catheter having multiple layers such as the catheter shown in FIG. 20B.

FIG. 61A shows the step of placing the distal tip (1230) to the polymeric inner tubing member or catheter body (1232). Distal tip (1230) is typically a fairly soft material, e.g., perhaps of an elastomer, suitable for smooth progression through the vasculature. Desirably, the tip is of a material that is polymeric, miscible with, and certainly will adhere to the material making up the catheter body (1232). It is made to adhere to the catheter body (1232) in FIG. 61B. The inner tubing member is also known as the “inner lumen.” The catheter body in this example may comprise a composite of a PEBAX material with polyethylene (e.g., the composite TRILAYER tubing, PEBAX and polyethylene, sold by Putnam Plastics).

FIG. 61B illustrates the step of narrowing the distal region of the catheter body (1232) and causing the distal tip (1230) to adhere to catheter body (1232) by placing a sleeve (1234) of a material that shrinks upon application of heat, i.e., a “heat shrink” material, over the distal end of the catheter body (1232). A mandrel (1236), in this case a steel rod or wire having an outside diameter (OD) of about 15 mils. (0.015″), is also inserted into the interior of the catheter body (1232), the inner lumen. The mandrel may be treated to allow ease of removal, e.g., by coating with a lubricious or slippery polymer such as Teflon or poly(para-xylylene), e.g., Parylene. The construction concept used in this step is: the temperature used to shrink the heat shrink sleeve (1234) will also cause the polymers in both the distal tip (1230) and the catheter body (1232) to flow in some small amount and to controllably be squeezed down to the size of the mandrel. The result of the FIG. 61B heat shrinking is portrayed in FIG. 61C.

The inside diameter (1238) of the distal end of catheter body (1232) now matches the exterior diameter or OD of mandrel (1236). The heat shrink sleeve (1234) is removed and discarded.

FIG. 61D shows the initial placement of the first or innermost balloon (1240) upon the catheter body (1232). A heat shrink band (1242) is shown on the distal end of inner balloon (1240). As was the case with heat shrink tubing (1234), the heat shrink band (1242) may be discarded after a completion of its heat shrink step.

FIG. 61E shows the placement of the proximal end of inner balloon (1240). One of the inflation fluid fill lines is first positioned. A mandrel wire having a diameter of about six mils (1244) is inserted into a section of, e.g., Nylon 12 tubing (1246) and placed along catheter body (1232). The tubing (1246) is positioned so that it extends inside the inflation volume of inner balloon (1240). A band of heat shrink tubing (1248) is placed as shown so that when heat shrink tubing (1248) is heated, it pulls down the proximal end of inner balloon (1240) capturing the small tubing (1246) (also known as a “liner”) with its included mandrel. Upon heating heat shrink tubing (1248), the inner balloon (1240) will be sealed. After the heat shrink step is completed, the heat shrink band (1248) is removed and discarded.

Returning to the distal end of the assembly, FIG. 61F shows the placement of the distal end of middle balloon (1250). As is done in the other steps, heat shrink band (1252) is applied to the region of middle balloon (1250) that is to adhere to catheter body (1232). Again, after completion of the heat shrink step with band (1252), that heat shrink band is removed.

Returning to the more proximal end, FIG. 61G shows the placement of the proximal end of middle balloon (1250) and its positioning with a heat shrink tubing band (1254). As was the case with a feed line to the inner balloon (1240), the fluid feed line (1256) having an included mandrel (1258) is secured in place as shown in step seven of FIG. 61. When the respective wire mandrels (1244, 1258) are removed from their respective tubing members, those tubing members (1246, 1256) will be open to the interior of their respective balloons.

As is done in the other steps, after completion of the heat shrink step, heat shrink band (1254) is removed.

Returning to the more distal portion of the section, FIG. 61H shows the placement of the outer balloon (1258) with a heat shrink band (1260) positioned to cause the balloon distal collar to adhere to the polymeric tubing making up the catheter body (1232). The heat shrink band (1260) is again removed after use.

Returning to the more proximal portion of the catheter section, FIG. 61I shows the placement of proximal end of outer balloon (1258) and the heat shrink band (1260) over catheter body (1232) and another small fluid feed tube (1262) also including a mandrel (1264).

In FIG. 61J may be seen the final section. The more proximal and the more distal portions of the catheter section are placed in juxtaposition. An adherent polymeric covering (e.g., a polyester based shrink tubing) (1266) has been introduced over the proximal end of the balloon stack. This covering is both for protection and for the purpose of holding the three fluid feed tubing members to the catheter body (1232) at positions more proximal than those shown in the drawing. The various wire mandrels (1244, 1258, 1264) may be removed once that protective layer (1266) has been situated. The inner lumen mandrel (1236) has been removed in step 10. The various fluid feed lines may be attached to the controller valves as are shown, for instance, in FIG. 60.

The various balloons shown in this example are generally quite small. The target outer, folded diameter is 0.032″ or 32 mils. The feed lines (1246, 1260, 1262) are similarly shown to be quite small, e.g., having an ID of 7 mils and an OD of 9 mils. We have found that by joining these feed lines to larger tubing, e.g., with 11 mil ID and 14 mil OD, located proximally, that the flow rates to and from the balloons are quite good and the resulting inflation rates are comparable to those found in current cardiac balloon catheters. We have found that using high density polyethylene (HDPE) as the feed tube facilitates fluid removal.

It should be apparent that the diameters of the various feedlines may be selected as desired for the task at hand. The various feed lines may be of different sizes in the same device, for instance. The diameters may be of varying sizes from one end to the other, etc.

Rapid Exchange Mechanism

FIGS. 62A, 62B, and 62C show a balloon catheter system employing a rapid exchange mechanism and employing the constraining member discussed elsewhere herein. We discuss the combination using a constraining member so that a complete understanding of how one of our sleeves (e.g., the stent delivery sleeve or other sleeve-containing components that may be used with a balloon or other expandable member) may be designed for use with an exchange wire. This discussion is intended to teach the reader how to incorporate cooperating features into any of the sleeve-bearing components we describe here, but particularly the length constraining sleeve.

FIG. 62A shows a two-layer balloon catheter (1300). This particular exemplified balloon catheter (1300) includes an outer balloon member (1302) and an inner balloon member (1304). As is the case with these designs, each is radially adjacent to the other and adhere to the center core tubing (1306) of the catheter (1300). Other variations of the multi-balloon catheter as described elsewhere herein are also applicable in this arrangement. Also shown in this FIG. 62A is a constraining sleeve (1308). Constraining sleeve (1308) is built and used in the manner otherwise described here. This system, however, includes a rapid exchange wire (1310). This wire (1310) proceeds from the proximal end of the balloon catheter (1300), through a slotted opening (1312) in constraining member (1308), and through an opening (1314) in the wall of balloon catheter (1300). The design and use of rapid exchange wires as guidewires is well-known in catheter technology, although many exchange systems simply utilize conventional guidewires.

The use of a constraining member in this instance is unique. The constraining member (1308) includes a slot (1312) that is particularly adapted to allow constraining member (1308) to both fully cover the underlying balloons (1302, 1304) at one extreme of its travel and to allow full expansion of those balloons at the other end of its travel. In general, one way that this may be done is to form constraining member (1308) so that the portion of the constraining member constraining balloons (1302, 1304) has a length that is perhaps, up to at least twice the length of the balloons. The opening (1314) in the wall of catheter (1300) maybe situated at a distance approximately equal to the length of the balloons, from the end of the balloons. This is a highly conservative location for that opening (1314) and likely will result in the minimum of interference between the rapid exchange wire (1310) and the constraining member (1308).

This concept is shown in FIG. 62A where the length (1318) of the balloons is approximately equal to the distance (1320) between the end of the balloons and the opening (1314) in the catheter wall. This concept may also be seen from the top view depiction shown in FIG. 62B. FIG. 62B shows the length of the slot (1312) and the opening (1314) in the catheter wall. Note that the length of the slot (1312) also may approximate the length of the balloons. Also seen in FIG. 62B are some control members (1320) that allow placement of the constraining member (1308) properly over the balloons, when so needed. The constraining member (1308) may also be placed in a proper location by a long tubing member that may be considered a proximal extension of the constraining member (1308).

FIG. 62C shows the constraining member (1308) after it has been moved to the near extreme of its range of proximal movement. It will be noted that the slot (1312) still provides clearance to wire (1310) should such movement or should exchange be needed. Furthermore, it should be noted that the balloons, in particular, outer balloon (1302), is completely exposed and may be inflated along its entire length, if so needed.

This particular variation of the system is exceptionally flexible in allowing both cover of all or part of the balloon surface by the constraining member (1308) or complete removal of the constraining member from the balloon surface, if so desired. In either event, the rapid exchange wire remains available for removal or exchange.

Not shown in the drawings is a design feature that is sometimes valuable in catheters or core guide members having portions of the devices that differ significantly in stiffness, and, more pertinently, when a stiff area is adjacent an opening for an exchange system. In general, we sometimes use a “skived” section or a section that has a tapering diameter, often metallic, so that the region of the catheter between our more proximal section (that may be a hypotube) and the more distal sections has a better transition of stiffness and consequently do not kink. Again, because of the potential presence of an opening for the guidewire, the intersection region is flexible and this skived section allows creation of stiffness in that transitional region. This section also adds significant pushability to the catheter.

Multiballoon Catheter Having a Single Inflation Lumen

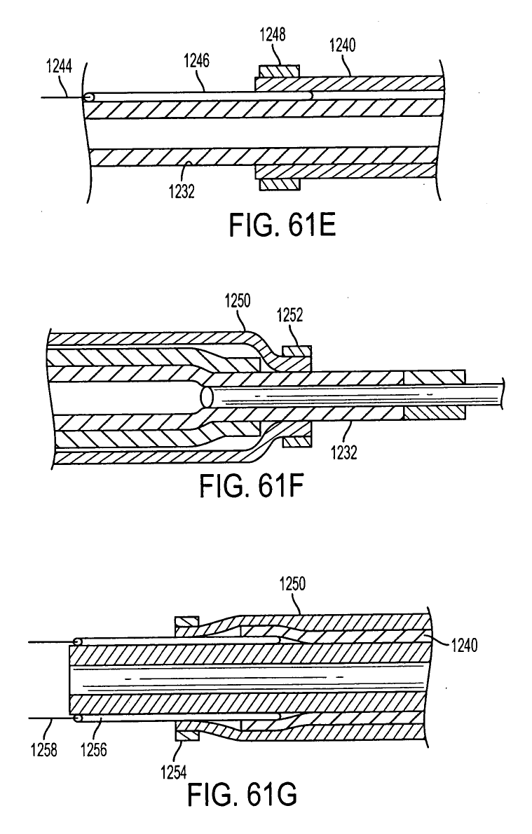

FIGS. 63A, 63B, 63C, and 63D show a balloon catheter employing a common, single, balloon inflation lumen in a multi balloon catheter.

FIG. 63A shows a balloon catheter system (1330) made up of a central core tubing member (1332) that may be used for the passage of guidewires and inflation fluid. The balloons shown here are the outer balloon (1334), the middle balloon (1336), and the inner balloon (1338). These balloons are radially adjacent each other and the ends of each of them adhere to the central catheter core member (1332). Each of the three balloons has a unique port or ports permitting fluid flow from the central lumen of catheter body (1332) into the balloons. Inner balloon (1338) has a passageway (1340); middle balloon (1336) includes a passageway (1342); and outer balloon (1334) has a more distal opening (1344). Each of the inflation fluid openings (1340, 1342, 1344) are open to inner lumen (1346) of catheter body (1332). Also shown in FIG. 63A is a core guide member (1348) that includes as one of its components an inflatable balloon (1350). Operation of the core guide member is, of course, shown elsewhere herein. Other small or narrow inflatable balloon catheters (or other sealing expandable members) able to extend down lumen (1346) of central core tubing member (1332) and seal that lumen to allow movement of inflation fluid through one or more of the openings to inflate the balloons are, of course, suitable as well in place of the depicted core guide member (1348).

FIG. 63B shows the system after core guide member (1348) has been moved distally down through catheter body (1332) so that the core guide member balloon (1350) is distal of the two sets of openings that extend into the inner balloon (1338) and the middle balloon (1336) and the core guide member balloon (1350) has been inflated to seal the catheter lumen (1346). That is to say, after the inflation of the core guide member balloon (1350), the opening (1340) leading into inner balloon (1338) and the opening (1342) into middle balloon (1336) are left uncovered. Pressurized inflation fluid passing through catheter lumen (1346) is then pressured into inner balloon (1338) and into middle balloon (1336). In this example, of course, outer balloon (1334) is not inflated. The pressure in the core guide member balloon (1350) typically exceeds the pressure in the catheter balloons.

FIG. 63C shows a condition similar to that shown in FIG. 63B, except that the core guide member (1348) has been moved a bit distally down catheter body (1332) so that the core guide member balloon (1350) blocks lumen (1346) distal of openings (1344) thereby allowing inflation of outer balloon (1334).

In summary, it should be apparent that each of the balloons shown in these drawings is sequentially inflatable by use of a mechanism such as the core guide member (1348) or, in this instance, other small inflatable balloon devices able to block the lumen (1346) of catheter body (1332).

FIG. 63D shows the balloon catheter system after the inner balloon (1338), the middle balloon (1336), and the outer balloon (1334) have been deflated. The deflation process would likely first include a step of lowering the pressure of the inflation fluid in the catheter lumen (1346). This step, by itself, allows balloon deflation. The core guide member balloon (1350) on core guide member (1348) is subsequently deflated. The fluid in each of the balloons escapes into the lumen (1346) of catheter (1332). The step of deflation may include suction of the inflation fluid out of the balloons.

Sterilized Kits

Various of the devices described here are best provided to the user in the form of kits. Such kits are common in the commerce surrounding disposable medical tools and devices. Two specific methods of sterilizing both the devices and kits include the use of non-ionizing radiation, typically after packaging, or a gas such as ethylene oxide (ETO) often in a multi step process involving sterilization of the device before packaging and sterilization of the packaging alone or with the device in place. In this way, a sterile package can be provided to the user-physician without the need for local sterilization with processes that may be harmful to medical devices containing thermoplastics. For instance, high temperature autoclaves using, e.g., steam or air at elevated temperatures, are not needed.

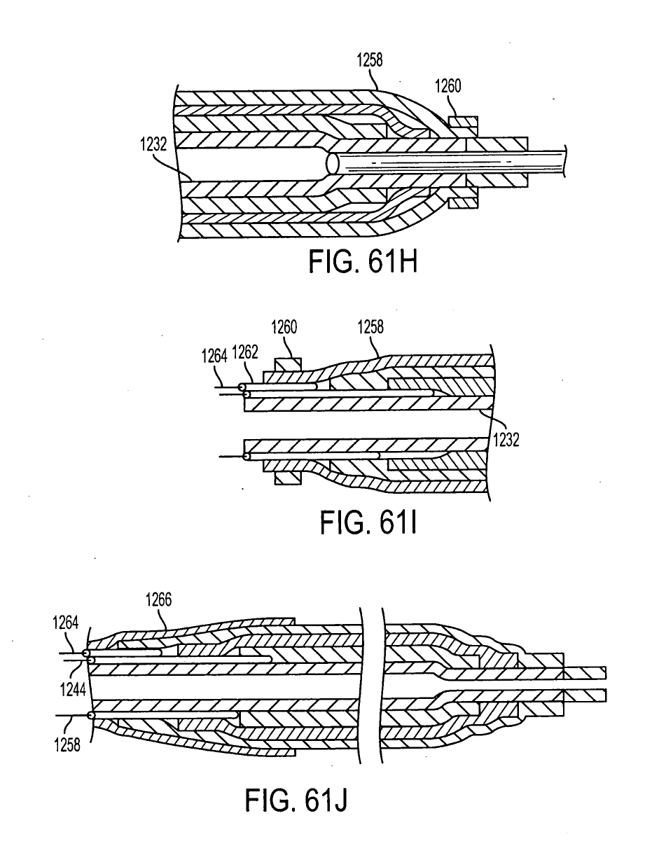

FIG. 64 shows one such kit (1360). In this variation, the packaging and the contents are sterilized (as are the remainder of the sterilized kits described below) and is in the form of a bubble pack (1362) sealed at the edge (1364) and having a bubble (1366) with a medical device, e.g., the described medical device system (1368), residing in that bubble (1366). Labeling (1370) is also shown.

In this kit (1360), the medical device typically would be included in a coil of tubing and would be removed from that coil prior to use. The system (1368) comprises a core guide member having an inflatable member and a constraining member slidable along the core guide member to adjust the length of the inflatable member. These devices are described above.

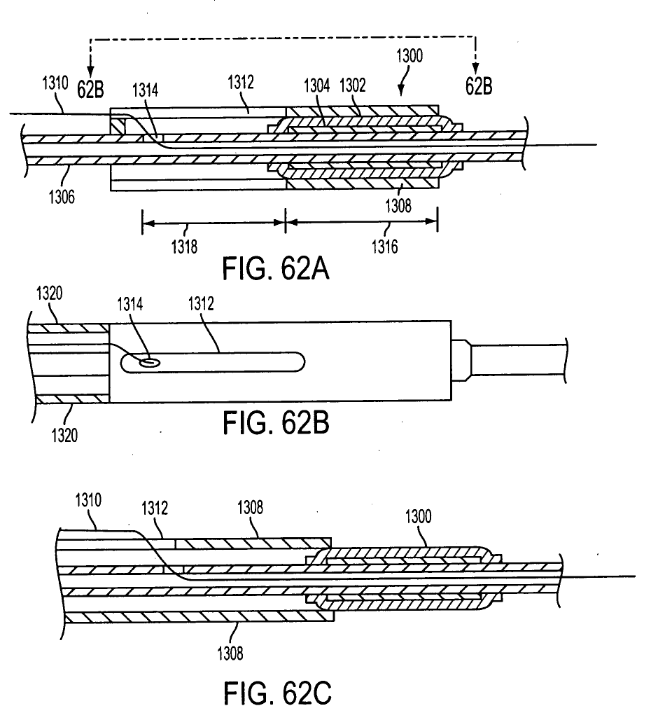

FIG. 65 shows a similar sterilized kit having device (1368) in a sterilized package (1372). Additionally, a second bubble containing an implant delivery sleeve (1374) is included in the kit. The implant sleeve may have one or more implants situated on or associated with the implant sleeve as desired.

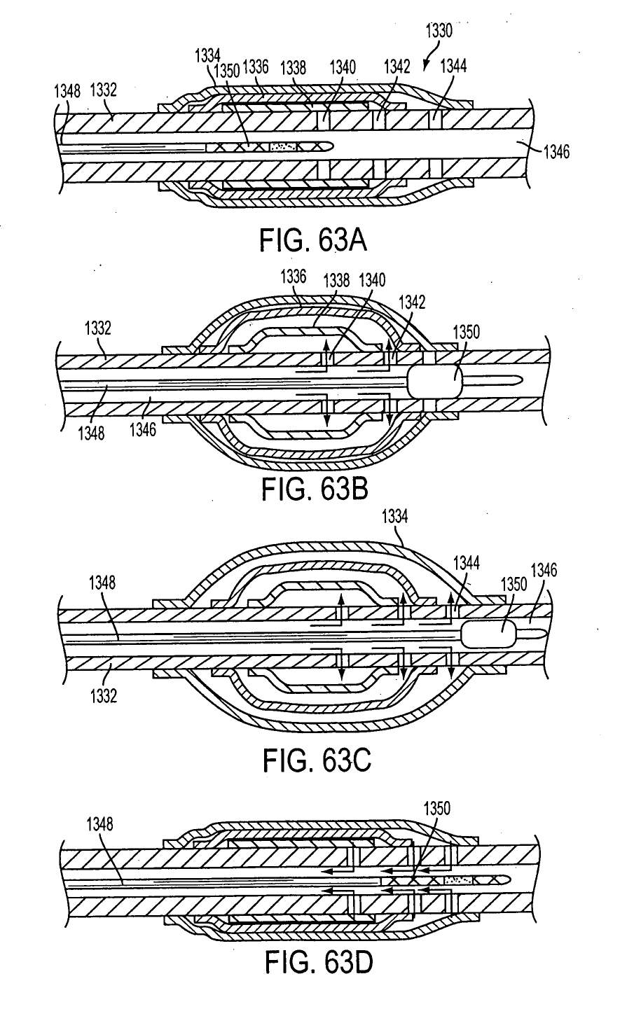

FIG. 66 shows another kit (1380) comprising an implant delivery sleeve (1382) and optionally a number of implants (1384) independently installable upon the implant sleeve. This sterilized kit (1380) provides the user with a variety of selectable implant, e.g., stent, sizes and diameters that may be associated with the sleeve for sequential or simultaneous placement using, perhaps, the sterilized kit and its included components (1368) shown in FIG. 64. Again, as noted, the kit and its contents are sterilized.

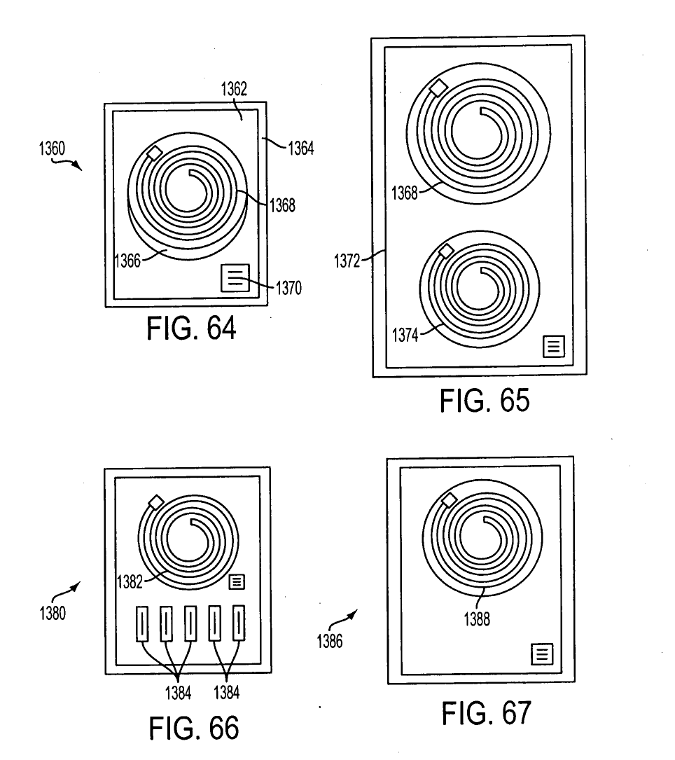

FIG. 67 shows a kit (1386) that has been sterilized and contains one of the tools (1388) described above, tools may compromise the stenosis cutting tool, the forming caul, or the others described above.

Another kit of particular use is made up of two balloon catheters, of the designs specified above, one of which, a first balloon catheter, includes one or more stents mounted on the balloon catheter for direct stenting. The other balloon catheter (or “second balloon catheter”) may be of a similar configuration to the first but may also comprise comparatively shorter balloon axial lengths and may be used, where desired, for such supplemental operations as pre-deployment angioplasty or post-deployment stent reformation (e.g., flaring or tapering of the stent). That is to say: the second balloon catheter may be used in conjunction with the first balloon catheter for a variety of steps before and after placement of the stent or stents carried upon that so-called first catheter. The second balloon catheter need not have a stent mounted on it, but as the need arises, may have such a stent. One procedure in which the second balloon catheter might be suitable would be in the tapering of a previously deployed stent to expand a portion of the stent, perhaps proximally in a coronary artery to meet the form of that artery's natural tapering, to a diameter larger than its initially deployed diameter. Additional kit components such as supplemental stents and stent deployment sleeves, a core guide member, guide catheter, and guidewire may also be suitable. Deflation aids and constraining members are applicable.

Any of the kits described here may also include one or more of the balloon catheters described here and in many cases, the core guide member will be replaced by a balloon catheter with a guidewire.

All publications, patents, and patent applications cited herein are hereby incorporated by reference in their entirety for all purposes to the same extent as if each individual publication, patent, or patent application were specifically and individually indicated to be so incorporated by reference. Although the foregoing devices have been described in some detail by way of illustration and example for purposes of clarity of understanding, it will be readily apparent to those of ordinary skill in the art in light of these teachings that certain changes and modifications may be made thereto without departing from the spirit and scope of the appended claims.

1. A system for treating a body comprising:
at least one controllably expandable assembly configured to be placed in a region in a human body requiring treating wherein said treating comprises at least one of: a step of expanding at least one body treating device to a selected diameter and a step of expanding at least one body treating device to a selected length, the controllably expandable assembly being configured to expand at least one of the at least one body treating devices, respectively, to more than one selected diameter or length, without removing the controllably expandable assembly from the human body.

2. The system of claim 1 further comprising at least one body treating device.

3. The system of claim 2 wherein at least one of the at least one body treating device comprises an implant.

4. The system of claim 2 wherein at least one of the at least one body treating device comprises a stent.

5. The system of claim 2 wherein at least one of the at least one body treating device comprises a stent-graft.

6. The system of claim 2 wherein at least one of the at least one body treating device comprises a sleeve.

7. The system of claim 6 wherein the sleeve contains a drug.

8. The system of claim 2 wherein at least one of the at least one body treating device comprises a movable sleeve.

9. The system of claim 2 wherein at least one of the at least one body treating device comprises a stent mounted upon a sleeve.

10. The system of claim 2 wherein at least one of the at least one body treating device comprises a stent mounted upon a movable sleeve.

11. The system of claim 2 wherein at least one of the at least one body treating device comprises a constraining sleeve.

12. The system of claim 1 wherein the controllably expandable assembly comprises a plurality of expandable members.

13. The system of claim 1 wherein the controllably expandable assembly comprises a plurality of inflatable members.

14. The system of claim 1 wherein the controllably expandable assembly comprises a plurality of inflatable balloons.

15. The system of claim 1 wherein the controllably expandable assembly comprises a plurality of inflatable balloons, at least two of which are radially adjacent each other.

16. The system of claim 15 wherein the controllably expandable assembly comprises a plurality of inflatable balloons, at least two of which are axially displaced from each other.

17. The system of claim 1 wherein the controllably expandable assembly comprises a plurality of inflatable balloons, at least two of which are axially displaced from each other.

18. The system of claim 1 wherein the controllably expandable assembly comprises a plurality of inflatable balloons, and further comprises an expansion-limiting sleeve, wherein the expansion-limiting sleeve is configured to limit the expansion of at least a portion of at least one of the plurality of inflatable balloons.

19. The system of claim 1 wherein the controllably expandable assembly comprises at least one artherotome.

20. The system of claim 19 wherein the controllably expandable assembly further comprises at least a sleeve.

21. A device for aiding in the contraction of an expandable member, comprising:
at least one expandable member having an inner surface expand and an outer surface, said expandable member further being contractible, and
at least one elastic contraction aid configured to expand upon at least partial expansion of at least one of the at least one expandable members, and to exert pressure upon at least one of the at least one expandable members while the contraction aid is at least partially expanded, and
wherein the at least one contraction aid is not expandable independently of the at least one expandable member.

22. The device of claim 21 wherein at least one of the at least one elastic contraction aid comprises a contraction ring.

23. The device of claim 22 wherein the contraction ring comprises a spring.

24. The device of claim 21 wherein at least one of the at least one elastic contraction aid comprises at least one elastic deflation aid and at least one of the at least one expandable members comprises an inflatable member.

25. The device of claim 24 wherein at least one of the at least one elastic deflation aids is adherent to the inner surface of at least one inflatable member.

26. The device of claim 24 wherein at least one of the at least one elastic deflation aids is adherent to the outer surface of at least one inflatable member.

27. The device of claim 24 wherein at least one of the at least one elastic deflation aids is adjacent to the outer surface of the inflatable member that causes the at least one of the at least one elastic deflation aids to expand.

28. The device of claim 24 wherein the at least one elastic deflation aid is configured to assist in deflating at least one inflatable member.

29. The device of claim 24 wherein the at least one elastic deflation aid is configured to assist in deflating more than one inflatable member.

30. The device of claim 24 wherein the at least one elastic deflation aid is configured to assist in returning at least one inflatable member towards its configuration prior to inflation.

31. The device of claim 24 wherein at least one of the at least one elastic deflation aids comprises deflation aid segments adherent to the outer surface of at least one inflatable member.

32. The device of claim 24 wherein at least one of the at least one elastic deflation aids comprises regions having differing relaxation rates.

33. The device of claim 32 wherein the at least one inflatable member comprises a noncompliant balloon having a pre-inflated folded form and the at least one elastic deflation aid is configured to return the balloon towards its pre-inflated folded form.

34. A device for setting deployed stent parameter values upon stent deployment, comprising:
at least one deployable stent, and
a user operable, deployed stent parameter value selector having more than one deployed stent parameter value selection, operable to expand one or more expandable members to expand at least one of the at least one deployable stents to a selected deployed stent parameter value.

35. The device of claim 34 wherein the device is manually operable by the user.

36. The device of claim 34 wherein the stent parameter value selector is manually operable by the user.

37. The device of claim 34 wherein the stent parameter values comprise deployed stent diameter.

38. The device of claim 34 wherein the stent parameter values comprise effective stent length.

39. The device of claim 34 wherein the one or more expandable members comprise more than one expandable members.

40. The device of claim 34 wherein the one or more expandable members comprise one or more inflatable balloons.

41. The device of claim 34 wherein the one or more expandable members comprise more than one radially adjacent inflatable balloons.

42. The device of claim 34 wherein the one or more expandable members comprise one or more axially displaced inflatable balloons.

43. The device of claim 41 wherein the one or more expandable members further comprise one or more axially displaced inflatable balloons.

44. The device of claim 34 wherein the one or more expandable members comprise one inflatable balloon.

45. The device of claim 34 wherein the stent parameter values comprise one or more values selected from deployed stent diameter and effective stent length.

46. The device of claim 34 wherein the effective stent length is greater than the length of the inflated expandable members.

47. The device of claim 34 wherein the one or more expandable members comprise more than one radially adjacent inflatable balloons, and the stent parameter value selector comprises a rotatable valve member, movable through a plurality of inflation positions, wherein at least one of the more than one inflatable balloons is inflatable by inflation fluid selected by selection of an inflation position.

48. The device of claim 34 wherein the one or more expandable members comprise a plurality of radially adjacent inflatable balloons and wherein at least one stent is in contact with at least one of the plurality of radially adjacent inflatable balloons.

49. The device of claim 34 wherein the one or more expandable members comprise one or more axially displaced inflatable balloons and wherein at least one stent is in contact with at least one of the plurality of axially displaced inflatable balloons.

50. The device of claim 43 wherein at least one stent is in contact with at least one of the plurality of axially displaced inflatable balloons.

51. The device of claim 48 wherein the one or more expandable members further comprise a comparatively lower profile distal inflatable balloon.

52. The device of claim 43 further comprising an outer elastic member radially adjacent to at least one of the more than one of inflatable balloons, configured to restore the at least one inflatable balloon to a lower profile upon deflation of the inflatable balloons.

53. The device of claim 43 further comprising an outer elastic member radially adjacent to at least one of the more than one of inflatable balloons, configured to push inflation fluid out of said inflatable balloons upon deflation of the inflatable balloons.

54. The device of claim 34 wherein the deployed stent parameter value selector is further cooperative with the expandable members so that when a change of selection is made, the expansion state of previously expanded expandable members is maintained during and after the selection.

55. The device of claim 54 further comprising one or more check valves for maintaining the expansion state of the previously expanded expandable members during and after the change in selection.

56. The device of claim 34 further comprising a single lumen for individually expanding the one or more expandable members.

57. The device of claim 34 further comprising multiple lumens for individually expanding the one or more expandable members.

58. The device of claim 34 comprising more than one expandable members, more than one lumen in fluid communication with the selector for individually expanding the one or more expandable members, and wherein at least one of the expandable members is a non-compliant balloon.

59. The device of claim 58 wherein all of the at least one of the expandable members are non-compliant balloons.

60. A device for setting artherotome parameter values upon artherotome extension, comprising:
at least one extendible artherotome, and
a user operable, artherotome parameter value selector having more than one artherotome parameter value selection, operable to expand one or more expandable members to expand the artherotome to a selected artherotome parameter value.

61. The device of claim 60 wherein the device is manually operable by the user.

62. The device of claim 60 wherein the artherotome parameter value selector is manually operable by the user.

63. The device of claim 60 wherein the artherotome parameter values comprise artherotome diameter.

64. The device of claim 60 wherein the artherotome parameter values comprise effective artherotome length.

65. The device of claim 60 wherein the one or more expandable members comprise more than one expandable members.

66. The device of claim 60 wherein the one or more expandable members comprise one or more inflatable balloons.

67. The device of claim 60 wherein the one or more expandable members comprise more than one radially adjacent inflatable balloons.

68. The device of claim 60 wherein the one or more expandable members comprise one or more axially displaced inflatable balloons.

69. The device of claim 67 wherein the one or more expandable members further comprise one or more axially displaced inflatable balloons.

70. The device of claim 60 wherein the one or more expandable members comprise one inflatable balloon.

71. The device of claim 60 wherein the artherotome parameter values comprise one or more values selected from artherotome diameter and effective artherotome length.

72. The device of claim 60 wherein the effective artherotome length is greater than the length of the inflated expandable members.

73. The device of claim 60 wherein the one or more expandable members comprise more than one radially adjacent inflatable balloons, and the artherotome parameter value selector comprises a rotatable valve member, movable through a plurality of inflation positions, wherein at least one of the more than one inflatable balloons is inflatable by inflation fluid selected by selection of an inflation position.

74. The device of claim 60 wherein the one or more expandable members comprise a plurality of radially adjacent inflatable balloons and wherein at least one artherotome is in contact with at least one of the plurality of radially adjacent inflatable balloons.

75. The device of claim 60 wherein the one or more expandable members comprise one or more axially displaced inflatable balloons and wherein at least one artherotome is in contact with at least one of the plurality of axially displaced inflatable balloons.

76. The device of claim 69 wherein at least one artherotome is in contact with at least one of the plurality of axially displaced inflatable balloons.

77. The device of claim 69 wherein the one or more expandable members further comprise a comparatively lower profile distal inflatable balloon.

78. The device of claim 66 further comprising an outer elastic member radially adjacent to at least one of the more than one of inflatable balloons, configured to restore the at least one inflatable balloon to a lower profile upon deflation of the inflatable balloons.

79. The device of claim 66 further comprising an outer elastic member radially adjacent to at least one of the more than one of inflatable balloons, configured to push inflation fluid out of said inflatable balloons upon deflation of the inflatable balloons.

80. The device of claim 60 wherein the artherotome parameter value selector is further cooperative with the expandable members so that when a change of selection is made, the expansion state of previously expanded expandable members is maintained during and after the selection.

81. The device of claim 80 further comprising one or more check valves for maintaining the expansion state of the previously expanded expandable members during and after the change in selection.

82. The device of claim 60 further comprising a single lumen for individually expanding the one or more expandable members.

83. The device of claim 60 further comprising multiple lumens for individually expanding the one or more expandable members.

84. The device of claim 60 comprising more than one expandable member, more than one lumen in fluid communication with the selector for individually expanding the one or more expandable members, and wherein at least one of the expandable members is a non-compliant balloon.

85. The device of claim 66 wherein all of the at least one of the expandable members are non-compliant balloons.

86. A stent delivery system comprising:
at least one controllably expandable assembly configured to be placed in a region in a human body requiring stenting and to deliver at least one stent, the controllably expandable assembly being configured to expand at least one of the at least one stents to more than one selected length without removing the expandable assembly from the human body, and
the at least one stent.

87. The stent delivery system of claim 86 wherein one or more of the at least one stent comprises a stent-graft.

88. The stent delivery system of claim 86 wherein the controllably expandable assembly comprises a plurality of inflatable members.

89. The stent delivery system of claim 86 wherein the controllably expandable assembly comprises a plurality of inflatable balloons.

90. The stent delivery system of claim 86 wherein the controllably expandable assembly comprises a plurality of inflatable balloons, at least two of which are radially adjacent each other.

91. The stent delivery system of claim 86 wherein the controllably expandable assembly comprises a plurality of inflatable balloons, at least two of which are axially displaced from each other.

92. The stent delivery system of claim 90 wherein the controllably expandable assembly comprises a plurality of inflatable balloons, at least two of which are axially displaced from each other.

93. The stent delivery system of claim 89 wherein the at least one stent is in contact with at least one of the plurality of inflatable balloons.

94. The stent delivery system of claim 89 wherein the at least one stent is mounted upon a sleeve.

95. The stent delivery system of claim 89 wherein the at least one stent is mounted upon a movable sleeve.

96. The stent delivery system of claim 89 where each of the plurality of inflatable balloons independently comprises a compliant balloon, semi-compliant balloon, or non-compliant balloon.

97. The stent delivery system of claim 86 wherein the controllably expandable assembly comprises a plurality of inflatable balloons, and further comprises an expansion-limiting sleeve, wherein the expansion-limiting sleeve is configured to limit the expansion of at least a portion of at least one of the plurality of inflatable balloons.

98. The stent delivery system of claim 97 wherein the expansion-limiting sleeve is not movable with respect to the controllably expandable assembly after insertion into the human body by a user.

99. The stent delivery system of claim 86 wherein the controllably expandable assembly further comprises an expansion-limiting sleeve, wherein the expansion-limiting sleeve is configured to limit the expansion of at least a portion of at the least one stent.

100. The stent delivery system of claim 86 wherein the at least one stent is more than one stent.

101. The stent delivery system of claim 89 wherein the one or more expandable members further comprise a comparatively lower profile distal inflatable balloon.

102. The stent delivery system of claim 89 further comprising an outer elastic member radially adjacent to at least one of the more than one of inflatable balloons, configured to restore the at least one inflatable balloon to a lower profile upon deflation of the inflatable balloons.

103. The stent delivery system of claim 89 further comprising an outer elastic member radially adjacent to at least one of the more than one of inflatable balloons, configured to push inflation fluid out of said inflatable balloons upon deflation of the inflatable balloons.

104. The stent delivery system of claim 86 further comprising a single lumen for individually expanding the one or more expandable members.

105. The stent delivery system of claim 86 further comprising multiple lumens for individually expanding the one or more expandable members.

106. A stent delivery system comprising:
at least one controllably expandable assembly configured to be placed in a region in a human body requiring stenting and to deliver at least one stent, the controllably expandable assembly being configured to expand at least one of the at least one stent to more than one selected diameter without removing the expandable assembly from the human body, and
the at least one stent.

107. The stent delivery system of claim 106 wherein one or more of the at least one stents comprises a stent-graft.

108. The stent delivery system of claim 106 wherein the controllably expandable assembly comprises a plurality of inflatable members.

109. The stent delivery system of claim 108 wherein the controllably expandable assembly comprises a plurality of inflatable balloons.

110. The stent delivery system of claim 106 wherein the controllably expandable assembly comprises a plurality of inflatable balloons, at least two of which are radially adjacent each other.

111. The stent delivery system of claim 106 wherein the controllably expandable assembly comprises a plurality of inflatable balloons, at least two of which are axially displaced from each other.

112. The stent delivery system of claim 110 wherein the controllably expandable assembly comprises a plurality of inflatable balloons, at least two of which are axially displaced from each other.

113. The stent delivery system of claim 109 wherein the at least stent is in contact with at least one of the plurality of inflatable balloons.

114. The stent delivery system of claim 110 wherein the at least one stent is mounted upon a sleeve.

115. The stent delivery system of claim 109 wherein the at least one stent is mounted upon a movable sleeve.

116. The stent delivery system of claim 113 where each of the plurality of inflatable balloons independently comprises a compliant balloon, semi-compliant balloon, or non-compliant balloon.

117. The stent delivery system of claim 109 wherein the controllably expandable assembly further comprises an expansion-limiting sleeve, wherein the expansion-limiting sleeve is configured to limit the expansion of at least a portion of at least one of the plurality of inflatable balloons.

118. The stent delivery system of claim 1 17 wherein the expansion-limiting sleeve is not movable with respect to the controllably expandable assembly after insertion into the human body by a user.

119. The stent delivery system of claim 106 wherein the controllably expandable assembly further comprises an expansion-limiting sleeve, wherein the expansion-limiting sleeve is configured to limit the expansion of at least a portion of at the least one stent.

120. The stent delivery system of claim 106 wherein the at least one stent is more than one stent.

121. The stent delivery system of claim 110 wherein the one or more expandable members further comprise a comparatively lower profile distal inflatable balloon.

122. The stent delivery system of claim 110 further comprising an outer elastic member radially adjacent to at least one of the more than one of inflatable balloons, configured to restore the at least one inflatable balloon to a lower profile upon deflation of the inflatable balloons.

123. The stent delivery system of claim 110 further comprising an outer elastic member radially adjacent to at least one of the more than one of inflatable balloons, configured to push inflation fluid out of said inflatable balloons upon deflation of the inflatable balloons.

124. The stent delivery system of claim 106 further comprising a single lumen for individually expanding the one or more expandable members.

125. The stent delivery system of claim 106 further comprising multiple lumens for individually expanding the one or more expandable members.

126. A controllable balloon expansion system comprising:
a.) a plurality of expandable balloons, and
b.) a valving device having more than one valve opening, movable through a plurality of inflation positions, wherein at least one of the plurality of expandable balloons is expandable by inflation fluid flowing through at least one valve opening associated with a valve position.

127. The balloon expansion system of claim 126 wherein the plurality of expandable balloons comprise inflatable balloons.

128. The balloon expansion system of claim 126 wherein the plurality of expandable balloons comprise a balloon catheter.

129. The balloon expansion system of claim 126 wherein the plurality of expandable balloons comprise a core guide member.

130. The balloon expansion system of claim 126 wherein the valving device is manually movable by a user.

131. The balloon expansion system of claim 126 wherein when a new inflation position is chosen, the inflation of a previously inflated balloon is substantially maintained.

132. The balloon expansion system of claim 126 further comprising at least one check valve configured to substantially maintain the inflation of a previously inflated balloon wherein a new inflation position is chosen.

133. The balloon expansion system of claim 126 wherein the valving device is configured to inflate at least one balloon at an inflation position, and wherein a set of adjacent positions is configured to inflate a set of balloons to different diameters.

134. The balloon expansion system of claim 133 further comprising at least one stent configured to be deployed by the set of balloons of different diameters.

135. The balloon expansion system of claim 133 wherein the valving device is configured to inflate at least one balloon at an inflation position, and wherein a set of adjacent positions is configured to inflate a set of balloons to increasing diameters.

136. The balloon expansion system of claim 135 further comprising at least one stent configured to be deployed by the set of balloons of increasing diameters.

137. The balloon expansion system of claim 133 further comprising at least one atherotome configured to be deployed by the set of balloons of different diameters.

138. The balloon expansion system of claim 126 wherein the valving device is configured to inflate at least one balloon at an inflation position, and wherein a set of adjacent positions is configured to inflate a set of balloons resulting in a different effective length of inflated balloons.

139. The balloon expansion system of claim 138 further comprising at least one stent configured to be deployed by the set of balloons resulting in a different effective length of inflated balloons.

140. The balloon expansion system of claim 139 further comprising more than one stent configured to be deployed by the set of balloons resulting in an increasing length of inflated balloons.

141. The balloon expansion system of claim 126 wherein the valving device further comprises a deflation position configured to allow inflation fluid to flow out of each of the balloons.

142. The balloon expansion system of claim 133 wherein the valving device is manually operable by a user through the set of adjacent positions to inflate a set of balloons to a selected diameter.

143. The balloon expansion system of claim 136 wherein the valving device is manually operable by a user through the set of adjacent positions to inflate a set of balloons and to deploy at least one stent to a selected diameter.

144. The balloon expansion system of claim 133 wherein the valving device is manually operable by a user through the set of adjacent positions to inflate a set of balloons resulting in a different length of inflated balloons.

145. The balloon expansion system of claim 144 wherein the valving device is manually operable by a user through the set of adjacent positions to inflate a set of balloons resulting in an increasing length of inflated balloons.

146. The balloon expansion system of claim 144 wherein the valving device is manually operable by a user through the set of adjacent positions to inflate a set of balloons and to deploy at least one stent to a selected length.

147. The balloon expansion system of claim 133 wherein the valving device is further configured to inflate at least one balloon at an inflation position, and wherein a set of adjacent positions is configured to inflate a set of balloons resulting in a different effective length of inflated balloons.

148. The balloon expansion system of claim 147 further comprising at least one stent configured to be deployed by the set of balloons resulting in a different effective length of inflated balloons.

149. The balloon expansion system of claim 126 wherein the valving device plurality of inflation positions, is configured to expand a plurality of expandable balloons resulting in at least one of a.) different effective lengths and b.) selected diameter of inflated balloons.

150. The balloon expansion system of claim 126 wherein the valving device is further cooperative with the expandable balloons so that when a change of selection is made, the expansion state of previously expanded expandable balloons is maintained during and after the selection.

151. The balloon expansion system of claim 150 further comprising one or more check valves for maintaining the expansion state of the previously expanded expandable balloons during and after the change in selection.

152. The balloon expansion system of claim 126 comprising more than one expandable members, more than one lumen in fluid communication with the selector for individually expanding the one or more expandable members, and wherein at least one of the expandable members is a non-compliant balloon.

153. The balloon expansion system of claim 152 wherein all of the at least one of the expandable members are non-compliant balloons.

154. The balloon expansion system of claim 126 further comprising a single lumen for individually expanding each of the plurality of expandable balloons.

155. The balloon expansion system of claim 126 further comprising multiple lumens, at least one is configured to individually expand more than one of the plurality of expandable balloons.

156. The balloon expansion system of claim 126 further comprising multiple lumens for individually expanding each of the plurality of expandable balloons.

157. A medical device system having an adjustable-length inflatable member comprising:
a core guide member having at least one inflation area, a proximal end, a distal end, a core passageway that is fluidly connected with the at least one inflation area, and is closable distally of the at least one inflation area; having at least one inflatable member with a length, the inflatable member surrounding at least a portion of one of the at least one inflation area, and that is sealingly connected to the core guide member to form an inflatable region in fluid connection with the core passageway; and
at least one constraining member longitudinally slidable along the core guide member, having a distal end, and wherein the constraining member is configured to slide upon the inflatable member, to constrain inflation of the inflatable member proximally of the constraining member distal end, and to permit inflation of the inflatable member distally of the constraining member distal end, whereby the longitudinal movement of the constraining member adjusts the length of the inflatable member available for inflation.

158. The medical device system of claim 157 where the inflatable member is sealingly connected to the core guide member proximally and distally of the at least one inflation area.

159. The medical device system of claim 157 where the core passageway extends from the core guide member proximal end to the at least one inflation area.

160. The medical device system of claim 157 where the core passageway is closed distally of the at least one inflation area.

161. The medical device system of claim 157 where the core passageway is at least partially open distally of the at least one inflation area.

162. The medical device system of claim 157 where the core passageway has an opening outside of the at least one inflation area with a size selected to allow a controlled leakdown.

163. The medical device system of claim 157 where the core guide member passageway is in fluid connection with the core guide member exterior outside of the inflation area.

164. The medical device system of claim 157 where the at least one inflatable member comprises elastomeric material.

165. The medical device system of claim 157 where the at least one inflatable member comprises non-elastomeric material.

166. The medical device system of claim 157 where the at least one inflatable member comprises a material selected to permit permeation of inflation fluid through the inflatable member.

167. The medical device system of claim 157 where the at least one inflatable member comprises a plurality of inflatable members mounted radially adjacent each other.

168. The medical device system of claim 157 where the at least one inflatable member comprises a plurality of inflatable members axially displaced from each other.

169. The medical device system of claim 167 where the at least one inflatable member further comprises a comparatively lower profile distal inflatable member section.

170. The medical device system of claim 169 where the distal inflatable member section has a diameter no more than about 0.014 inches.

171. The medical device system of claim 169 where the distal inflatable member section has a diameter no more than about 0.018 inches.

172. The medical device system of claim 169 where the plurality of inflatable members has a diameter no more than about 0.035 inches.

173. The medical device system of claim 169 where the distal inflatable member section is a compliant balloon.

174. The medical device system of claim 169 where the distal inflatable member section is a semi-compliant balloon.

175. The medical device system of claim 169 where the distal inflatable member section is a non-compliant balloon.

176. The medical device system of claim 169 further comprising an outer layer comprising an elastic sleeve radially adjacent at least a portion of the distal inflatable member section or the section comprising a plurality of inflatable members, to restore the at least one section to a lower profile.

177. The medical device system of claim 169 further comprising an outer elastic member radially adjacent at least a portion of the distal inflatable member section or the section comprising a plurality of inflatable members, to push inflation fluid from the distal inflatable member section or the section comprising a plurality of inflatable members.

178. The medical device system of claim 169 further comprising an outer elastic member radially adjacent at least a portion of the distal inflatable member section or the section comprising a plurality of inflatable members, to restore the at least one section to a lower profile.

179. The medical device system of claim 157 where the core guide member further comprises a distally located guide tip.

180. The medical device system of claim 157 wherein the core guide member comprises a metallic material.

181. The medical device system of claim 157 wherein the core guide member comprises a polymeric material.

182. The medical device system of claim 157 wherein the core guide member has a low profile.

183. The medical device system of claim 182 wherein the diameter of the core guide member is less than about 0.100 inches.

184. The medical device system of claim 183 wherein the diameter of the core guide member is less than about 0.030 inches.

185. The medical device system of claim 183 wherein the diameter of the core guide member is less than about 0.014 inches.

186. The medical device system of claim 157 wherein the distal end of the core guide member is closed.

187. The medical device system of claim 157 wherein at least one core guide member passageway is fluidly connected to the inflation area through at least one opening comprising at least one slit in the core guide member.

188. The medical device system of claim 157 wherein at least one core guide member passageway is fluidly connected to the inflation area through at least one opening comprising more than one slit in the core guide member.

189. The medical device system of claim 188 wherein the at least one slit is helical.

190. The medical device system of claim 157 wherein at least one core guide member passageway is fluidly connected to the inflation area through at least one opening comprising at least one hole in the core guide member.

191. The medical device system of claim 157 further comprising a catheter.

192. The medical device system of claim 157 further comprising a balloon catheter.

193. The medical device system of claim 157 further comprising a balloon catheter having more than one inflatable member.

194. The medical device system of claim 157 further comprising a balloon catheter having at least two inflatable members mounted radially adjacent each other.

195. The medical device system of claim 157 further comprising a balloon catheter having more than two inflatable members mounted radially adjacent each other.

196. The medical device system of claim 157 further comprising a balloon catheter having at least two inflatable members mounted longitudinally adjacent each other.

197. The medical device system of claim 196 further comprising a balloon catheter having at least two inflatable members mounted radially adjacent each other.

198. The medical device system of claim 196 further comprising a balloon catheter having at least two inflatable members mounted radially adjacent each other and further comprising one or more atherotomes mounted to extend radially upon inflation of at least one of the at least two inflatable members.

199. The medical device system of claim 157 further comprising at least one stenting structure.

200. The medical device system of claim 199 wherein the at least one stenting structure is in contact with the inflatable member.

201. The medical device system of claim 157 further comprising a plurality of stenting structures.

202. The medical device system of claim 157 further comprising a plurality of stenting structures mounted upon a stent delivery sleeve and wherein the stent delivery sleeve comprises at least one sleeve having an interior longitudinal opening and wherein the sleeve is configured to deploy those stenting devices independently without substantially affecting adjacent stenting devices.

203. The medical device system of claim 202 wherein the stent delivery sleeve is slidable upon the at least one constraining member.

204. The medical device system of claim 202 wherein the stent delivery sleeve is slidable beneath the at least one constraining member.

205. The medical device system of claim 202 wherein the sleeve is configured to deploy at least one of those stenting devices independently by inflating the inflatable member in the interior longitudinal opening.

206. The medical device system of claim 202 wherein the sleeve is configured to allow self-deployment of at least one of those stenting devices independently.

207. The medical device system of claim 202 wherein the stent delivery sleeve comprises at least one filamentary sleeve.

208. The medical device system of claim 202 wherein the delivery sleeve comprises at least one sleeve comprising an elastic membrane.

209. A medical device system for delivering at least one stent comprising:
a core guide member having a core guide member body, at least one expansion assembly, a proximal end, a distal end, a core passageway that is fluidly connected with the at least one expansion assembly and is closable distally of the at least one expansion assembly; the expansion assembly at least partially surrounding the core guide member body and comprising at least one inflatable member with a length and diameter, the inflatable member being sealingly connected to the core guide member body to form an inflatable region in fluid connection with the core passageway; and
at least one stent in contact with the expansion assembly.

210. The medical device system of claim 209 where the expansion assembly comprises a plurality of inflatable members.

211. The medical device system of claim 209 where the expansion assembly comprises a plurality of balloons.

212. The medical device system of claim 209 where the at least one inflatable member comprises a plurality of inflatable members mounted radially adjacent each other.

213. The medical device system of claim 209 where the at least one inflatable member further comprises a comparatively lower profile distal inflatable member section.

214. The medical device system of claim 210 further comprising a deflation aid configured to assist in deflation of the at least one inflatable member.

215. The medical device system of claim 212 where the deflation aid comprises an outer elastic member radially adjacent to at least one of the plurality of inflatable members, configured to restore the at least one inflatable member to a lower profile upon deflation of the at least one inflatable member.

216. The medical device system of claim 212 where the deflation aid comprises an outer elastic member radially adjacent to at least one of the plurality of inflatable members, configured to push inflation fluid out of the at least one inflatable member upon deflation of the at least one inflatable member.

217. The medical device system of claim 212 where the deflation aid is configured to prevent collapse of at least one inflation lumen during deflation of the at least one inflatable member.

218. The medical device system of claim 213 where the distal inflatable member section has a diameter no more than about 0.014 inches.

219. The medical device system of claim 213 where the distal inflatable member section has a diameter no more than about 0.018 inches.

220. The medical device system of claim 210 where the plurality of inflatable members has a diameter no more than about 0.035 inches.

221. The medical device system of claim 213 where the distal inflatable member section comprises a compliant balloon, semi-compliant balloon, or non-compliant balloon.

222. The medical device system of claim 209 where the core guide member further comprises a distally located guide tip.

223. The medical device system of claim 209 wherein the core guide member body comprises a metallic material or a polymeric material.

224. The medical device system of claim 209 wherein the distal end of the core guide member is closed.

225. The medical device system of claim 209 further comprising a plurality of stents.

226. The medical device system of claim 225 wherein the plurality of stents is in contact with at least one of the inflatable members.

227. The medical device system of claim 225 further comprising a sleeve mounted to cover at least one of the plurality of stents in contact with at least one of the inflatable members and wherein the sleeve is configured to remain substantially fixed in position with respect to the core guide member after placement of the core guide member in the body and to prevent expansion of the covered at least one stent.

228. The medical device system of claim 209 further comprising at least one constraining member longitudinally slidable along the core guide member, having a distal end, and wherein the constraining member is configured to be remotely slidable with respect to the core guide member by a user after the core guide member and constraining member are introduced in the body, to slide upon the inflatable member, to constrain inflation of the inflatable member proximally of the constraining member distal end, and to permit inflation of the inflatable member distally of the constraining member distal end.

229. The medical device system of claim 228 wherein the longitudinal movement of the constraining member adjusts the length of the inflatable member available for inflation.

230. The medical device system of claim 229 wherein the constraining member is adapted to slide beneath the stenting structure.

231. The medical device system of claim 209 where each inflatable members is independently inflatable.

232. A combination stent delivery sleeve and balloon catheter medical device system comprising:
a balloon catheter having more than one inflatable member,
at least one filamentary sleeve having an interior longitudinal opening and wherein the filaments are of a size, flexibility, and shape and comprising materials appropriate a.) to support stenting devices and b.) to deploy those stenting devices independently without substantially affecting adjacent stents, and
at least one stenting device mounted exterior to the at least one filamentary sleeve.

233. The combination medical device system of claim 232 wherein the balloon catheter comprises at least two inflatable members mounted radially adjacent each other.

234. The combination medical device system of claim 232 wherein the balloon catheter comprises more than two inflatable members mounted radially adjacent each other.

235. The combination medical device system of claim 232 wherein the balloon catheter comprises at least two inflatable members mounted longitudinally adjacent each other.

236. The combination medical device system of claim 235 wherein the balloon catheter further comprises at least two inflatable members mounted radially adjacent each other.

237. The combination medical device system of claim 232 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

238. The combination medical device system of claim 232 further comprising a guide wire.

239. The combination medical device system of claim 232 where the filamentary sleeve further is configured to deploy those stenting devices independently by inflating at least one of the balloon catheter inflatable members in the interior longitudinal opening and to return to a pre-deployment shape without substantial plastic deformation.

240. The combination medical device system of claim 232 where the filamentary sleeve has a substantially constant diameter.

241. The combination medical device system of claim 232 where the filamentary sleeve does not have a substantially constant diameter.

242. The combination medical device system of claim 232 where the at least one stenting device comprises more than one stenting device of which at least one is deployable using a inflatable member.

243. The combination medical device system of claim 232 where the at least one stenting device of which at least one is a self-expanding stenting device.

244. The combination medical device system of claim 243 further comprising a removable retainer configured to controllably allow the at least one self-expanding stenting device to individually self-deploy.

245. The combination medical device system of claim 232 where the filaments comprise a super-elastic alloy.

246. The combination medical device system of claim 232 where the filaments comprise nitinol.

247. The combination medical device system of claim 232 where the filaments comprise a stainless steel.

248. The combination medical device system of claim 232 where the filaments comprise wire.

249. The combination medical device system of claim 232 where the filaments comprise ribbon.

250. The combination medical device system of claim 232 where the at least one stenting device comprises more than one stenting device each having substantially the same length.

251. The combination medical device system of claim 232 where the at least one stenting device comprises more than one stenting device and not having substantially the same length.

252. The combination medical device system of claim 232 where the at least one stenting device comprises more than one stenting device and not having substantially the same expanded diameter.

253. The combination medical device system of claim 232 where the filamentary sleeve comprises a braid.

254. The combination medical device system of claim 232 where the filamentary sleeve comprises a woven or knitted braid.

255. The combination medical device system of claim 232 further comprising an elongate position control member attached to an end of one of the at least one filamentary sleeve and configured to allow a user to position the sleeve at a selected site.

256. The combination medical device system of claim 232 comprising at least one filamentary sleeve having at least one stenting device joinable to another filamentary sleeve having at least one stenting device.

257. The combination medical device system of claim 232 comprising more than one filamentary sleeve each having at least one stenting device joined to another filamentary sleeve having at least one stenting device.

258. The combination medical device system of claim 257 comprising more than one filamentary sleeve having at least one stenting device joined to another filamentary sleeve having at least one stenting device and further joined to an elongate position control member attached to an end of one of the filamentary sleeves.

259. The combination medical device system of claim 232 where the at least one stenting device further comprises at least one biologically active agent.

260. The combination medical device system of claim 259 where the at least one biologically active agent comprises one or more immunosuppressants.

261. The combination medical device system of claim 260 where the one or more immunosuppressants comprise sirolimus, everolimus, tacrolimus, or their mixtures.

262. The combination medical device system of claim 260 where the one or more immunosuppressants comprise one of cyclosporins, azathioprines, and corticosteroids.

263. The combination medical device system of claim 232 where the at least one stenting device further comprises a releasable biologically active agent selected from the group consisting of anti-proliferation agents, anti-inflammatory agents, antibiotics, and immunosuppressants.

264. The combination medical device system of claim 232 where the at least one stenting device further comprises a releasable biologically active agent selected from the group consisting of paclitaxel, methotrexate, batimastal, doxycycline, tetracycline, rapamycin, actinomycin, dexamethosone, methyl prednisolone, prednisolone, nitroprussides, estrogen, estradiols, and their mixtures.

265. The combination medical device system of claim 232 further comprising a sleeve containing a at least one biologically active agent.

266. The combination medical device system of claim 265 where the at least one biologically active agent comprises one or more immunosuppressants.

267. The combination medical device system of claim 232 further comprising a guide wire.

268. The combination medical device system of claim 232 wherein the at least one stenting device is mounted exterior to the at least one filamentary sleeve, wherein at least two of the at least one stenting devices are joinable to each other.

269. The combination medical device system of claim 232 adapted for use with a rapid exchange mechanism.

270. An artherotomy system comprising:
at least one controllably expandable assembly configured to be placed in a region in a human body requiring artherotomy and to provide at least one artherotome for such artherotomy, the controllably expandable assembly comprising at least one artherotome, and being configured to expand to more than one selected diameter without removing the expandable assembly from the human body and to effect such artherotomy.

271. The artherotomy system of claim 270 wherein the controllably expandable assembly comprises a plurality of inflatable balloons.

272. The artherotomy system of claim 270 wherein the controllably expandable assembly comprises a plurality of inflatable balloons, at least two of which are radially adjacent each other.

273. The artherotomy system of claim 270 wherein the controllably expandable assembly comprises a plurality of inflatable balloons, at least two of which are axially displaced from each other.

274. The artherotomy system of claim 270 wherein the at least one controllably expandable assembly comprises a member selected from the group consisting of balloon catheters and core guide members.

275. The artherotomy system of claim 271 wherein the controllably expandable assembly further comprises an expansion-limiting sleeve, wherein the expansion-limiting sleeve is configured to limit the expansion of at least a portion of at least one of the plurality of inflatable balloons.

276. The artherotomy system of claim 275 wherein the expansion-limiting sleeve is not movable with respect to the artherotomy system after insertion into the human body by a user.

277. The artherotomy system of claim 270 wherein the controllably expandable assembly further comprises an expansion-limiting sleeve, wherein the expansion-limiting sleeve is configured to limit the expansion of at least a portion of at least one artherotome.

278. The artherotomy system of claim 277 wherein the expansion-limiting sleeve is configured for use with a rapid exchange system.

279. The artherotomy system of claim 270 configured for use with a rapid exchange system.

280. A stenotic incision tool for cutting stenoses found in vascular lumen, comprising:
an atherotome holding member having a longitudinal axis, comprising:
a.) an inner substrate having a passageway, a radius, and an outer surface, the substrate being adapted to cooperate with at least a movable inflatable member and to expand to extend at least one of a plurality of atherotomes in a substantially radial direction when a movable inflatable member is inflated in the passageway,
b.) an outer member having an outer surface, and
c.) a plurality of atherotomes having longitudinal axes, fixedly and movably mounted to said inner substrate, and each of the plurality of atherotomes adapted to extend from the outer surface substantially parallel to the holding member longitudinal axis when the movable at least one inflatable member is inflated in the passageway.

281. The stenotic incision tool of claim 280 wherein the holding member is adapted for use with a rapid exchange mechanism.

282. The stenotic incision tool of claim 280 further comprising the movable inflatable member.

283. The stenotic incision tool of claim 282 wherein the movable inflatable member comprises a plurality of balloons.

284. The stenotic incision tool of claim 283 wherein the movable inflatable member comprises a plurality of balloons comprising a single shaft.

285. The stenotic incision tool of claim 280 wherein the plurality of atherotomes is mounted in an expandable member.

286. The stenotic incision tool of claim 280 wherein the plurality of atherotomes is mounted in a single expandable member.

287. The stenotic incision tool of claim 280 wherein the plurality of atherotomes is mounted on more than one expandable member.

288. The stenotic incision tool of claim 280 further comprising a position control member configured to allow a user to place the tool at a selected site in the human body.

289. The stenotic incision tool of claim 288 where the position control member has a passageway substantially aligned with the inner substrate passageway, said passageway adapted to allow passage of the movable inflatable member to the passageway of the inner substrate.

290. The stenotic incision tool of claim 280 where outer member outer surface includes slits corresponding substantially to the positions of the atherotomes when the movable inflatable member is inflated in the passageway.

291. The stenotic incision tool of claim 280 where the plurality of atherotomes is exactly two mounted at approximately 180° to each other with respect to the atherotome holding member longitudinal axis.

292. The stenotic incision tool of claim 280 where the plurality of atherotomes is exactly four mounted at 90° to each other with respect to the atherotome holding member longitudinal axis.

293. The stenotic incision tool of claim 280 where the inner substrate extends to and comprises the outer member.

294. The stenotic incision tool of claim 280 where the inner substrate is spaced apart from the outer member.

295. The stenotic incision tool of claim 280 further comprising a catheter.

296. The stenotic incision tool of claim 280 further comprising a balloon catheter.

297. The stenotic incision tool of claim 280 further comprising a balloon catheter having more than one inflatable member.

298. The stenotic incision tool of claim 280 further comprising a balloon catheter having at least two inflatable members mounted radially adjacent each other.

299. The stenotic incision tool of claim 280 further comprising a balloon catheter having more than two inflatable members mounted radially adjacent each other.

300. The stenotic incision tool of claim 298 further comprising a balloon catheter having at least two inflatable members mounted longitudinally adjacent each other.

301. The stenotic incision tool of claim 280 further comprising a balloon catheter having at least two inflatable members mounted longitudinally adjacent each other.

302. The stenotic incision tool of claim 280 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

303. A shape control member for controllably limiting the expansion of an expandable member to a selected shape comprising:
a.) a fabric caul having a passageway configured for entry and exit of at least one expandable member, the caul being configured to limit the shape of at least one expandable member to a selected expanded shape when the at least one expandable member is expanded in the fabric caul passageway, and
b.) a position control member configured to allow a user to place the tool at a selected site in the human body when the caul has been inserted into the human body.

304. The shape control member of claim 303 adapted for use with a rapid exchange mechanism.

305. The shape control member of claim 303 further comprising at least two support members, and where the caul is mounted between a pair of the support members.

306. The shape control member of claim 303 where the position control member comprises a tubular member extending proximally and having a passageway substantially aligned with the fabric caul passageway, and said passageway adapted to allow passage of the expandable member.

307. The shape control member of claim 305 where the at least two support members are cylindrical.

308. The shape control member of claim 306 where the tubular member comprises a proximal support member.

309. The shape control member of claim 303 where the fabric caul has a substantially cylindrical expanded shape.

310. The shape control member of claim 309 where the substantially cylindrical expanded shape has a preselected diameter.

311. The shape control member of claim 303 comprising a plurality of fabric cauls having substantially cylindrical expanded shapes with preselected diameters.

312. The shape control member of claim 303 comprising a plurality of fabric cauls longitudinally separated from each other.

313. The shape control member of claim 303 wherein the plurality of fabric cauls are separated by and mounted between support members.

314. The shape control member of claim 311 where the preselected diameters are different.

315. The shape control member of claim 303 where the fabric caul has an expanded shape that is not cylindrical.

316. The shape control member of claim 303 where the caul, after expansion, has a tapering shape.

317. A combination drug delivery sleeve member and balloon catheter system for delivering a drug material to a body lumen, comprising:
a.) a drug carrier having a passageway configured for entry and exit of a balloon catheter having more than one inflatable member, the carrier being configured to allow release of a drug when at least one of the more than one inflatable member is inflated in the drug carrier passageway, and
b.) the balloon catheter comprising at least two inflatable members.

318. The combination drug delivery sleeve member and balloon catheter system of claim 317 adapted for use with a rapid exchange mechanism.

319. The combination drug delivery sleeve member and balloon catheter system of claim 317 wherein the balloon catheter comprises at least two inflatable members mounted radially adjacent each other.

320. The combination drug delivery sleeve member and balloon catheter system of claim 317 wherein the balloon catheter comprises more than two inflatable members mounted radially adjacent each other.

321. The combination drug delivery sleeve member and balloon catheter system of claim 317 wherein the balloon catheter comprises at least two inflatable members mounted longitudinally adjacent each other.

322. The combination drug delivery sleeve member and balloon catheter system of claim 321 wherein the balloon catheter further comprises at least two inflatable members mounted radially adjacent each other.

323. The combination drug delivery sleeve member and balloon catheter system of claim 317 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

324. The combination drug delivery sleeve member and balloon catheter system of claim 317 further comprising a guidewire.

325. The combination drug delivery sleeve member and balloon catheter system of claim 317 further comprising a position control member configured to allow a user to place the drug delivery member at a selected site in the human body.

326. The combination drug delivery sleeve member and balloon catheter system of claim 325 where the position control member comprises a tubular member extending proximally and having a passageway substantially aligned with the drug carrier passageway, and said passageway adapted to allow passage of the inflatable member.

327. The combination drug delivery sleeve member and balloon catheter system of claim 317 where the a drug carrier is configured to allow release of a drug when the inflatable member is inflated in the drug carrier passageway and causes the exterior wall to contact an interior of a body lumen.

328. The combination drug delivery sleeve member and balloon catheter system of claim 317 where the position control member comprises a proximal support member.

329. The combination drug delivery sleeve member and balloon catheter system of claim 317 where the drug carrier comprises a drug contained in a sleeve member having an exterior surface, and adapted to allow release of the drug to the exterior surface upon inflation of at least one of the at least two inflatable members.

330. The combination drug delivery sleeve member and balloon catheter system of claim 317 where the drug carrier comprises a drug contained in a sleeve member, a stenting implant for supporting the sleeve member, the drug carrier being configured to implant the drug-containing sleeve member and the stenting implant in the body lumen upon inflation of at least one of the at least two inflatable members.

331. The combination drug delivery sleeve member and balloon catheter system of claim 329 where the drug carrier further comprises an interior member configured to maintain a physical connection between the pair of adjacent support members after the drug-containing sleeve member and the stenting implant have been released in the body lumen after inflation of at least one of the two inflatable members.

332. The combination drug delivery sleeve member and balloon catheter system of claim 317 where the drug carrier comprises a drug contained in a sleeve member and where sleeve member is configured to implant the drug-containing sleeve member in the body lumen upon inflation of at least one of the two inflatable members.

333. The combination drug delivery sleeve member and balloon catheter system of claim 317 comprising a plurality of drug carriers.

334. The combination drug delivery sleeve member and balloon catheter system of claim 329 wherein the plurality of drug carriers are separated by and mounted between support members.

335. The combination drug delivery sleeve member and balloon catheter system of claim 317 where the drug carrier has a substantially constant diameter.

336. The combination drug delivery sleeve member and balloon catheter system of claim 317 where the drug carrier does not have a substantially constant diameter.

337. A component for controlling the longitudinal expansion of an inflatable member having a longitudinal axis, a proximal end, and a distal end, comprising:
a.) the inflatable member, and
b.) at least one expansion control member located adjacent one of the inflatable member distal or proximal ends, having an axis generally coincident with the longitudinal axis of the inflatable member, an expansion end adjacent the inflatable member, and a second end more remote from the inflatable member than the expansion end, the at least one expansion control member having a stiffness sufficient to allow, as a result of inflatable member expansion, the expansion end to expand in an amount greater than the expansion of the second end, and to direct the expansion of the inflatable member away from the expansion end.

338. The longitudinal expansion control component of claim 337 wherein the inflatable member comprises an elastic material.

339. The longitudinal expansion control component of claim 337 wherein the inflatable member comprises an inelastic material.

340. The longitudinal expansion control component of claim 337 wherein the inflatable member comprises at least one of two inflatable members mounted radially adjacent each other.

341. The longitudinal expansion control component of claim 337 wherein the inflatable member comprises at least one of more than two inflatable members mounted radially adjacent each other.

342. The longitudinal expansion control component of claim 337 wherein the inflatable member comprises at least one of two inflatable members mounted longitudinally adjacent each other.

343. The longitudinal expansion control component of claim 342 wherein the inflatable member comprises at least one of at least two inflatable members mounted radially adjacent each other.

344. The longitudinal expansion control component of claim 337 wherein the inflatable member comprises a core guide inflatable member mounted on a core guide member where the core guide member comprises at least one inflation area, a proximal end, a distal end, a core passageway that is fluidly connected with the at least one inflation area, and is closable distally of the at least one inflation area; having said at least one core guide inflatable member with a length, the core guide inflatable member surrounding at least a portion of one of the at least one inflation area, and that is sealingly connected to the core guide member to form an inflatable region in fluid connection with the core passageway.

345. The longitudinal expansion control component of claim 337 wherein the inflatable member comprises at least one inflatable member of a balloon catheter having at least one inflatable member.

346. The longitudinal expansion control component of claim 345 further comprising a guidewire.

347. The longitudinal expansion control component of claim 337 comprising exactly two expansion control members, each one located adjacent one of the elastic inflatable member proximal and distal ends.

348. The longitudinal expansion control component of claim 337 wherein one of the at least one expansion control members is integral with one of the inflatable member proximal and distal ends.

349. The longitudinal expansion control component of claim 348 wherein one of the at least one expansion control members is integral with the inflatable member distal end.

350. The longitudinal expansion control component of claim 348 wherein one of the at least one expansion control members is integral with the inflatable member proximal end.

351. The longitudinal expansion control component of claim 337 wherein one of the at least one expansion control members is slidable over one of the inflatable member proximal and distal ends.

352. The longitudinal expansion control component of claim 351 wherein one of the at least one expansion control members is slidable over the inflatable member proximal end.

353. The longitudinal expansion control component of claim 352 wherein the at least one slidable expansion control member comprises a constraining member configured to constrain inflation of the inflatable member proximally of the expansion control member expansion end, and to permit inflation of the inflatable member distally of the expansion control member expansion end.

354. The longitudinal expansion control component of claim 353 wherein the at least one slidable expansion control member is fixedly attached to a proximally extending position control member.

355. The longitudinal expansion control component of claim 354 wherein the proximally extending position control member is tubular.

356. The longitudinal expansion control component of claim 354 wherein the at least one slidable expansion control member comprises a material that has a flexural stiffness higher than the flexural stiffness of the material comprising the inflatable member.

357. The longitudinal expansion control component of claim 356 wherein the at least one slidable expansion control member comprises a material that has a flexural stiffness lower than the flexural stiffness of the material comprising the member extending proximally.

358. The longitudinal expansion control component of claim 337 wherein the at least one expansion control member comprises a material that has a flexural stiffness higher than the flexural stiffness of the material comprising the inflatable member.

359. The longitudinal expansion control component of claim 337 wherein the at least one expansion control member comprises one or more longitudinal stiffeners.

360. The longitudinal expansion control component of claim 337 wherein the at least one expansion control member comprises at least one convoluted limiter ring configured to de-convolute upon expansion and to limit the expansion of the expander end to a determined limit when de-convoluted.

361. The longitudinal expansion control component of claim 360 wherein the at least one convoluted limiter ring is situated between the expander end and the second end.

362. The longitudinal expansion control component of claim 337 wherein the at least one expansion control member comprises at least one cinch ring adjacent the second end configured to substantially prevent the expansion of the second end.

363. The longitudinal expansion control component of claim 337 wherein the at least one expansion control member comprises a plurality of closed slots in the at least one expansion control member configured to allow and to limit the expansion of at least a portion of the expander end to a predetermined limit.

364. A sterilized medical device system kit comprising:
a sterilized sealed packaging containing:
a medical device system having at least an adjustable-length inflatable member comprising:
a core guide member having at least one inflation area, a proximal end, a distal end, a core passageway that is fluidly connected with the at least one inflation area, and is closable distally of the at least one inflation area; having at least one inflatable member having a length, surrounding at least a portion of the inflation area, and that is sealingly connected to the core guide member to form an inflatable region in fluid connection with the passageway; and
at least one constraining member longitudinally slidable along the core guide member, having a distal end, and wherein the constraining member is configured to slide upon the at least inflatable member, to constrain inflation of the at least one inflatable member proximally of the constraining member distal end, and to permit inflation of the at least one inflatable member distally of the constraining member distal end, whereby the longitudinal movement of the constraining member adjusts the length of the at least one inflatable member available for inflation.

365. The sterilized medical device system kit of claim 364 further comprising at least one stenting device implantable from the medical device system.

366. The sterilized medical device system kit of claim 364 further comprising at least one stenting device delivery sleeve having an interior longitudinal opening, a distal end, a proximal end, and of a size, flexibility, and material appropriate to support stenting devices, and to allow deployment of those stenting devices independently without substantially affecting adjacent stenting devices, and at least one stenting device implantable from the medical device system.

367. The sterilized medical device system kit of claim 364 where the at least one stenting device delivery sleeve is filamentary and is further configured to deploy those stenting devices independently by inflating at least an inflatable member in the interior longitudinal opening and to return to a pre-deployment shape without substantial plastic deformation.

368. The sterilized medical device system kit of claim 366 wherein the at least one stenting device delivery sleeve has a substantially constant diameter.

369. The sterilized medical device system kit of claim 366 wherein the at least one stenting device delivery sleeve does not have a substantially constant diameter.

370. The sterilized medical device system kit of claim 364 further comprising at least one elastic sleeve having an interior longitudinal opening, a distal end, a proximal end, and of a size, flexibility, and material appropriate to support stenting devices, to allow deployment of those stenting devices independently without substantially affecting adjacent stenting devices, an elongate position control member attached to a proximal end of the sleeve, and at least one stenting device.

371. The sterilized medical device system kit of claim 370 where the at least one elastic sleeve is further configured to deploy those stenting devices independently by inflating a inflatable member in the interior longitudinal opening and to return to a pre-deployment shape without substantial plastic deformation.

372. The sterilized medical device system kit of claim 370 where the at least one stenting device is detachably mounted exterior to the sleeve member.

373. The sterilized medical device system kit of claim 366 comprising more than one stenting device, at least one being deployable using a inflatable member.

374. The sterilized medical device system kit of claim 366 comprising more than one stenting device, at least one being self-expanding.

375. The sterilized medical device system kit of claim 373 further comprising a removable retainer configured to controllably allow the more than one self-expanding stenting devices to individually self-deploy.

376. The sterilized medical device system kit of claim 366 further comprising: a shape control member for controllably limiting the expansion of the inflatable member to a selected shape comprising:
a.) at least one fabric caul having a passageway configured for entry and exit of a removable, expandable member, the caul being configured to limit the shape of the inflatable member to a selected expanded shape when the expandable member is inflated in the fabric caul passageway, and
b.) position control member configured to allow a user to place the fabric caul member at a selected site in the human body.

377. The sterilized medical device system kit of claim 376 wherein the shape control member further comprises at least two support members.

378. The sterilized medical device system kit of claim 376 where the position control member comprises a tubular member extending proximally from the at least one caul and having a passageway substantially aligned with the fabric caul passageway, and said passageway adapted to allow passage of the at least one movable, expandable member.

379. The sterilized medical device system kit of claim 378 where the fabric caul has a substantially cylindrical expanded shape.

380. The sterilized medical device system kit of claim 378 where the substantially cylindrical expanded shape has a preselected diameter.

381. The sterilized medical device system kit of claim 376 comprising a plurality of fabric cauls having substantially cylindrical expanded shapes with preselected diameters.

382. The sterilized medical device system kit of claim 381 wherein the plurality of fabric cauls are separated by and mounted between support members.

383. The sterilized medical device system kit of claim 381 where the preselected diameters are different.

384. The sterilized medical device system kit of claim 381 where the fabric caul has an expanded shape that is not cylindrical.

385. The sterilized medical device system kit of claim 364 further comprising at least one expansion control member located adjacent one of the inflatable member distal or proximal ends, having an axis generally coincident with the longitudinal axis of the inflatable member, an expansion end adjacent the inflatable member, and a second end more remote from the inflatable member than the expansion end, the at least one expansion control member having a stiffness sufficient to allow, during inflation of the inflatable member, the expansion end to expand in an amount greater than the expansion of the second end, and to direct the expansion of the inflatable member away from the first end.

386. The sterilized medical device system kit of claim 385 comprising exactly two expansion control members, each one located at one of the at least one elastic inflatable member proximal and a distal ends.

387. The sterilized medical device system kit of claim 385 wherein one of the at least one expansion control members is integral with one of the at least one inflatable member proximal and distal ends.

388. The sterilized medical device system kit of claim 385 wherein one of the at least one expansion control members is integral with the at least one inflatable member distal end.

389. The sterilized medical device system kit of claim 385 wherein one of the at least one expansion control members is integral with the at least one inflatable member proximal end.

390. The sterilized medical device system kit of claim 385 wherein one of the at least one expansion control members is slidable over one of the at least one inflatable member proximal and distal ends.

391. The sterilized medical device system kit of claim 385 wherein one of the at least one expansion control members is slidable over the at least one inflatable member proximal end.

392. The sterilized medical device system kit of claim 390 wherein the at least one slidable expansion control member comprises a constraining member configured to constrain inflation of the inflatable member proximally of the expansion control member expansion end, and to permit inflation of the inflatable member distally of the expansion control member expansion end.

393. The sterilized medical device system kit of claim 391 wherein the at least one slidable expansion control member is fixedly attached to a tubing member extending proximally.

394. The sterilized medical device system kit of claim 391 wherein the at least one slidable expansion control member comprises a material that has a flexural stiffness higher than the flexural stiffness of the material comprising the inflatable member.

395. The sterilized medical device system kit of claim 391 wherein the at least one slidable expansion control member comprises a material that has a flexural stiffness lower than the flexural stiffness of the material comprising the tubing member extending proximally.

396. The sterilized medical device system kit of claim 391 wherein the at least one expansion control member comprises a material that has a flexural stiffness higher than the flexural stiffness of the material comprising the inflatable member.

397. The sterilized medical device system kit of claim 391 wherein the at least one expansion control member comprises one or more longitudinal stiffeners.

398. The sterilized medical device system kit of claim 391 wherein the at least one expansion control member comprises at least one limiter ring adjacent the expander end configured to limit the expansion of the expander end to a determined limit.

399. The sterilized medical device system kit of claim 391 wherein the at least one expansion control member comprises at least one cinch ring adjacent the second end configured to substantially prevent the expansion of the second end.

400. The sterilized medical device system kit of claim 391 wherein the at least one expansion control member comprises at least one convoluted ring adjacent the second end configured to substantially prevent the expansion of the second end.

401. The sterilized medical device system kit of claim 391 wherein the at least one expansion control member comprises a plurality of closed slots in the at least one expansion control member configured to allow and to limit the expansion of the expander end to a determined limit.

402. The sterilized medical device system kit of claim 364 further comprising a balloon catheter.

403. The sterilized medical device system kit of claim 364 further comprising a balloon catheter containing multiple inflatable members.

404. The sterilized medical device system kit of claim 364 adapted for use with a rapid exchange mechanism.

405. The sterilized medical device system kit of claim 364 further comprising a balloon catheter comprising at least one atherotome configured to radially extend from the balloon catheter upon expansion of the catheter.

406. The sterilized medical device system kit of claim 365 further comprising an inelastic balloon configured to expand and to deploy the stent.

407. The sterilized medical device system kit of claim 365 further comprising an elastic balloon configured to expand and to deploy the stent.

408. A sterilized stent delivery sleeve kit comprising:
sterilized sealed packaging containing:
at least one filamentary sleeve having an interior longitudinal opening and wherein the filaments are of a size, flexibility, and shape and comprising materials appropriate a.) to support stenting devices and b.) to deploy those stenting devices independently without substantially affecting adjacent stents and
at least one stenting device mountable exterior to the at least one filamentary sleeve.

409. The sterilized stent delivery sleeve kit of claim 408 further comprising a position control member configured to allow a user to place the tool at a selected site in the human body.

410. The sterilized stent delivery sleeve kit of claim 408 where the filamentary sleeve further is configured to deploy those stenting devices independently by inflating a inflatable member in the interior longitudinal opening and to return to a pre-deployment shape without substantial plastic deformation.

411. The sterilized stent delivery sleeve kit of claim 408 where the at least one stenting device comprises more than one stenting device of which at least one is deployable using an inflatable member.

412. The sterilized stent delivery sleeve kit of claim 408 where the at least one stenting device of which at least one is a self-expanding stenting device.

413. The sterilized stent delivery sleeve kit of claim 412 further comprising a movable retainer configured to controllably allow the at least one self-expanding stenting device to individually self-deploy.

414. The sterilized stent delivery sleeve kit of claim 408 comprising more than one filamentary sleeve each having at least one stenting device joinable to another filamentary sleeve having at least one stenting device.

415. The sterilized stent delivery sleeve kit of claim 408 comprising more than one filamentary sleeve having at least one stenting device joinable to another filamentary sleeve having at least one stenting device and further joined to an elongate position control member attached to an end of one of the filamentary sleeves.

416. The sterilized medical device system kit of claim 408 wherein the at least one filamentary sleeve has a substantially constant diameter.

417. The sterilized medical device system kit of claim 408 wherein the at least one filamentary sleeve does not have a substantially constant diameter.

418. The sterilized stent delivery sleeve kit of claim 408 comprising more than one stenting device each having substantially the same length.

419. The sterilized stent delivery sleeve kit of claim 408 comprising more than one stenting device wherein they do not have the same length.

420. The sterilized stent delivery sleeve kit of claim 408 comprising an elongate position control member joinable to an end of a filamentary sleeve.

421. The sterilized stent delivery sleeve kit of claim 408 where the at least one stenting device further comprises at least one biologically active agent.

422. The sterilized stent delivery sleeve kit of claim 408 where the at least one stenting device further comprises a releasable biologically active agent selected from the group consisting of anti-proliferation agents, anti-inflammatory agents, antibiotics, and immunosuppressants.

423. The sterilized stent delivery sleeve kit of claim 408 where the at least one stenting device further comprises a releasable biologically active agent selected from the group consisting of paclitaxel, prednisolone, methotrexate, batimastal, doxycycline, tetracycline, rapamycin, actinomycin, dexamethosone, methyl prednisolone, nitroprussides, estrogen, estradiols, and their mixtures.

424. The sterilized stent delivery sleeve kit of claim 410 further comprising a balloon catheter or core guide member comprising the inflatable member.

425. The sterilized stent delivery sleeve kit of claim 410 further comprising a balloon catheter comprising multiple inflatable members or core guide member comprising multiple inflatable members.

426. The sterilized stent delivery sleeve kit of claim 408 adapted for use with a rapid exchange mechanism.

427. A combination stent delivery sleeve and balloon catheter medical device system comprising:
a balloon catheter having more than one inflatable member,
at least one elastic sleeve member having an interior longitudinal opening, a distal end, a proximal end, and of a size, flexibility, and material appropriate to support stenting devices, to allow deployment of those stenting devices independently without substantially affecting adjacent stenting devices;
at least one stenting device detachably mounted exterior to the sleeve member.

428. The combination medical device system of claim 427 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

429. The combination medical device system of claim 427 further comprising a position control member configured to allow a user to direct the position of the sleeve from exterior of the body.

430. The combination medical device system of claim 427 wherein the at least one elastic sleeve has a substantially constant diameter.

431. The combination medical device system of claim 427 wherein the at least one elastic sleeve does not have a substantially constant diameter.

432. The combination medical device system of claim 427 wherein at least one of the stenting devices is deployable using an inflatable member.

433. The combination medical device system of claim 427 wherein at least one of the stenting devices is self-expanding.

434. The combination medical device system of claim 433 further comprising a removable retainer configured to controllably allow the self-expanding stenting devices to individually self-deploy.

435. The combination medical device system of claim 427 where the stenting devices comprise stenting devices each having substantially the same length.

436. The combination medical device system of claim 427 where at least one of the stenting devices further comprises at least one biologically active agent.

437. The sterilized stent delivery sleeve kit of claim 427 where the at least one stenting device further comprises a releasable biologically active agent selected from the group consisting of paclitaxel, prednisolone, methotrexate, batimastal, doxycycline, tetracycline, rapamycin, actinomycin, dexamethosone, methyl prednisolone, nitroprussides, estrogen, estradiols, and their mixtures.

438. The combination medical device system of claim 427 adapted for use with a rapid exchange mechanism.

439. The combination medical device system of claim 427 further comprising an inelastic balloon configured to expand and to deploy the stent.

440. The combination medical device system of claim 427 further comprising an elastic balloon configured to expand and to deploy the stent.

441. The combination medical device system of claim 427 wherein the balloon catheter comprises at least two inflatable members mounted radially adjacent each other.

442. The combination medical device system of claim 427 wherein the balloon catheter comprises more than two inflatable members mounted radially adjacent each other.

443. The combination medical device system of claim 427 wherein the balloon catheter comprises at least two inflatable members mounted longitudinally adjacent each other.

444. The combination medical device system of claim 443 wherein the balloon catheter further comprises at least two inflatable members mounted radially adjacent each other.

445. A sterilized stent delivery sleeve kit comprising:
sterilized sealed packaging containing:
a stent delivery sleeve comprising:
at least one elastic sleeve having an interior longitudinal opening, a distal end, a proximal end, and of a size, flexibility, and material appropriate to support stenting devices, to allow deployment of those stenting devices independently without substantially affecting adjacent stenting devices; and
more than one stenting device detachably movable exterior to the sleeve member.

446. The sterilized stent delivery sleeve kit of claim 445 further comprising a position control member configured to allow a user to place the tool at a selected site in the human body.

447. The sterilized stent delivery sleeve kit of claim 445 where the elastic sleeve further is configured to deploy those stenting devices independently by inflating a inflatable member in the interior longitudinal opening and to return to a pre-deployment shape without substantial plastic deformation.

448. The sterilized stent delivery sleeve kit of claim 445 where the at least one stenting device comprises more than one stenting device of which at least one is deployable using an inflatable member.

449. The sterilized stent delivery sleeve kit of claim 445 where the at least one stenting device of which at least one is a self-expanding stenting device.

450. The sterilized stent delivery sleeve kit of claim 449 further comprising a removable retainer configured to controllably allow the at least one self-expanding stenting device to individually self-deploy.

451. The sterilized stent delivery sleeve kit of claim 445 comprising more than one elastic sleeve each having at least one stenting device joinable to another elastic sleeve having at least one stenting device.

452. The sterilized stent delivery sleeve kit of claim 445 comprising more than one elastic sleeve having at least one stenting device joinable to another elastic sleeve having at least one stenting device and further joined to an elongate position control member attached to an end of one of the elastic sleeves.

453. The sterilized medical device system kit of claim 445 wherein the at least one elastic sleeve has a substantially constant diameter.

454. The sterilized medical device system kit of claim 445 wherein the at least one elastic sleeve does not have a substantially constant diameter.

455. The sterilized stent delivery sleeve kit of claim 445 comprising more than one stenting device each having substantially the same length.

456. The sterilized stent delivery sleeve kit of claim 445 where the at least one stenting device further comprises at least one biologically active agent.

457. The sterilized stent delivery sleeve kit of claim 445 where the at least one stenting device further comprises a releasable biologically active agent selected from the group consisting of anti-proliferation agents, anti-inflammatory agents, antibiotics, and immunosuppressants.

458. The sterilized stent delivery sleeve kit of claim 445 where the at least one stenting device further comprises a releasable biologically active agent selected from the group consisting of paclitaxel, prednisolone, methotrexate, batimastal, doxycycline, tetracycline, rapamycin, actinomycin, dexamethosone, methyl prednisolone, nitroprussides, estrogen, estradiols, and their mixtures.

459. The sterilized stent delivery sleeve kit of claim 445 further comprising a balloon catheter.

460. The sterilized stent delivery sleeve kit of claim 445 further comprising a balloon catheter containing multiple inflatable members.

461. The sterilized stent delivery sleeve kit of claim 445 adapted for use with a rapid exchange mechanism.

462. A method for adjusting the length or diameter of an inflatable member in a medical device system comprising the steps of:
a.) providing the device of claim 157,
b.) placing the inflatable member at a selected site,
c.) sliding a constraining member along the core guide member on the proximal end of the inflatable member until a selected inflatable member length is achieved, and
d.) inflating the inflatable member.

463. The process of claim 462 further comprising the step of deflating the inflatable member.

464. The process of claim 463 further comprising the step of moving the deflated inflatable member to another site in the human body, adjusting the size of the inflatable member by moving the constraining member to a second selected inflatable member size, and inflating inflatable member.

465. The process of claim 464 further comprising the step of deflating the inflatable member.

466. A procedure for adjusting the length of an inflatable member in a medical device system comprising the steps of:
a.) providing the device of claim 157,
b.) placing the inflatable member at a selected site in the human body,
c.) sliding a constraining member along the core guide member on the proximal end of the inflatable member until a selected inflatable member length is achieved,
d.) sliding a stent delivery sleeve having at least one stenting device on its exterior to the selected site; and
e.) inflating inflatable member to implant the stenting device.

467. The procedure of claim 466 further comprising the step of inflating the member to reform the stenting device.

468. The procedure of claim 466 further comprising the steps of:
a.) deflating the inflatable member,
b.) proximally withdrawing the stent delivery sleeve from the selected site,
c.) positioning the inflatable member at a selected portion of the implanted stent;
d.) selecting the size of the inflatable member by moving the constraining member,
e.) inflating the inflatable member to reform the shape of the implanted stenting device, and
f.) deflating the inflatable member.

469. The procedure of claim 466 further comprising the step of deflating the inflatable member.

470. The procedure of claim 466 further comprising the steps of:
a.) deflating the inflatable member,
b.) placing the inflatable member at a second selected site in the human body,
c.) sliding a constraining member along the core guide member on the proximal end of the inflatable member until a selected inflatable member length is achieved,
d.) sliding the stent delivery sleeve having at least one stenting device on its exterior to the selected site; and
e.) inflating inflatable member to implant the stenting device.

471. The procedure of claim 470 further comprising the step of deflating the inflatable member.

472. A medical device system having more than one inflatable member comprising:
a balloon catheter having more than one inflatable member; and
at least one constraining member longitudinally slidable along the balloon catheter, having a distal end, and wherein the constraining member is configured to be remotely slidable by a user after the balloon catheter and constraining member are introduced in the body, to slide upon the inflatable member, to constrain inflation of the inflatable member proximally of the constraining member distal end, and to permit inflation of the inflatable member distally of the constraining member distal end.

473. The medical device system of claim 472 wherein the longitudinal movement of the constraining member adjusts the length of the inflatable member available for inflation.

474. The medical device system of claim 472 where at least one of the inflatable members comprises elastomeric material.

475. The medical device system of claim 472 where at least one of the inflatable members comprises non-elastomeric material.

476. The medical device system of claim 472 where at least two of the inflatable members are mounted radially adjacent each other.

477. The medical device system of claim 472 where more than two of the inflatable members are mounted radially adjacent each other.

478. The medical device system of claim 472 where at least two of the inflatable members are mounted longitudinally adjacent each other.

479. The medical device system of claim 478 where at least two of the inflatable members are mounted radially adjacent each other.

480. The medical device system of claim 472 further comprising a comparatively lower profile distal inflatable member section

481. The medical device system of claim 472 where each of the inflatable members is independently inflatable.

482. The medical device system of claim 472 where each of the inflatable members is inflatable through a single lumen.

483. The medical device system of claim 472 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

484. The medical device system of claim 483 further comprising at least one stenting structure and the at least one stenting structure is in contact with at least one of the core guide inflatable members.

485. The medical device system of claim 472 further comprising a guidewire.

486. The medical device system of claim 485 further comprising at least one stenting structure and the at least one stenting structure is in contact with at least one of the balloon catheter inflatable members.

487. The medical device system of claim 480 where the distal inflatable member section is a compliant balloon.

488. The medical device system of claim 480 where the distal inflatable member section is a semi-compliant balloon.

489. The medical device system of claim 480 where the distal inflatable member section is a non-compliant balloon.

490. The medical device system of claim 472 further comprising an outer elastic member radially adjacent to at least one of the more than one of inflatable members, configured to restore the at least one inflatable member to a lower profile upon deflation of the inflatable member.

491. The medical device system of claim 472 further comprising an outer elastic member radially adjacent to at least one of the more than one of inflatable members, to push inflation fluid out of said inflatable members.

492. The medical device system of claim 472 further comprising a guide catheter.

493. The medical device system of claim 472 further comprising at least one stenting structure.

494. The medical device system of claim 493 wherein the at least one stenting structure is in contact with at least one of the inflatable members.

495. The medical device system of claim 472 further comprising a plurality of stenting structures.

496. The medical device system of claim 495 wherein the plurality of stenting structures is in contact with at least one of the inflatable members.

497. The medical device system of claim 472 further comprising a plurality of stenting structures mounted upon a stent delivery sleeve and wherein the stent delivery sleeve comprises at least one sleeve having an interior longitudinal opening and wherein the sleeve is configured to deploy those stenting devices independently without substantially affecting adjacent stenting devices.

498. The medical device system of claim 497 wherein the stent delivery sleeve is slidable upon the at least one constraining member.

499. The medical device system of claim 497 wherein the stent delivery sleeve is slidable beneath the at least one constraining member.

500. The medical device system of claim 497 wherein the sleeve is configured to deploy at least one of those stenting devices independently by inflating an inflatable member in the interior longitudinal opening.

501. The medical device system of claim 472 wherein at least one inflatable member has a profile selected from the group consisting of columnar, tapered, stepped and combinations thereof.

502. The medical device system of claim 472 wherein each of the at least one inflatable members is independently inflatable by a separate inflation fluid tubing member.

503. The medical device system of claim 472 wherein each of the at least one inflatable members is independently inflatable by a separate inflation fluid passageway.

504. The medical device system of claim 472 wherein each of the at least one inflatable members is inflatable from a common inflation fluid passageway.

505. The medical device system of claim 472 wherein the balloon catheter has a catheter wall adapted to pass a rapid exchange wire

506. The medical device system of claim 472 wherein the balloon catheter is adapted to permit use of a rapid exchange system.

507. The medical device system of claim 472 wherein the balloon catheter comprises a catheter wall adapted to allow passage of a rapid exchange wire through the wall proximally of the more than one inflatable member.

508. The medical device system of claim 507 further comprising one or more rapid exchange wires.

509. A medical device system having more than one inflatable member comprising:
a.) a balloon catheter having more than one inflatable member; and
b.) at least one stenting structure in contact with at least one of the inflatable members,
wherein at least one inflatable member is configured to expand at least one stenting structure.

510. The medical device system of claim 509 further comprising a plurality of stenting structures.

511. The medical device system of claim 510 wherein the plurality of stenting structures is in contact with at least one of the inflatable members.

512. The medical device system of claim 510 further comprising a sleeve mounted to cover at least one of the plurality of stenting structures in contact with at least one of the inflatable members and wherein the sleeve is configured to be substantially fixed after placement of the balloon catheter in the body and to prevent expansion of the covered at least one stenting structure.

513. The medical device system of claim 509 further comprising at least one constraining member longitudinally slidable along the balloon catheter, having a distal end, and wherein the constraining member is configured to be remotely slidable by a user after the balloon catheter and constraining member are introduced in the body, to slide upon the inflatable member, to constrain inflation of the inflatable member proximally of the constraining member distal end, and to permit inflation of the inflatable member distally of the constraining member distal end.

514. The medical device system of claim 513 wherein the longitudinal movement of the constraining member adjusts the length of the inflatable member available for inflation.

515. The medical device system of claim 513 wherein the constraining member is adapted to slide beneath the stenting structure.

516. The medical device system of claim 513 wherein the constraining member is adapted to slide above the stenting structure.

517. The medical device system of claim 509 where at least one of the inflatable members comprises elastomeric material.

518. The medical device system of claim 509 where at least of the inflatable members comprises non-elastomeric material.

519. The medical device system of claim 509 where at least two of the inflatable members are mounted radially adjacent each other.

520. The medical device system of claim 509 where more than two of the inflatable members are mounted radially adjacent each other.

521. The medical device system of claim 509 where at least two of the inflatable members are mounted longitudinally adjacent each other.

522. The medical device system of claim 521 where at least two of the inflatable members are mounted radially adjacent each other.

523. The medical device system of claim 509 where each of the inflatable members is independently inflatable.

524. The medical device system of claim 509 where each of the inflatable members is inflatable through a single lumen.

525. The medical device system of claim 509 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

526. The medical device system of claim 525 further comprising at least one stenting structure and the at least one stenting structure is in contact with at the at least one core guide inflatable member.

527. The medical device system of claim 509 further comprising a comparatively lower profile distal inflatable member section.

528. The medical device system of claim 527 where the distal inflatable member section comprises a compliant balloon.

529. The medical device system of claim 527 where the distal inflatable member section comprises a semi-compliant balloon.

530. The medical device system of claim 527 where the distal inflatable member section comprises a non-compliant balloon.

531. The medical device system of claim 509 further comprising an outer elastic sleeve radially adjacent to at least one of the more than one of inflatable members, configured to restore the at least one inflatable member to a lower profile upon deflation of the inflatable member.

532. The medical device system of claim 509 further comprising an outer elastic sleeve radially adjacent to at least one of the more than one of inflatable members, to push inflation fluid out of said inflatable members.

533. The medical device system of claim 509 further comprising a guide catheter.

534. The medical device system of claim 509 wherein at leas one inflatable member has a profile selected from the group consisting of columnar, tapered, stepped and combinations thereof.

535. The medical device system of claim 509 wherein the balloon catheter is adapted for a rapid exchange system.

536. The medical device system of claim 509 wherein the balloon catheter has a catheter wall adapted to pass a rapid exchange wire.

537. The medical device system of claim 509 wherein the balloon catheter has a catheter wall adapted to allow passage of a rapid exchange wire through the catheter wall proximally of the more than one inflatable member.

538. The medical device system of claim 513 wherein the balloon catheter has a catheter wall adapted to allow passage of a rapid exchange wire through the catheter wall proximally of the more than one inflatable member and the constraining member also comprises a wall adapted to pass a rapid exchange wire.

539. The medical device system of claim 509 comprising exactly one guidewire and that guidewire is passable through the catheter wall and the constraining member wall.

540. The medical device system of claim 538 further comprising one or more rapid exchange wires.

541. The medical device system of claim 509 further comprising a guide wire.

542. A medical device system having more than one inflatable member comprising:
a.) a core guide member having more than one inflatable member; and
b.) at least one stenting structure in contact with at least one of the inflatable members,
wherein at least one inflatable member is configured to expand at least one stenting structure.

543. The medical device system of claim 542 further comprising a plurality of stenting structures.

544. The medical device system of claim 543 wherein the plurality of stenting structures is in contact with at least one of the inflatable members.

545. The medical device system of claim 543 further comprising a sleeve mounted to cover at least one of the plurality of stenting structures in contact with at least one of the inflatable members and wherein the sleeve is configured to be substantially fixed with respect to the core guide member after placement of the core guide member in the body and to prevent expansion of the covered at least one stenting structure.

546. The medical device system of claim 542 further comprising at least one constraining member longitudinally slidable along the core guide member, having a distal end, and wherein the constraining member is configured to be remotely slidable by a user after the core guide member and constraining member are introduced in the body, to slide upon the inflatable member, to constrain inflation of the inflatable member proximally of the constraining member distal end, and to permit inflation of the inflatable member distally of the constraining member distal end.

547. The medical device system of claim 546 wherein the longitudinal movement of the constraining member adjusts the length of the inflatable member available for inflation.

548. The medical device system of claim 546 wherein the constraining member is adapted to slide beneath the stenting structure.

549. The medical device system of claim 546 wherein the constraining member is adapted to slide above the stenting structure.

550. The medical device system of claim 542 where at least one of the inflatable members comprises elastomeric material.

551. The medical device system of claim 542 where at least of the inflatable members comprises non-elastomeric material.

552. The medical device system of claim 542 where at least two of the inflatable members are mounted radially adjacent each other.

553. The medical device system of claim 542 where more than two of the inflatable members are mounted radially adjacent each other.

554. The medical device system of claim 542 where at least two of the inflatable members are mounted longitudinally adjacent each other.

555. The medical device system of claim 554 where at least two of the inflatable members are mounted radially adjacent each other.

556. The medical device system of claim 542 where each of the inflatable members is independently inflatable.

557. The medical device system of claim 542 where each of the inflatable members is inflatable through a single lumen.

558. The medical device system of claim 542 further comprising a comparatively lower profile distal inflatable member section.

559. The medical device system of claim 558 where the distal inflatable member section is a compliant balloon.

560. The medical device system of claim 558 where the distal inflatable member section is a semi-compliant balloon.

561. The medical device system of claim 558 where the distal inflatable member section is a non-compliant balloon.

562. The medical device system of claim 542 further comprising an outer elastic sleeve radially adjacent to at least one of the more than one of inflatable members, configured to restore the at least one inflatable member to a lower profile upon deflation of the inflatable member.

563. The medical device system of claim 542 further comprising an outer elastic sleeve radially adjacent to at least one of the more than one of inflatable members, to push inflation fluid out of said inflatable members.

564. The medical device system of claim 542 further comprising a guide catheter.

565. The medical device system of claim 542 wherein at least one inflatable member has a profile selected from the group consisting of columnar, tapered, stepped and combinations thereof.

566. A medical device system having more than one inflatable member comprising a balloon catheter having more than one inflatable member; and wherein the balloon catheter is adapted for a rapid wire mechanism.

567. The medical device system of claim 566 wherein the balloon catheter has a catheter wall adapted to pass a rapid exchange wire.

568. The medical device system of claim 566 wherein the balloon catheter has a catheter wall adapted for passage of a rapid exchange wire through the wall proximally of the more than one inflatable member.

569. The medical device system of claim 566 wherein the balloon catheter has a catheter wall adapted for passage of a rapid exchange wire through the wall distally of the more than one inflatable member.

570. The medical device system of claim 568 further comprising one or more rapid exchange wires.

571. The medical device system of claim 566 further comprising at least one stenting structure in contact with at least one of the inflatable members,

572. The medical device system of claim 566 wherein at least one inflatable member is configured to expand at least one stenting structure.

573. The medical device system of claim 566 further comprising a plurality of stenting structures.

574. The medical device system of claim 572 wherein the plurality of stenting structures is in contact with at least one of the inflatable members.

575. The medical device system of claim 566 further comprising at least one constraining member longitudinally slidable along the balloon catheter, having a distal end, and wherein the constraining member is configured to be remotely slidable by a user after the balloon catheter and constraining member are introduced in the body, to slide upon the inflatable member, to constrain inflation of the inflatable member proximally of the constraining member distal end, and to permit inflation of the inflatable member distally of the constraining member distal end.

576. The medical device system of claim 575 wherein the longitudinal movement of the constraining member adjusts the length of the inflatable member available for inflation.

577. The medical device system of claim 575 where at least one of the inflatable members comprises elastomeric material.

578. The medical device system of claim 566 where at least of the inflatable members comprises non-elastomeric material.

579. The medical device system of claim 566 where at least two of the inflatable members are mounted radially adjacent each other.

580. The medical device system of claim 566 where more than two of the inflatable members are mounted radially adjacent each other.

581. The medical device system of claim 566 where at least two of the inflatable members are mounted longitudinally adjacent each other.

582. The medical device system of claim 581 where at least two of the inflatable members are mounted radially adjacent each other.

583. The medical device system of claim 566 where each of the inflatable members is independently inflatable.

584. The medical device system of claim 566 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

585. The medical device system of claim 584 further comprising at least one stenting structure and the at least one stenting structure is in contact with at least one of the core guide inflatable members.

586. The medical device system of claim 584 further comprising a comparatively lower profile distal inflatable member section.

587. The medical device system of claim 584 where the distal inflatable member section is a compliant balloon.

588. The medical device system of claim 584 where the distal inflatable member section is a semi-compliant balloon.

589. The medical device system of claim 584 where the distal inflatable member section is a non-compliant balloon.

590. The medical device system of claim 566 further comprising an outer elastic sleeve radially adjacent to at least one of the more than one of inflatable members, configured to restore the at least one inflatable member to a lower profile upon deflation of the inflatable member.

591. The medical device system of claim 566 further comprising an outer elastic sleeve radially adjacent to at least one of the more than one of inflatable members, to push inflation fluid out of said inflatable members.

592. The medical device system of claim 566 further comprising a guide catheter.

593. The medical device system of claim 566 wherein at least one inflatable member has a profile selected from the group consisting of columnar, tapered, stepped and combinations thereof.

594. The medical device system of claim 566 wherein each of the at least one inflatable members is independently inflatable by a separate inflation fluid tubing member.

595. The medical device system of claim 566 wherein each of the at least one inflatable members is inflatable from a common inflation fluid passageway.

596. A medical device system having more than one inflatable member comprising:
a balloon catheter having more than one inflatable member; and
at least one atherotome structure attached to the balloon catheter and configured to extend from the balloon catheter at a plurality of diameters upon inflation of a plurality of selected inflatable members.

597. The medical device system of claim 596 where the at least one atherotome structure is fixedly attached to the balloon catheter.

598. The medical device system of claim 596 where the at least one atherotome structure is removably attached to the balloon catheter.

599. The medical device system of claim 596 where at least one of the inflatable members comprises elastomeric material.

600. The medical device system of claim 596 where at least of the inflatable members comprises non-elastomeric material.

601. The medical device system of claim 596 where at least two of the inflatable members are mounted radially adjacent each other.

602. The medical device system of claim 596 where more than two of the inflatable members are mounted radially adjacent each other.

603. The medical device system of claim 596 where at least two of the inflatable members are mounted longitudinally adjacent each other.

604. The medical device system of claim 603 where at least two of the inflatable members are mounted radially adjacent each other.

605. The medical device system of claim 596 where each of the inflatable members is independently inflatable.

606. The medical device system of claim 596 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

607. The medical device system of claim 606 further comprising at least one stenting structure and the at least one stenting structure is in contact with at the at least one core guide inflatable member.

608. The medical device system of claim 606 further comprising at least one stenting structure and the at least one stenting structure is in contact with the balloon catheter.

609. The medical device system of claim 596 further comprising a guide catheter.

610. The medical device system of claim 596 wherein each of the at least one inflatable members is independently inflatable by a separate inflation fluid tubing member.

611. The medical device system of claim 596 wherein each of the at least one inflatable members is independently inflatable by a separate inflation fluid passageway.

612. The medical device system of claim 596 wherein each of the at least one inflatable members is inflatable from a common inflation fluid passageway.

613. The medical device system of claim 596 wherein the balloon catheter is adapted to use a rapid exchange system.

614. The medical device system of claim 596 wherein the balloon catheter has a catheter wall adapted to pass a rapid exchange wire

615. The medical device system of claim 596 wherein the balloon catheter has a catheter wall adapted to allow passage of a rapid exchange wire through the wall proximally of the more than one inflatable member.

616. The medical device system of claim 615 further comprising one or more rapid exchange wires.

617. The medical device system of claim 596 further comprising a position control member configured to allow a user to direct the position of the atherotome structure from exterior of the body.

618. The medical device system of claim 596 further comprising a sleeve mounted to cover at least an atherotome structure and wherein the sleeve is configured to be substantially fixed with respect to the artherotome after insertion of the balloon catheter into the body.

619. The medical device system of claim 596 further comprising at least one constraining member longitudinally slidable along the balloon catheter, having a distal end, and wherein the constraining member is configured to be remotely slidable by a user after the balloon catheter and constraining member are introduced in the body, to slide upon the inflatable member, to constrain inflation of the inflatable member proximally of the constraining member distal end, and to permit inflation of the inflatable member distally of the constraining member distal end.

620. The medical device system of claim 619 wherein the constraining member is adapted to slide beneath the at least one atherotome structure.

621. The medical device system of claim 619 wherein the constraining member is adapted to slide above the at least one atherotome structure.

622. The medical device system of claim 619 wherein the balloon catheter has a catheter wall adapted to allow passage of a rapid exchange wire through the catheter wall proximally of the more than one inflatable member and the constraining member also comprises a wall adapted to pass a rapid exchange wire.

623. The medical device system of claim 622 comprising exactly one guidewire and that guidewire is passable through the catheter wall and the constraining member wall.

624. A method for aiding in the contraction of at least one expandable member comprising the steps of:
providing at least one expandable member for contraction, and
moving onto the at least one expandable member, a movable sleeve configured to contract the at least one expandable member to a smaller diameter.

625. The method of claim 624 wherein the at least one expandable member comprises at least one inflatable member.

626. The method of claim 625 wherein the at least one inflatable member comprise at least one inflatable balloon inflatable with an inflation fluid.

627. The method of claim 626 wherein the at least one inflatable balloon comprises at least two radially adjacent, inflatable balloons.

628. The method of claim 626 wherein the at least one inflatable balloon comprises at least two longitudinally adjacent, inflatable balloons.

629. The method of claim 627 wherein the at least one inflatable balloon further comprises at least two longitudinally adjacent, inflatable balloons.

630. The method of claim 626 wherein the movable sleeve is configured to provide an elevated pressure upon inflation fluid contained within the at least one inflatable balloon and to assist in causing said inflation fluid to flow out of the at least one inflatable balloon.

631. The method of claim 630 wherein the movable sleeve is configured to move distally upon the at least one inflatable balloon.

632. The method of claim 625 wherein the at least one inflatable member further comprises at least one elastic deflation aid configured to assist in causing said inflation fluid to flow out of the at least one inflatable balloon.

633. A kit comprising:
a.) a first balloon catheter having more than one inflatable member; and at least one stenting structure in contact with at least one of the inflatable members, wherein at least one inflatable member is configured to expand at least one stenting structure, and
b.) a second balloon catheter having more than one inflatable member.

634. The kit of claim 633 further comprising a plurality of stenting structures.

635. The kit of claim 634 wherein the plurality of stenting structures are in contact with at least one of the inflatable members of the first balloon catheter.

636. The kit of claim 633 further comprising at least one constraining member longitudinally slidable along the first balloon catheter, having a distal end, and wherein the constraining member is configured to be remotely slidable by a user after the first balloon catheter and constraining member are introduced in the body, to slide upon the inflatable member, to constrain inflation of the inflatable member proximally of the constraining member distal end, and to permit inflation of the inflatable member distally of the constraining member distal end.

637. The kit of claim 636 wherein the longitudinal movement of the constraining member adjusts the length of the inflatable member available for inflation.

638. The kit of claim 633 where at least two of the first balloon catheter inflatable members are mounted radially adjacent each other.

639. The kit of claim 633 where at least two of the first balloon catheter inflatable members are mounted longitudinally adjacent each other.

640. The kit of claim 639 where at least two of the first balloon catheter inflatable members are mounted radially adjacent each other.

641. The kit of claim 633 further comprising at least one constraining member longitudinally slidable along the second balloon catheter, having a distal end, and wherein the constraining member is configured to be remotely slidable by a user after the second balloon catheter and constraining member are introduced in the body, to slide upon the inflatable member, to constrain inflation of the inflatable member proximally of the constraining member distal end, and to permit inflation of the inflatable member distally of the constraining member distal end.

642. The kit of claim 641 wherein the longitudinal movement of the constraining member adjusts the length of the inflatable member available for inflation.

643. The kit of claim 633 where at least two of the second balloon catheter inflatable members are mounted radially adjacent each other.

644. The kit of claim 633 where at least two of the second balloon catheter inflatable members are mounted longitudinally adjacent each other.

645. The kit of claim 644 where at least two of the second balloon catheter inflatable members are mounted radially adjacent each other.

646. The kit of claim 633 further comprising a core guide member having at least one inflation area, a proximal end, a distal end, a core passageway that is fluidly connected with the at least one inflation area, and is closable distally of the at least one inflation area; having at least one core guide inflatable member with a length, the core guide inflatable member surrounding at least a portion of one of the at least one inflation area, and that is sealingly connected to the core guide member to form an inflatable region in fluid connection with the core passageway.

647. The kit of claim 646 further comprising at least one stenting structure and the at least one stenting structure is in contact with at the at least one core guide inflatable member.

648. The kit of claim 633 further comprising an elastic sleeve radially adjacent to at least one of the first balloon catheter inflatable members or the second balloon catheter inflatable members, configured to restore the at least one inflatable member to a lower profile upon deflation of the inflatable member.

649. The kit of claim 633 further comprising an elastic sleeve radially adjacent to at least one of the first balloon catheter inflatable members or the second balloon catheter inflatable members, configured to push inflation fluid out of said inflatable members.

650. The kit of claim 633 further comprising a guide catheter.

651. The kit of claim 633 further comprising at least one guide wire.

652. The kit of claim 633 wherein the balloon catheter is adapted for a rapid exchange system.

653. The kit of claim 633 wherein at least one of the first balloon catheter and the second balloon catheter is configured to pass a rapid exchange wire.

654. The kit of claim 633 further comprising kit packaging.

655. The kit of claim 633 further comprising sterilizable kit packaging.

656. A method for adjusting the length or diameter of an inflatable member in a medical device system comprising the steps of:
a.) providing the device of claim 509,
b.) placing the inflatable member at a selected site,
c.) inflating the inflatable member.

657. The process of claim 656 further comprising the step of deflating the inflatable member.

658. The process of claim 657 further comprising the step of moving the deflated inflatable member to another site in the human body, adjusting the size of the inflatable member by moving a constraining member to a second selected inflatable member size, and inflating the inflatable member.

659. The process of claim 656 further comprising the step of deflating the inflatable member.

660. A procedure for adjusting the length of an inflatable member in a medical device system comprising the steps of:
a.) providing the device of claim 509,
b.) placing a first inflatable member at a selected site in the human body, and
c.) inflating the first inflatable member to implant the stenting device.

661. The procedure of claim 660 further comprising the step of inflating the first inflatable member to reform the stenting device.

662. The procedure of claim 660 further comprising the steps of:
d.) deflating the first inflatable inflatable member,
e.) positioning an inflatable member at a selected portion of the implanted stent;
f.) inflating an inflatable member to reform the shape of the implanted stenting device, and
g.) deflating the inflatable member.

663. The procedure of claim 662 wherein the step of positioning an inflatable member comprises positioning the first inflatable member.

664. The procedure of claim 662 wherein the step of positioning an inflatable member comprises positioning a second inflatable member.

665. The procedure of claim 662 wherein the step of inflating an inflatable member further comprises tapering the implanted stenting device.

666. The procedure of claim 663 wherein the step of inflating the first inflatable member further comprises tapering the implanted stenting device.

667. The procedure of claim 664 wherein the step of inflating a second inflatable member further comprises tapering the implanted stenting device.

668. The procedure of claim 660 further comprising the step of deflating the inflatable member.

669. The procedure of claim 662 further comprising the steps of:
h.) placing an inflatable member at a second selected site in the human body,
i.) sliding a constraining member on the inflatable member of step h. until a selected inflatable member length is achieved,
j.) sliding a stent delivery sleeve having at least one stenting device on its exterior to the selected site; and
k.) inflating the inflatable member of step h. to implant the stenting device.

670. The procedure of claim 669 further comprising the step of deflating the inflatable member of step h.

671. The procedure of claim 660 further comprising the steps of:
d.) deflating the first inflatable member,
e.) positioning an inflatable member at a second selected site,
f.) sliding a constraining member on the inflatable member of step e.) until a selected inflatable member length is achieved,
g.) inflating the inflatable member of step e.) to reform the shape of the implanted stenting device, and
h.) deflating the inflatable member of step e.).

672. The procedure of claim 671 wherein the step of positioning an inflatable member comprises positioning the first inflatable member.

673. The procedure of claim 671 wherein the step of positioning an inflatable member comprises positioning a second inflatable member.

674. The procedure of claim 671 wherein the step of inflating an inflatable member further comprises tapering the implanted stenting device.

675. The procedure of claim 672 wherein the step of inflating the first inflatable member further comprises tapering the implanted stenting device.

676. The procedure of claim 673 wherein the step of inflating a second inflatable member further comprises tapering the implanted stenting device.

678. The stent delivery system of claim 106 wherein the controllably expandable assembly comprises a plurality of inflatable members configured to expand at least one of the at least one stents to more than one selected diameter and to more than one selected diameter without removing the expandable assembly from the human body.

679. The stent delivery system of claim 678 wherein the controllably expandable assembly comprises a plurality of inflatable balloons.

680. The balloon expansion system of claim 126 wherein the valving device is configured to inflate at least one balloon at an inflation position, and wherein a set of positions is configured to inflate a set of balloons resulting in a different effective length and diameter of inflated balloons.

681. The balloon expansion system of claim 680 further comprising at least one stent configured to be deployed by the set of balloons resulting in a different effective length and diameter of inflated balloons.

682. The balloon expansion system of claim 126 wherein the valving device is configured to inflate to inflate a set of balloons to different diameters and further comprising at least one atherotome configured to be deployed by the set of balloons of different diameters and further comprising at least one stent configured to be deployed by the set of balloons of different diameters.
